(19) United States
(12) Patent Application Publication
(10) Pub. No.: US 2025/0332370 A1
(43) Pub. Date: Oct. 30, 2025

(54) A BREATHING CIRCUIT

(71) Applicants: David John Love, Durban North (ZA); Fisher & Paykel Healthcare Limited, Auckland (NZ)

(72) Inventors: David John Love, Auckland (NZ); Brett John Huddart, Auckland (NZ); Jonathan David Harwood, Auckland (NZ); Andrew Paul Maxwell Salmon, Auckland (NZ); Peter Lawrence Grylls, Auckland (NZ); Daniel John Smith, Auckland (NZ); Mark Reeves, Auckland (NZ)

(21) Appl. No.: 18/837,129

(22) PCT Filed: Feb. 10, 2023
(86) PCT No.: PCT/NZ2023/050009

§ 371 Date: Aug. 8, 2024

Related U.S. Application Data
(60) Provisional application No. 63/309,446, filed on Feb. 11, 2022.

(51) Int. Cl. A61M 16/08; A61M 16/00; A61M 16/06; A61M 39/24

(52) U.S. Cl. CPC A61M 16/0875; A61M 16/0003; A61M 16/0616; A61M 2016/0015; A61M 39/24; A61M 2202/0007; A61M 2202/02; A61M 2205/3303; A61M 2205/3327; A61M 2205/3344; A61M 2230/005; A61M 2230/40

(57) ABSTRACT

The present disclosure relates to a breathing circuit and a method for providing respiratory support to a patient. The breathing circuit and method can be used in any type of breathing therapy including, for example unsealed respiratory therapy such as high flow therapy, and scaled respiratory therapy such as continuous positive air(way) pressure (CPAP) therapy, and bilevel positive air pressure therapy where the inspiratory and expiratory pressures differ. The breathing circuit includes first and second passageways that can convey a breathing gas to a patient interface, wherein the first passageway is connectable to a first gas source to supply a first gas, and the second passageway is connectable to the first gas source and to a second gas source.

### RELATED APPLICATION

This patent application claims priority to U.S. provisional patent application No. 63/309,446 entitled A Breathing Circuit, filed Feb. 11, 2022, the full contents of which are hereby incorporated in the present specification.

### FIELD

The present disclosure relates to a breathing circuit and a method for providing respiratory support to a patient. The breathing circuit and method can be used in any type of breathing therapy including, for example unsealed respiratory therapy such as high flow therapy, and sealed respiratory therapy such as continuous positive air (way) pressure (CPAP) therapy, and bilevel positive air pressure therapy where the inspiratory and expiratory pressures differ.

### BACKGROUND

Respiratory support provided by breathing circuits can help a patient to breath by opening up their airways and/or supplying specific breathing gases for a particular medicinal purpose

In the case of high flow therapy, the breathing gas may be supplied at a high flow rate (e.g. over 15 L/min) that meets or exceeds the peak inspiratory flow rate of the patient. The high flow rate may need to be provided across the whole breathing cycle, that is during both inhalation and exhalation phases to achieve the flushing benefits within the patient's anatomical deadspace, or deadspace within the breathing circuit such as the patient interface. High flow therapy is sometimes also referred to as nasal high flow (NHF), humidified high flow nasal cannula (HHFNC), high flow nasal oxygen (HFNO), high flow therapy (HFT), or tracheal high flow (THF).

Some traditional breathing circuits use a mixed breathing gas including a blend of air and oxygen gas that is supplied to a patient via an inspiratory tube. The required oxygen saturation levels in the patient's blood can be achieved by adjusting the ratio of the oxygen in the oxygen/air blend. However, a problem with this breathing circuit is the positive pressure and/or high flow rates experienced by the patient is the result of a supply of the mixed breathing gas during both inhalation and exhalation, which results in a significant wastage of the oxygen gas.

There is therefore a need to provide an alternative breathing circuit that can reduce the wastage of a therapeutic supplement gas.

### SUMMARY

An embodiment relates to a breathing circuit for providing respiratory support to a patient, the breathing circuit including:
first and second passageways that can convey a breathing gas to a patient interface, wherein the first passageway is connectable to a first gas source to supply a first gas, and the second passageway is connectable to the first gas source and to a second gas source to supply a second gas to the patient interface, and
a flow assembly that is operably connected to the first and second passageways to cause the first and second gases to flow along the second passageway during patient inhalation and to cause the first gas to flow along the first passageway during patient exhalation.

An embodiment relates to a breathing circuit for providing respiratory support to patient, the breathing circuit including:
first and second passageways that can convey a breathing gas to a patient interface, wherein the first passageway is connectable to a first gas source to supply a first gas, and the second passageway is connectable to the first gas source and to a second gas source to supply the first gas and a second gas respectively,
wherein the first passageway can convey the first gas during patient exhalation and the second passageway can convey the first gas and the second gas during patient inhalation.

An embodiment relates to a breathing circuit for providing respiratory support to a patient, the breathing circuit including:
first and second passageways that can convey a breathing gas to a patient interface, wherein the first passageway is connectable to a flow assembly to supply a first gas, and the second passageway is connectable to the flow assembly to supply the first gas and a second gas,
wherein the flow assembly and the first passageway are configured to supply the first gas to the patient interface during patient exhalation, and the flow assembly and the second passageway are configured to supply the first gas and the second gas to the patient interface during patient inhalation.

An embodiment relates to a breathing circuit for providing respiratory support to a patient, the breathing circuit including:
first and second passageways that can convey a breathing gas to a patient interface, and a flow assembly that is connectable to a first passageway to supply a first gas during patient exhalation, and is connectable to a second passageway to supply the first gas and a second gas during patient inhalation.

The embodiments described in paragraphs [0007], [0008], and may include any one or a combination of the features described herein.

One possible characteristic of the breathing circuit is that the first gas can be supplied to the patient interface during inhalation and exhalation and the first gas and the second gas can be supplied during inhalation. This makes the breathing circuit suitable for high flow therapy applications in which the second gas is supplied during inhalation where it can have a therapeutic benefit, such as increased blood oxygen saturation levels. This also makes the breathing circuit suitable for positive pressure therapy applications where during exhalation, any leak from the circuit such as intentional mask leak, or intentional leak through exhalation ports, including bias holes, is unlikely to comprise a high percentage of the second gas. Rather the vented gas will more likely be first gas from the first passageway.

Another characteristic of the breathing circuit is that the first and second passageway can convey the breathing gas to the patient interface at all times, and can convey the second gas to the patient interface independently of the first gas being conveyed to the patient interface by the first passageway. That is to say, supply of the first gas to the patient interface can be maintained at all times via the first and second passageways and can be independent of supply of second gas to the patient interface.

The patient interface may be any patient interface capable of venting the exhaled gases and any surplus in the breathing gas supplied to the interface.

In one example, the patient interface may be an unsealed patient interface. Examples include: nasal cannula, a tracheostomy interface/tube that are inserted into the neck of a patient, an oral mask that allows venting through the nasal passage, a nasal mask that allows venting through the mouth, an unsealed face mask and so forth. Unsealed patient interfaces are well suited for delivering high flow therapy.

In another example, the patient interface may be a sealed patient interface. Examples include: a full-face mask (also known as an oro-nasal mask), a sealed nasal cannula, a sealed oral mask, a sealed nasal mask, a nasal pillows interface, or a tracheostomy member.

Throughout this specification, the term “flow assembly” refers to at least one element that can be connected to one or both of the first and second passageways to form at least part of the breathing circuit, or similarly the element may be connected to another element of the flow assembly to form at least part of the breathing circuit. To avoid any doubt, these elements form part of the breathing circuit irrespective of whether the elements are described as being included in the flow assembly. Similarly, one or more elements that are described as being parts of the flow assembly may also be regarded as being elements of the breathing circuit, in which case the term “flow assembly” may be substituted with the term “breathing circuit” if it suits. In addition, the elements of the flow assembly or breathing circuit may be provided as an apparatus that can be connected to the first and/or second passageways to connect the apparatus to the patient interface.

In addition, throughout this specification, the first and/or second passageways may be described as: i) the first and/or second flow passageway including an element of the flow assembly or another element not being part of the flow assembly, such as a sensor or controller, ii) the first and/or second passageways being connected to an element, iii) an element being completely, or at least partially in the first and/or second passageway, or iv) an element being on the first and/or second passageway. In these instances, the element may or may not form part of the respective passageway. Example of the elements include a humidification chamber, a valve, an active valve mechanism, a first or second gas inlet, a reservoir, a vent, an exhalation port, joiners and so forth. Moreover, the first and/or second passageway may include multiple lengths, portions or sections that are connected together in series or parallel, with or without one or more of the elements being arranged therebetween.

Throughout this specification, the term “breathing circuit” refers to an apparatus that conducts a breathing gas to a patient. The breathing circuit may be unidirectional in the sense that any of the breathing gas that is not inhaled by the patient need not be returned to its source and can be vented. Exhaled gas can also be vented to atmosphere or captured. Similarly, if required breathing gas not inhaled may also be captured.

The flow assembly may include the first gas source. That is to say, the breathing circuit may include the first gas source that supplies the first gas. The first gas source comprises a flow generator that generates a flow of the first gas.

The flow assembly may include the second gas source. That is to say, the breathing circuit may include the second gas source that supplies the second gas source.

The flow assembly may include the first gas source and the second gas source. That is to say, the breathing circuit may include the first gas source and the second source.

The first gas may be provided by a first gas source.

For example, the first gas may be pressurized air. The first gas may be pressurized air enriched with oxygen.

The second gas may be provided by a second gas source. The breathing circuit may include the second gas source that supplies the second gas. The flow assembly may also include the second gas source that supplies the second gas.

For example, the second gas may be pressurized oxygen gas.

In another example, the second gas may be a pressurized gas including one or any combination of: oxygen gas, heliox, or an anaesthetic gas. The anaesthetic gas could be nitrous oxide or a 50:50 mixture of nitrous oxide and oxygen gas.

Pressurized oxygen gas may be supplied from a liquified oxygen source, a bottled oxygen source or from an oxygen concentrator source.

The second gas source may supply the second gas to the second passageway during inhalation. Similarly, the second gas source may supply the second breathing gas.

The second gas source may supply the second gas to the second passageway during patient exhalation.

The second gas source may supply the second gas to the second passageway during patient inhalation and exhalation. Similarly, the breathing circuit may supply the second gas to the second passageway during patient inhalation and exhalation.

The second gas source may supply/deliver the second gas to the second passageway at a constant rate during a complete breathing cycle of the patient. That is, at a constant rate during both patient exhalation and patient inhalation.

The second gas can be stored in the second passageway during patient exhalation whilst the first gas is being supplied to the patient interface via the first passageway. That is to say, accumulation of the second gas in the second passageway can occur independently of the supply of the breathing gas to the patient.

The second passageway may receive a volume of the second gas during patient exhalation where the second gas is stored during the patient exhalation. The second gas stored in the second passageway may be supplied to the patient interface during patient inhalation. The second flow assembly may supply the second gas to the second passageway at a variable flow rate. For example, the flow assembly may include a flow controller, such as a control valve, that can be operated to vary the rate at which the second gas is supplied to the second passageway.

In one example, the flow assembly may be configured to inhibit the first gas from flowing along the first passageway during patient inhalation. For example, the flow assembly may have a valve that is operable to inhibit flow along the first passageway during patient inhalation. That is to say, the breathing circuit may have a valve that is operable to inhibit flow along the first passageway during patient inhalation. The valve may be an actively-controlled valve. The valve could be, for example, a solenoid valve or a diaphragm valve. Examples of other suitable valves include a shuttle valve, a spool valve, a ball valve, a gate valve, a butterfly valve, a switch valve and so forth.

The flow assembly may also include an outlet, hereinafter referred to as a vent, for venting part or all of a residual breathing gas from the second passageway during patient exhalation. The residual gas can include any of the first and/or the second gas in the second passageway not inhaled or vented during patient inhalation. The vent may be located on the second passageway. The vent may be located downstream of the where the second gas enters the second passageway, so that the second gas entering the second passageway during patient exhalation displaces the residual gas though the vent, in which the breathing gas displaced through the vent includes any of the first and/or the second gas in the second passageway not inhaled or vented during patient inhalation. Suitably, the vent may be located on the proximal portion of the second passageway. More suitably, the vent may be located on the proximal portion of the second passageway and upstream of an active valve mechanism that is located in a proximal portion of the second passageway.

A purpose of the vent is to discharge the residual breathing gas from the second passageway at a rate at which the second gas enters the second passageway. Moreover, the vent may be suitable for venting the residual gas when the active valve mechanism prevents flow from the second passageway to the patient interface during patient exhalation. The vent can be included in the breathing circuit that has either a sealed or unsealed patient interface.

The outlet may be any one or a combination of a control valve, PEEP (positive end-expiratory pressure) valve, an aperture of fixed size, or a controlled outlet. An example of the controlled vent may be a constant flow valve that maintains a substantially constant venting flow across a range of pressures.

The flow assembly may also include an exhalation port for venting exhaled gas from the flow assembly. For instance, when the breathing circuit has a sealed patient interface. The exhalation port may also vent first gas from the first passageway to prevent overpressurising the patient interface.

The exhalation port may be located on the patient interface, suitably the patient interface. For example, bias holes in the patient interface, or a dedicated port on the interface. This arrangement has the possible characteristic of minimizing dead space and therefore rebreathing of the exhaled gas.

The exhalation port may be located on the first passageway, such as on a proximal portion of the first passageway. This arrangement has the characteristic of minimizing dead space and reducing the likelihood of the second gas being vented without being inhaled by the patient.

The exhalation port may be located on the first passageway, such as on a distal portion of the first passageway. This arrangement has the characteristic of further reducing the likelihood of the second gas inadvertently being leaked from the circuit as the second gas would need to be conveyed from the second passageway and along the first passageway to the distal portion of the first passageway.

The second passageway may include a non-return valve to inhibit the residual breathing gas from flowing upstream, that is in a direction opposite to the direction of flow of the first gas. In one example, the non-return valve may be positioned at a distal portion of the second passageway and upstream of the second gas inlet. In another example, the non-return valve may be positioned at a proximal portion of the second passageway and downstream of the second gas inlet.

The flow assembly may be operable to adjust the flow of the first gas in the first and the second passageways based in the breathing cycle of the patient. That is, based on patient inhalation and patient exhalation.

The flow assembly may be operable to adjust a parameter of the first gas in the first passageway. For example, the flow generator may be adjustable to adjust the flow rate of the first gas supplied to either one or both of the first passageway and the second passageway. For instance, during high flow therapy using an unsealed patient interface. In another example, the flow generator may be operable to adjust the pressure of the first gas supplied to the first passageway. For instanced during CPAP therapy or bi-level pressure therapy using a sealed patient interface.

The flow assembly may be operable to adjust a parameter of the first gas in the second passageway. For example, the flow generator may be operable to adjust the flow rate of the first gas supplied to either one or both of the first passageway and the second passageway. For instance, during high flow therapy using an unsealed patient interface. In another example, the flow generator may be operable to adjust the pressure of the first gas supplied to the second passageway. For instanced during CPAP therapy or bi-level pressure therapy using a sealed patient interface.

The flow generator may be connectable to the first and second passageways to divide flow from the flow generator to the first and the second passageways. For example, a split joiner such as a T-joiner or Y-joiner may divide the flow from the outlet of the flow generator to the first and the second passageways.

In one situation, the flow assembly may be operable to increase flow of the first gas in the first passageway during patient inhalation, and decrease flow of the first gas in the first passageway during patient exhalation.

In one example, there may be no or little flow of the first gas in the first passageway during patient inhalation and more flow during patient exhalation. The flow of the first gas in the first passageway during patient exhalation may be high flow.

Similarly, there may be no or little flow of the first gas in the second passageway during exhalation and more flow during patient inhalation. In one example, the flow of the first gas in the second passageway during patient inhalation may be a controlled high flow. High flow therapy may be provided using an unsealed patient interface. In another example, the flow of the first gas in the second passageway during patient inhalation may be a controlled pressurized flow. For instance, CPAP therapy or bi-level pressure therapy may be provided using a sealed patient interface.

The flow assembly may include one or more valves to adjust the flow of the first gas in the first passageway and the second passageway.

The flow assembly may be operable to alternate flow of the first gas from the flow generator between the first passageway and the second passageway.

For example, the flow assembly may include one or more valves to alternative the flow of the first gas between the first passageway and the second passageway.

For example, the one or more valves may include two valves in which one valve is located in the first passageway, and a second is located on the second passageway.

For example, the one or more valves may include a three-way valve having one inlet connectable to the first gas source and two outlets, in which one of the outlets is connectable to the first passageway and the second outlet is connectable to the second passageway.

The one or more valves may be an active valve mechanism.

Alternating flow between the first and the second passageways may include, for example:
i) there is substantially no, or little flow, of the first gas in the first passageway during inhalation, and there is flow of the first gas in the second passageway during inhalation, and
ii) there is substantially no or little flow of the first gas in the second passageway during exhalation, and there is flow of the first gas in the first passageway during exhalation.

Throughout this specification the terms “no or little flow” may refer to there being insignificant flow for respiration of the patient, or compared to respiration of the patient.

In one example,
i) there is no flow of the first gas in the first passageway during inhalation, and there is flow of the first gas in the second passageway during inhalation, and
ii) there is no flow of the first gas in the second passageway during exhalation, and there is flow of the first gas in the first passageway during exhalation.

The flow assembly may include an active valve mechanism to adjust the flow of the first gas in the first passageway and the second passageway.

The flow assembly may include an active valve mechanism to alternate flow of the first gas between the first passageway and the second passageway. One of the characteristics of this feature is that the first gas can be supplied to the patient interface via the first or the second passageways and independently of whether the active valve mechanism has the first passageway or the second passageway opening for flow of the breathing gas to the patient interface.

The active valve mechanism may include an actively-controlled valve. The actively-controlled valve may be, for example, include, a solenoid valve, a diaphragm valve, a shuttle valve, a spool valve, a ball valve, a gate valve, a butterfly valve, a switch valve, a three-way valve, and so forth. The actively-controlled valve may be adjusted by any suitable actuator, such as a solenoid actuator, a pneumatic actuator, an electric motor and so forth. The pneumatic actuator may, in one example, be operated by a dedicated source of pressurized gas. In another example, the pneumatic actuator may be operated by gas flow from the flow generator. In yet another example, the pneumatic actuator may be operated by a second gas supplied by the second gas source. In any event, a separate supply line connects the respective gas source to the pneumatic actuator for operation thereof. This avoids breathing gas in the first and/or second passageways not being used to operate the first and second actuators.

The active valve mechanism may be located in distal portions of the first and second passageways. That is to say, the first and second passageways have proximal and distal portions relative to the patient interface, and the active valve mechanism may be located in the distal portions of the first and second passageways.

The active valve mechanism may be located in proximal portions of the first and second passageways.

The flow assembly may have a second gas inlet in the second passageway downstream of the active valve assembly.

The flow assembly may have a second gas inlet in the second passageway upstream of the active valve assembly.

In one example, the second gas inlet may be located in a distal portion of second passageway. In this situation, the second gas can flow toward the patient interface during patient exhalation and be stored therein. There may be no second gas or only a minor portion of the second gas stored in the second passageway after patient inhalation that form part of the residual gas that can be vented from the second passageway during patient exhalation. That is, there is no or little venting of the second gas from the patient interface or from the first passageway during patient exhalation. The first gas downstream of a head of the second gas may be vented from the second passageway during patient exhalation.

In another example, the second gas inlet may be located in a proximal portion of the second passageway. In this situation, the second gas can be conveyed in the second passageway in a direction away from the patient interface during patient exhalation and be stored in the second passageway. There may be no second gas vented from the second passageway during patient exhalation. However, there may be some of the first gas vented from the second passageway during patient exhalation.

In one example, the second gas inlet may be located on an element that forms part of the second passageway.

In one example, wherein the second gas inlet is on a reservoir that is in the second passageway.

In another example, the second gas inlet supplies the second gas into the reservoir that is in the second passageway.

The active valve mechanism may include a three-way valve to alternate flow of the first gas between the first passageway and the second passageway.

For example, the three-way valve may have a first port that is connectable to the first passageway, a second port that is connectable to the second passageway, and a third port that is connectable to the patient interface.

The three-way valve may be connected to a proximal portion of the first and second passageways.

The three-way valve may be operable so that only one of the first port and the second port is openable at the same time, thereby allowing the flow from the first passageway and the second passageway to alternate. For instance, the first port is opened, the second port is closed, i.e., during patient exhalation, and vice versa, the first port is closed, the second port is opened, i.e., during patient inhalation. In either situation, the third port may be opened.

The flow assembly may be configured so that when the second port of the three-way valve is closed, i.e. during patient exhalation, the vent of the second passageway is operable.

In another example, the three-way valve may have a first port that is connectable to the first passageway, a second port that is connectable to the second passageway, and third port that is connectable to a first gas source.

The three-way valve may be connected to a distal portion of the first and second passageways.

The three-way valve may be operable so that only one of the first port and the second port is openable at the same time, thereby allowing the flow from the first passageway and the second passageway to alternate. For instance, the first port is opened, the second port is closed, i.e. during patient exhalation, and vice versa, the first port is closed, the second port is opened, i.e. during patient inhalation. In either situation, the third port may be opened.

In yet another example, the active valve mechanism may include first and second control valves in the first and second passageways to control flow of the first gas to the passageways.

The first and second control valves may be opened and closed alternately. That is, the first control valve opened and the second control valve closed during patient exhalation, and vice versa, the first control valve closed and the second control valve opened during patient inhalation.

In this example, the flow assembly may include a flow splitter that splits flow from the first gas source between the first and second passageways. That is to say, the flow generator has an output that is split between the first passageway and the second passageways.

In one example, the flow generator includes a first flow generator that conveys the first gas along the first passageway, and a second flow generator that conveys the first gas along the second passageway.

The flow assembly may include a third passageway interconnecting the patient interface and a joiner that merges the first and the second passageways. The joiner may be one or more tube connecters.

The flow assembly may include a flow generator that provides flow of the first gas to the first and second passageways. In one example, at least one of the first and the second passageways is opened to allow the first gas to be supplied to the patient interface at any time. In this case, the flow generator can supply respiratory therapy to the patient irrespective of whether the first or the second passageway is opened to the patient interface.

The flow generator may include a flow regulator for controlling the flow of the first gas.

In one example, the flow generator may have a single blower.

In one example, the flow generator has a single flow output that is split between the first and the second passageways.

In another example, the flow generator has an output that is split between the first passageway and the second passageways.

In another example, the flow generator may have two flow outputs, and each flow output can be supplied to either one of the respective first and second passageways. In one example, each flow output may be driven by a separate blower, that is the flow generator may have two blowers. The two blowers may be separate devices, for example two Nasal High Flow devices. The blowers may be controlled by a single controller, or each controlled separately. Alternatively, the two blowers may be integrated into a single device with a single controller.

In another example, each flow output may be provided by a single blower. Each blower may be controlled separately or together.

In another example, the flow generator may include a first flow generator that conveys the first gas along the first passageway, and a second flow generator that conveys the first gas along the second passageway.

The breathing circuit may include a sensor for detecting the breathing cycle of the patient. That is, when patient inhalation and patient exhalation is occurring.

The sensor may have an output signal that is used to operate the active valve mechanism. That is, the output signal of the sensor can be used to adjust the flow of the first gas in the first passageway and the second passageway, and suitably to alternate the flow of the first gas between the first passageway and the second passageway.

The sensor may have an output signal that is used to operate the flow generator.

In one example, the output signal can be used to adjust the flow rate of the first gas supplied by the flow generator. For example, during high flow therapy using an unsealed patient interface.

In another example, the output signal can be used to adjust the pressure of the first gas supplied by the flow generator. For example, during CPAP or bi-level pressure therapy using an sealed patient interface.

The output signal may be used to adjust the pressure of the first gas supplied by the flow generator between a first pressure being an IPAP (inspiratory positive airway pressure) and a second pressure being an EPAP (expiratory positive airway pressure).

In one example, the sensor may comprise at least one gas sensor that can detect a gas property of the breathing gas supplied to the patient interface, a property of exhaled gases, or a property of gases being vented from the breathing circuit. Examples of the gas property include: gas flow rate, gas pressure, gas temperature, gas humidity or gas concentration, such as oxygen or carbon dioxide concentration. The sensor may include a pressure sensor, suitably at or near an outlet of the flow generator. For example, the pressure sensor may be downstream of a blower within the flow generator. In another example, the pressure sensor may be located on the patient interface. The sensor may include a flow sensor, suitably at or near an outlet of the flow generator. For example, the flow sensor may be upstream or downstream of the blower within the flow generator. The output signal can then be used to determine if the patient is inhaling or exhaling and, in turn, the first gas can be supplied to the first passageway during exhalation and the first gas supplied to the second passageway during inhalation at a desired flow rate or a desired pressure. The flow generator can supply the first gas at a suitable pressure when the patient interface is a sealed patient interface. The flow generator can supply the first gas at a suitable flow rate when the patient interface is an unsealed patient interface.

In one example, wherein the sensor may include at least one sensor, and suitably, multiple sensors located at a selection of the following locations: i) at or near the patient interface; ii) at or near an outlet of the flow generator, iii) in the first passageway, and iv) in the second passageway.

In one example, the sensor may include a pressure sensor located at or near an outlet of the flow generator, or located on the patient interface.

In an example, the sensor may include a pressure sensor at the second gas inlet, or at a vent in the first passageway, or at an exhalation portion.

In an example, the sensor may include a flow sensor located at one or more of the following: i) at or near an outlet of the flow generator, ii) at or near the patient interface; iii) at the second gas inlet.

The gas sensors may be located at, or proximal to, the first gas source, such as a blower. Additionally or alternatively, a flow sensor, a pressure sensor, or a temperature sensor may also be located at other locations of the breathing circuit, such as at or near the patient interface.

In another example, the sensor may be an external sensor that detects a respiratory parameter independently of a gas property. For example, the external sensors that can be attached to the abdomen of a patient to indicate if the patient is inhaling and/or exhaling include pulse oximeter sensors, accelerometers sensors, piezoelectric sensors, capnography sensors, bioimpedance sensors or so forth. Similarly, output signals from the external sensors can be used to determine if the patient is inhaling or exhaling and, in turn, the first gas supplied to the first passageway during exhalation and the first gas supplied the second passageway during inhalation.

The breathing circuit may include a controller that receives the output signal of the sensor(s), and the controller has a processor that calculates the period of inhalation and/or exhalation, and in turn, produce a control output that is used to operate the active valve mechanism.

The controller may calculate the period of inhalation, and generate an output signal to operate the active valve mechanism that minimizes the delay between the active valve mechanism supplying the first gas to the second passageway and the patient receiving the breathing gas from the second passageway at the start of patient inhalation.

In one example, the flow generator may be adjusted to control the flow rate of the first gas based on the output signal of the controller. For instance, during high flow therapy using an unsealed patient interface.

In another example, the flow generator may be adjusted to control the pressure of the first gas based on the output signal of the controller. For instance, during CPAP or bi-level therapy using a sealed patient interface.

In the situation where the breathing circuit includes the flow generator that supplies the first gas, the flow generator may be adjusted to adjust the flow rate of the first gas based on the output signals of the sensor(s).

The flow assembly may include a humidification device for humidifying part of, or all of, the breathing gas.

The humidification device may be configured to humidify the first gas in the first passageway.

The humidification device may be configured to humidify only the first gas in the second passageway. In this situation, the second gas is supplied to the second passageway downstream of the humidification device.

The humidification device may be configured to humidify the first and second gases in the second passageway. In this situation, the humidification device is located downstream of the second gas entering the second passageway.

In the situation where the humidification device humidifies both the first and second gases, the humidification device may have dedicated dual chambers, one for each passageway. In this instance, the chambers form part of the passageways.

In one example, the second gas inlet is on the humidification device that is in the second passageway.

In one example, the second gas inlet supplies the second gas into the humidification device that is in the second passageway.

In the situation where the flow assembly has a third passageway, the humidification device may humidify the breathing gas in the third passageway.

In the situation where the flow assembly has a splitter that splits flow of the first gas between the first and the second passageways, the humidification device may be located upstream of the splitter. In one example, the humidification device may have a single chamber for humidifying the first gas prior to the splitter. In another example, the humidification device may have dual chambers, namely a first chamber of the humidifying the first gas supplied to the first passageway, and a second chamber of the humidifying the second gas supplied to the second passageway. A characteristic of the dual chamber humidification device is that first gas supplied to the respective first and second passageways can be humidified to different extents.

The second passageway may have an internal volume that is sized to deliver a desired amount. The desired amount may be a therapeutic amount of the second gas to the patient. For example, the internal volume may be approximately equal to the tidal volume of the patient. In other words, the therapeutic amount may be an amount that can be inhaled by the patient in a single breath.

For adult patients, the second passageway may have a length ranging from about 0.5 m to 2.5 m, or about a length ranging from 0.75 to 2.0 m, or a length ranging from about 1.5 to 1.8 m. The gas passageway may include a main passage of constant diameter, in which the diameter may range from about 8 to 15 mm, or the diameter is about 10 mm.

The situation where the second passageway includes a humidification device, the volume of the humidification device will also contribute to the internal volume of the second passageway.

High flow therapy, as used herein, is intended to be given its typical ordinary meaning, which generally refers to a breathing circuit supplying a targeted flow of breathing gas via an unsealed patient interface with flow rates generally intended to meet or exceed inspiratory flow of a user. The breathing gas may or may not be humidified, but is suitably humidified to increase patient comfort. Typical inspiratory flow rates for adults often range from, but are not limited to, about 15 L/min to about 60 L/min or greater. Typical flow rates for pediatric users (such as neonates, infants and children) often range from, but are not limited to, about 1 L/min per kilogram of user weight to about 3 L/min per kilogram of user weight or greater. High flow therapy can also optionally include gas mixture compositions including supplemental oxygen and/or administration of therapeutic medicaments which may be supplied by the second gas, alternatively by the first gas, or alternatively by the first gas and the second gas.

High flow therapy is often referred to as nasal high flow (NHF), humidified high flow nasal cannula (HHFNC), high flow nasal oxygen (HFNO), high flow therapy (HFT), or tracheal high flow (THF), among other common names. For example, in some configurations, for an adult patient ‘high flow therapy’ may refer to the supply of gases to a patient at a flow rate of greater than or equal to about 10 L/min, such as between about 10 L/min and about 100 L/min, or between about 15 L/min and about 95 L/min, or between about 20 L/min and about 90 L/min, or between about 25 L/min and about 85 L/min, or between about 30 L/min and about 80 L/min, or between about 35 L/min and about 75 L/min, or between about 40 L/min and about 70 L/min, or between about 45 L/min and about 65 L/min, or between about 50 L/min and about 60 L/min.

In some configurations, for a neonatal, infant, or child patient ‘high flow therapy’ may refer to the supply of breathing gases to a patient at a flow rate of greater than 1 L/min, such as between about 1 L/min and about 25 L/min, or between about 2 L/min and about 25 L/min, or between about 2 L/min and about 5 L/min, or between about 5 L/min and about 25 L/min, or between about 5 L/min and about 10 L/min, or between about 10 L/min and about 25 L/min, or between about 10 L/min and about 20 L/min, or between about 10 L/min and 15 L/min, or between about 20 L/min and 25 L/min. A high flow therapy apparatus with an adult patient, a neonatal, infant, or child patient, may supply gases to the patient at a flow rate of between about 1 L/min and about 100 L/min, or at a flow rate in any of the sub-ranges outlined above.

High flow therapy can be effective in meeting or exceeding the patient's inspiratory demand, increasing oxygenation of the patient and/or reducing the work of breathing. Additionally, high flow therapy may generate a flushing effect in the nasopharynx such that the anatomical dead space of the upper airways is flushed by the high incoming gases flow. The flushing effect can create a reservoir of fresh gas available for each and every breath, while minimizing re-breathing of carbon dioxide, nitrogen, etc. High flow therapy can also increase expiratory time of the patient due to pressure during expiration. This in turn reduces the respiratory rate of the patient.

The pressure of the breathing gas, and therefore the first gas and second gas supplied to the patient interface for CPAP therapy is often greater than 2 cmH₂O, such as between about 2 and about 40 cmH₂O, and suitably between about 4 and about 30 cmH₂O.

Elements of the breathing circuit may be preconnected or pre-assembled into a module to reduce the complexity of the circuit. The module may be connected to other elements to complete the breathing circuit, or two or more modules may be connected together, which in turn may form the breathing circuit or be connected to other elements. Elements that may be included in one or more modules include the first and second passageways, joiners of connecting the first and the second passageways, the flow generator, the sensor(s), the active valve mechanism, the humidifier(s), the second gas inlet, the patient interface, the outlet on the second passageway, the exhalation portion, and optionally the non-return valve on the first and/or second passageway. Examples of possible modules may include any one or a combination of the following:
Module 1: The first and second passageways may be preconnected at either one or both of the proximal or distal portions thereof. For instance, the first and second passageways may be preconnected by way of Y- or T-shaped joiners at either the proximal and/or distal ends of the first and second passageways.
Module 2: The first and second passageways may be preconnected with an active valve mechanism for controlling the flow of the first gas. Optionally, the proximal and/or distal ends of the first and the second passageways may be connected by T- or Y-shaped joiners in accordance with Module 1.
Module 3: The second passageway may be preconnected to a second gas inlet. Optionally, the second passageway may be preconnected in the manner described in Modules 1 or 2.
Module 4: The second passageway may be preconnected to the outlet for venting residual breathing gas. Optionally, the second passageway may be preconnected in the manner described in any one of Modules 1 to 3.
Module 5: Proximal and/or distal portions of the first passageways may have an exhalation portion. Optionally, the first passageway may be preconnected in the way described in any one of Modules 1 to 4.
Module 6: One or more sensors may be provided either one or both of the first and the second passageway. Optionally, Module 6 may also include any one of the preconnections of Modules 1 to 5.
Module 7: The patient interface may be preconnected to a joiner, such as a Y- or T-shaped joiner for connection to the first and the second passageways. Optionally, Module 5 may connected to any of Modules 1 to 6.
Module 8: Either one or both of the first and second passageways may have a non-return valve.

A method of providing respiratory support to a patient, the method including:
supplying a first gas to a first passageway connected to a patient interface during patient exhalation;
supplying a second gas to a second passageway connected to the patient interface; and
wherein supplying the first gas to the second passageway displaces at least some of the second gas in the second passageway to the patient interface during patient inhalation.

The method may further include storing the second gas in the second passageway during patient exhalation.

The method may include maintaining supply of the breathing gas in at least one of the first passageway and the second passageway at all times, so that the supplying the second gas to the patient interface via the second passageway can be controlled independently of supply of the first gas to the patient interface.

The method may include supplying of the first gas to the patient interface by the first exhalation via the first passageway, and supplying the first gas and the second gas to the patient interface via the second passageway based on the breathing cycle of the patient.

The method may include alternating supply of the first gas between the first passageway and the second passageway during patient exhalation and patient inhalation respectively.

The method may include operating an active valve mechanism to control the supply of the first gas to the first passageway and to control the supply of the first gas to the second passageway.

The method may include operating the active valve mechanism comprises operating an actively-controlled valve comprising a three-way valve to alternate flow of the first gas between the first passageway and the second passageway respectively.

The method may include operating the active valve mechanism comprises operating actively-controlled first and second control valves in the first and second passageways to control the supply of the first gas to the first and second passageways respectively. For example, the first and second control valves are opened and closed alternately, that is, the first control valve opened and the second control valve closed during patient exhalation, and vice versa, the first control valve closed and the second control valve opened during patient inhalation

The may include sensing a respiration rate (or breathing cycle) of the patient; and operating an active valve mechanism to control flow of the first gas to the first passageway and the second passageway.

The method may include sensing at least one of a gas property of the breathing gas supplied to the patient interface, a property of exhaled gases, or a property of gases being vented from the breathing circuit, in which the gas property includes any one or a combination of: gas flow rate, gas pressure, gas temperature, gas humidity or gas concentration, such as oxygen or carbon dioxide concentration; and alternating the flow of the first gas between the first passageway and the second passageway during patient exhalation and patient inhalation respectively.

The method may include calculating the period of inhalation and/or exhalation, and generating an output signal to operate the active valve mechanism. For example, the output signal generated to operate the active valve mechanism minimizes the delay between the active valve mechanism supplying the first gas to the second passageway and the patient receiving the breathing gas from the second passageway at the start of patient inhalation. The output signal can be used to operate the active valve mechanism comprises supplying the first gas to the second passageway before inhalation by a pre-emptive period.

The method may include operating a first gas source to control the flow rate and/or pressure of the first gas supplied to the first and the second passageways.

The method may include using the output signal to operate a first gas source to control the flow rate and/or pressure of the first gas supplied to the first and the second passageways.

The method may include operating the first gas source comprises operating a single flow generator to supply the first gas.

The method may include operating the first gas source comprises operating first and second flow generators, in which the first flow generator provides a stream of the first gas to the first passageway and the second flow generator provides a stream of the first gas to the second passageway.

The method may include operating a second gas source to control the flow rate and/or pressure of the second gas into the second passageway.

In one example, the second gas may be supplied to a distal portion of second passageway, and flows toward the patient interface during patient exhalation and is stored therein during patient exhalation.

In another example, the second gas may be supplied to a proximal portion of the second passageway, and flows a direction away from the patient interface during patient exhalation and is stored therein during patient exhalation.

The method may include providing high pressure therapy by controlling the flow rate of the first gas supplied to the first and second passageways and using an unsealed patient interface.

The method may include venting part or all of a residual breathing gas from the second passageway during patient exhalation.

The method may include venting the residual breathing gas via an outlet that is located downstream of the where the second gas enters the second passageway, so that the second gas entering the second passageway during patient exhalation displaces the residual gas though the outlet, in which the breathing gas displaced through the outlet includes any of the first and/or the second gas in the second passageway not inhaled or vented during patient inhalation.

The method may include providing CPAP or bi-level therapy by controlling the pressure of the first gas supplied to the first and second passageways and using a sealed patient interface.

The method may include venting exhaled gas via an exhalation port, and, if required, venting first gas from the first passageway to prevent over-pressurising the patient interface.

The method may include providing the exhalation port on the sealed patient interface.

The method may include providing the exhalation port on a proximal portion of the first passageway, which reduces the likelihood of the second gas being vented through the exhalation port without being inhaled by the patient.

The method may include providing the exhalation port on a distal portion of the first passageway, which further reduces the likelihood of the second gas inadvertently being leaked form the circuit.

The method may include humidifying the first gas in the first passageway.

The method may include humidifying the first gas in the second passageway without humidifying the second gas in the second passageway.

The method may include humidifying the first and second gases in the second passageway.

The method may include humidifying both the first and second gases in separate humidification chambers, thereby allowing the first gas and the second gas to be humidified to different extents as required.

The method may include storing the second gas during patient exhalation whilst the first gas is being supplied to the patient interface via the first passageway, so that the second gas accumulates in the second passageway independently of the supply of the breathing gas to the patient.

The statement the second gas may include one or any combination of: oxygen gas, heliox, or an anaesthetic gas.

The method may include controlling the rate at which the second gas is supplied to the second passageway.

The method may provide high flow therapy in which the first and second gases are supplied to a (adult) patient interface at a flow rate greater than or equal to about 10 L/min, such as between about 10 L/min and about 100 L/min, or between about 15 L/min and about 95 L/min, or between about 20 L/min and about 90 L/min, or between about 25 L/min and about 85 L/min, or between about 30 L/min and about 80 L/min, or between about 35 L/min and about 75 L/min, or between about 40 L/min and about 70 L/min, or between about 45 L/min and about 65 L/min, or between about 50 L/min and about 60 L/min.

The method may include providing high flow therapy in which the first and second gases are supplied to a (neonatal, infant, or child) patient interface at a flow rate of greater than 1 L/min, such as between about 1 L/min and about 25 L/min, or between about 2 L/min and about 25 L/min, or between about 2 L/min and about 5 L/min, or between about 5 L/min and about 25 L/min, or between about 5 L/min and about 10 L/min, or between about 10 L/min and about 25 L/min, or between about 10 L/min and about 20 L/min, or between about 10 L/min and 15 L/min, or between about 20 L/min and 25 L/min.

The method may include providing CPAP therapy in which the first gas and second gases are supplied to the patient interface at a pressure greater than 2 cmH₂O, such as between about 2 and about 40 cmH₂O, and suitably between about 4 and about 30 cmH₂O.

An embodiment relates to a method of providing respiratory support to a patient, the method including the steps of:
providing a breathing circuit for providing respiratory support to a patient, the breathing circuit having first and second passageways that convey a breathing gas to a patient interface, and a flow assembly that is connectable to a first passageway and is connectable to a second passageway; and
operating the flow assembly so that first passageway supplies the first gas to the patient interface during patient exhalation, and the second passageway supplies the first gas and the second gas to the patient interface during patient inhalation.

The step of operating the flow assembly may include operating an active valve mechanism to allow or inhibit the flow of a first gas to the first and second passageways. Operating the active valve mechanism may include alternating flow of the first gas between the first passageway and the second passageway during patient exhalation and inhalation.

Operating the flow assembly may include: sensing a gas flow parameter in the breathing circuit to detect the breathing cycle of the patient; and operating the active valve mechanism to deliver flow of the first gas to the first passageway during patient exhalation and to deliver flow of the first gas to the second passageway during patient inhalation.

Operating the flow assembly may include: sensing a respiration rate of the patient; and operating the active valve mechanism to deliver flow of the first gas to the first passageway during patient exhalation, and to deliver flow of the first gas to the second passageway during patient inhalation.

When the breathing circuit has a delay in supplying the breathing gas after the first gas has been delivered to the second passageway, for example due to internal volume of one or more of the components of the flow assembly and the second passageway, operating the flow assembly may include controlling the active valve mechanism to minimise any delay in delivering the breathing gas. Specifically, the active valve mechanism may be operated to deliver the first gas to the second passageway before inhalation by a pre-emptive period. That is to say, operating the flow assembly may include controlling the active valve mechanism to minimise any delay in delivering the first gas to the second passageway and the second gas being supplied to the patient interface.

The step of operating the flow assembly may include operating a second gas source by controlling the flow rate of the second gas into the second passageway.

The step of operating the flow assembly may include operating a first gas source, including a flow generator, to deliver the first gas to the first and the second passageways.

The step of operating the flow assembly may include operating a single flow generator to deliver the first gas.

The step of operating the flow assembly may include operating first and second flow generators, in which the first flow generator delivers a stream of the first gas to the first passageway and the second flow generator delivers a stream of the first gas to the second passageway.

The method described herein may include any one or a combination of the other elements described herein, and vice versa the breathing circuit described herein may include any one or a combination of the elements of the method described herein.

For example the method may include: humidifying the breathing gas using a humidification device, controlling the flow rate of the breathing gas including controlling the flow rate of the respective first and second gases, providing a non-return valve in the first and/or second passageways, and so forth.

### BRIEF DESCRIPTION OF THE DRAWINGS

These and other features, aspects, and characteristics of the present disclosure are described with reference to the drawings of certain embodiments, which are intended to schematically illustrate certain embodiments and not to limit the disclosure.

FIGS. 1 and 2 are schematic illustrations of a breathing circuit including first and second passageways in which distal portions thereof are connected to a first gas source, and a distal portion of the second passageway is connected to a second gas source. In addition, the breathing circuit including a flow assembly that allows the first and second passageways to be connected alternately to a patient interface, with the first passageway being connected in FIG. 1 and the second passageway being connected in FIG. 2. The breathing circuit in FIG. 1 also includes at least one sensor and a controller that also form part of the breathing circuit in FIG. 2 but have been omitted.

FIGS. 3 and 4 are similar to FIGS. 1 and 2 respectively, with the addition of humidification devices.

FIGS. 5 and 6 are similar to FIGS. 1 and 2 respectively, with the addition of humidification devices, and the second gas source is connected to a proximal portion of the second passageway.

FIGS. 7 and 8 are schematic illustrations of a breathing circuit including first and second passageways both having proximal portions connected to a patient interface and each passageway having a separate humidification device. In addition, the breathing circuit includes a flow assembly in which distal portions of the first passageway and the second passageway are alternately connected to a first gas source, with the first passageway being connected in FIG. 7 and the second passageway being connected in FIG. 8. A distal portion of the second passageway is connected to a second gas source. The breathing circuit in FIG. 7 includes at least one sensor and a controller that also form part of the breathing circuit in FIG. 8 but have been omitted.

FIGS. 9 and 10 are similar to FIGS. 7 and 8 respectively, except for the two humidification devices in FIGS. 7 and 8 are replaced with a single humidification device.

FIG. 11 is a schematic illustration of a breathing circuit having first and second passageways both having proximal portions connected to a patient interface. The first and second passageways are connected to a dual chamber humidification device, and a distal portion of the second passageway is connected to a second gas source. The breathing circuit also includes an active valve mechanism for adjusting the flow of the first gas in the first and second passageways to alternate flow therein, and at least one sensor and a controller. For convenience, a flow assembly may (or may not) be described in the DETAILED DESCRIPTION and elsewhere as including the active valve mechanism, the humidification device, the sensor, the controller and other elements.

FIG. 12 is a schematic illustration of a breathing circuit including a distal portion having first and second passageways, and a proximal portion extending from the distal portion having a third passageway connected to a patient interface. The breathing circuit also includes an active valve mechanism for adjusting the flow of the first gas in the first and second passageways, such as alternate flow. The breathing circuit also includes a dual chamber humidification device, at least one sensor, and a controller. For convenience, a flow assembly may (or may not) be described in the DETAILED DESCRIPTION and elsewhere as including the active valve mechanism, humidification device, the sensor, the controller and other elements.

FIG. 13 is a schematic illustration of a breathing circuit including a distal portion having first and second passageways, and a proximal portion extending from the distal portion including a third passageway connected to a patient interface and a humidification device. The breathing circuit includes an active valve mechanism for adjusting the flow of the first gas in the first and second passageways, such as alternate flow, at least one sensor, a controller, and a second gas source that is connected to a reservoir that forms part of the second passageway. For convenience, a flow assembly may (or may not) be described in the DETAILED DESCRIPTION and elsewhere as including the active valve mechanism, the humidification device, the sensor, the controller, and other elements.

FIG. 14 is a schematic illustration of a breathing circuit including first and second passageways connected to a patient interface, in which flow generators are connected to the first and second passageways that provide flow of the first gas and humidification devices, a second gas source connected to a distal portion of the second passageway, at least one sensor and a controller for controlling the breathing circuit. For convenience, a flow assembly may (or may not) be described in the DETAILED DESCRIPTION and elsewhere as including the flow generators, and other elements.

FIGS. 15 and 16 are schematic illustrations of a breathing circuit including first and second passageways in which distal portions thereof are connected to a first gas source, and a distal portion of the second passageway is connected to a second gas source. The breathing circuit also including a flow assembly that alternately connects the first and second passageways to a sealed patient interface to allow flow of the first gas thereto, with the first passageway being connected in FIG. 15 and the second passageway being connected in FIG. 16. For convenience, the flow assembly may be described in the DETAILED DESCRIPTION and elsewhere as including at least one sensor and a controller for controlling the breathing circuit.

FIGS. 17 and 18 illustrate the breathing circuit of FIGS. 15 and 16 with an exhalation port in different locations.

FIGS. 19 and 20 are the same as FIGS. 15 and 16 respectively, with the addition of humidification devices to the first and second passageways.

FIGS. 21 and 22 illustrate the breathing circuit of FIGS. 19 and 20 with an exhalation port in different locations, and the at least one sensor and the controller have been omitted.

FIGS. 23 and 24 are the same as FIGS. 15 and 16 respectively, with the addition of humidification devices to the first and second passageways, the second gas source is connected to a proximal portion of the second passageway, and a vent has been removed from the second passageway.

FIGS. 25 and 26 illustrate the breathing circuit shown in FIGS. 23 and 24 with an exhalation port in different locations, and the at least one sensor and the controller have been omitted.

FIGS. 27 and 28 are schematic illustrations of a breathing circuit including first and second passageways both having proximal portions connected to a patient interface and each passageway having a separate humidification device. In addition, the breathing circuit includes a flow assembly in which distal portions of the first passageway and the second passageway are alternately connected to a first gas source, with the first passageway being connected in FIG. 27 and the second passageway being connected in FIG. 28. A distal portion of the second passageway is connected to a second gas source, and at least one sensor and a controller are provided for controlling the breathing circuit.

FIGS. 29 and 30 illustrate the breathing circuit shown in FIGS. 27 and 28 with an exhalation port in different locations and the at least one sensor and the controller have been omitted.

FIGS. 31 and 32 are similar to FIGS. 27 and 28 respectively, with one modification being the two humidification devices in FIGS. 27 and 28 have been replaced with a single humidification device.

FIGS. 33 and 34 illustrate the breathing circuit shown in FIGS. 31 and 32 with an exhalation port in different locations and the at least one sensor and the controller have been omitted.

FIG. 35 is a schematic illustration of a breathing circuit including first and second passageways both having proximal portions connected to a patient interface. The breathing circuit also includes a flow assembly in which the passageways are connected to a dual chamber humidification device, and a second gas source is connected to a distal portion of the circuit. The flow assembly also includes an active valve mechanism for adjusting the flow of the first gas in the first and second passageways to alternate flow therebetween to the patient, at least one sensor, and a controller for controlling the breathing circuit.

FIGS. 36 and 37 illustrate the breathing circuit shown in FIG. 35 with an exhalation port in different locations and the at least one sensor and the controller have been omitted.

FIG. 38 is a schematic illustration of a breathing circuit including first and second passageways in a distal portion and a third passageway in a proximal portion being connected to the patient interface. The breathing circuit including an active valve mechanism for adjusting the flow in first and second passageways, such as alternate flow, a dual chamber humidification device, at least one sensor and a controller for controlling the breathing circuit. A flow assembly may include the active valve mechanism.

FIGS. 39 and 40 illustrate the breathing circuit shown in FIG. 38 with an exhalation port in different locations and the at least one sensor and the controller have been omitted.

FIG. 41 is a schematic illustration of a breathing circuit including first and second passageways in a distal portion and a third passageway in a proximal portion being connected to a patient interface, and a humidification device. The breathing circuit including an active valve mechanism for adjusting flow of the first gas in the first and second passageways, such as alternate flow, a second gas source connected to the second passageway, at least one sensor and a controller for controlling the breathing circuit.

FIGS. 42 and 43 illustrate the breathing circuit shown in FIG. 41 with an exhalation port in different locations and the at least one sensor and the controller have been omitted.

FIG. 44 is a block diagram of some of the elements of the breathing circuits shown in FIGS. 1 to 43. The arrows represent relationships or associations between elements. The arrows do not necessarily represent flows between elements or data signals between elements.

FIG. 45 is a block diagram of a method for providing respiratory support to a patient. The method may include using any one of the breathing circuits described herein.

### DETAILED DESCRIPTION

An embodiment will now be described in the following text which includes reference numerals that correspond to features illustrated in the accompanying Figures. To maintain clarity of the Figures, however, not all reference numerals are included in each Figure. Although certain examples are described herein, those of skill in the art will appreciate that the disclosure extends beyond the specifically disclosed examples and/or uses and obvious modifications and equivalents thereof. Thus, it is intended that the scope of the disclosure herein disclosed should not be limited by any particular examples described herein.

As mentioned above, such as in paragraph [0017], the flow assembly 24 described herein includes various elements that forms part of the breathing circuit 20, and elements of the flow assembly 24 are equally elements of the breathing circuit 20. In addition, as mentioned in above in paragraph [0018], elements that are described as being connected to the first and second passageway 21 and 22 may or may not form part of the respective passageway 21 and 22.

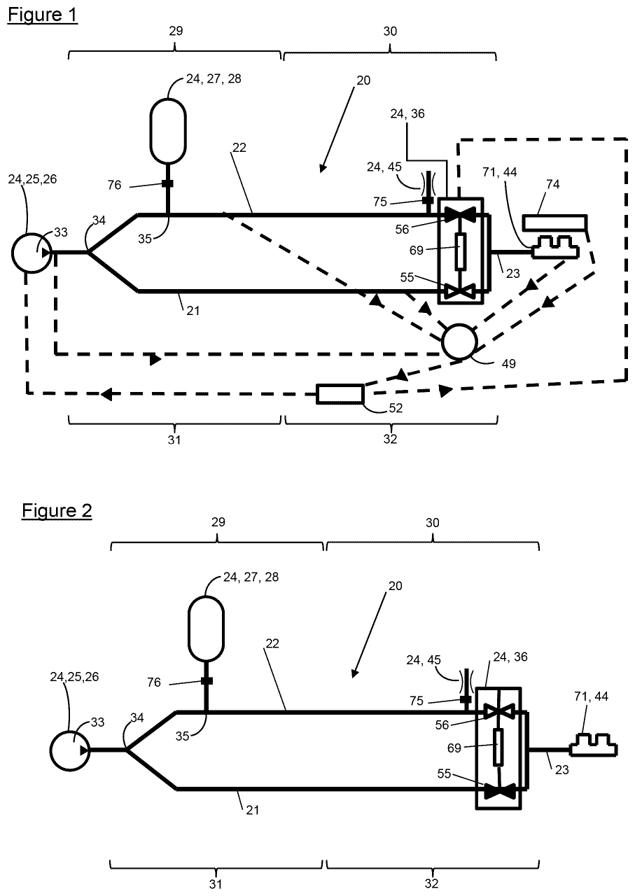

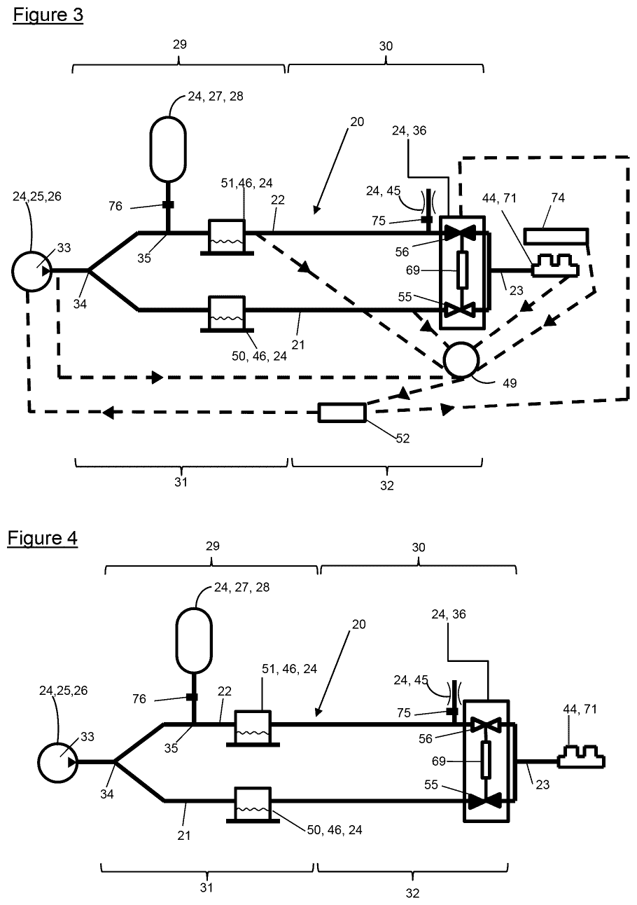

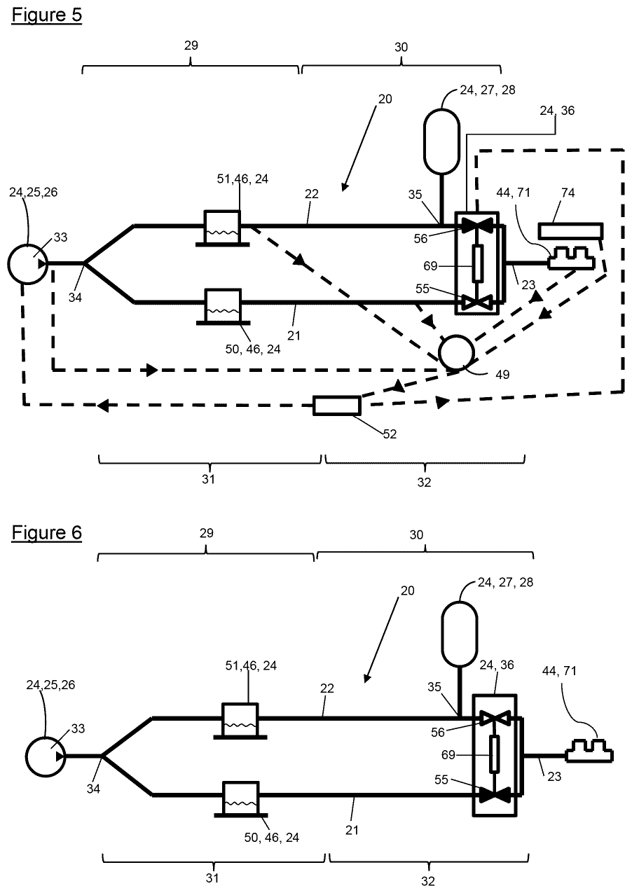

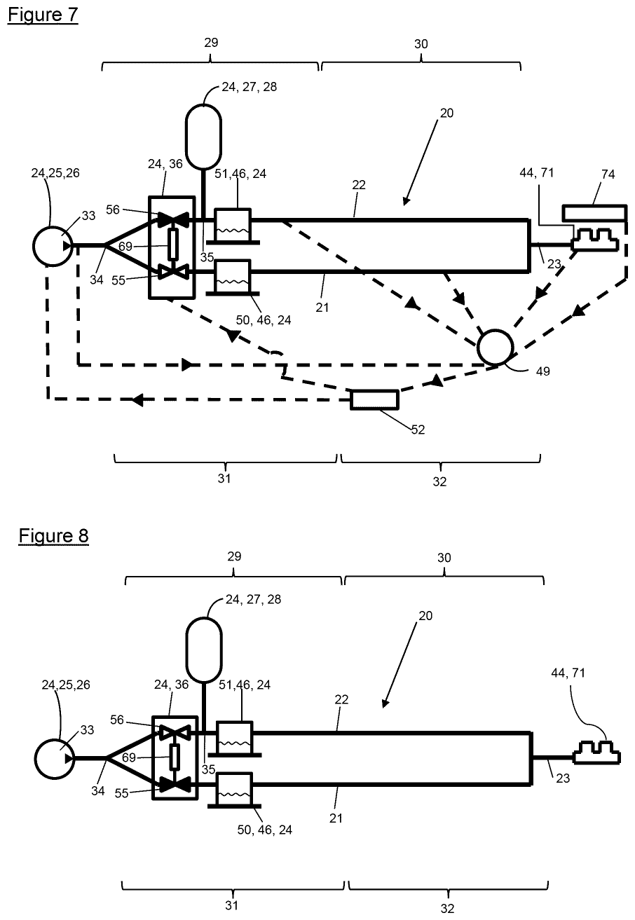

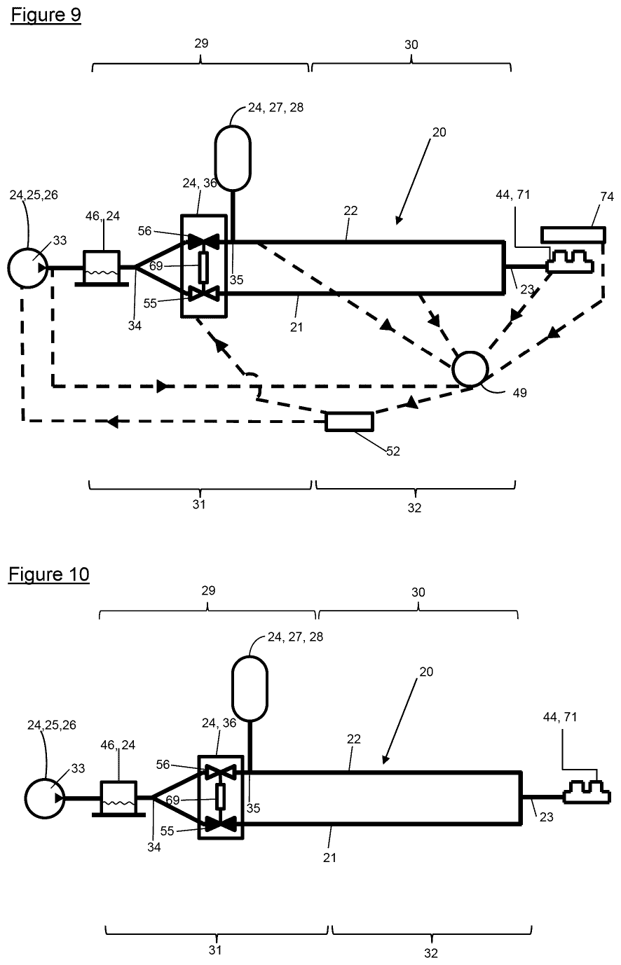

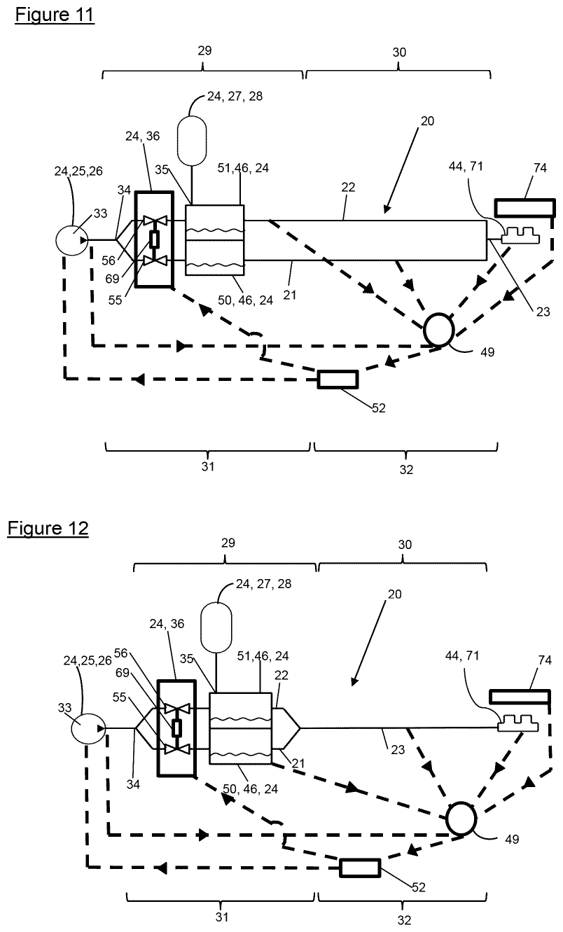

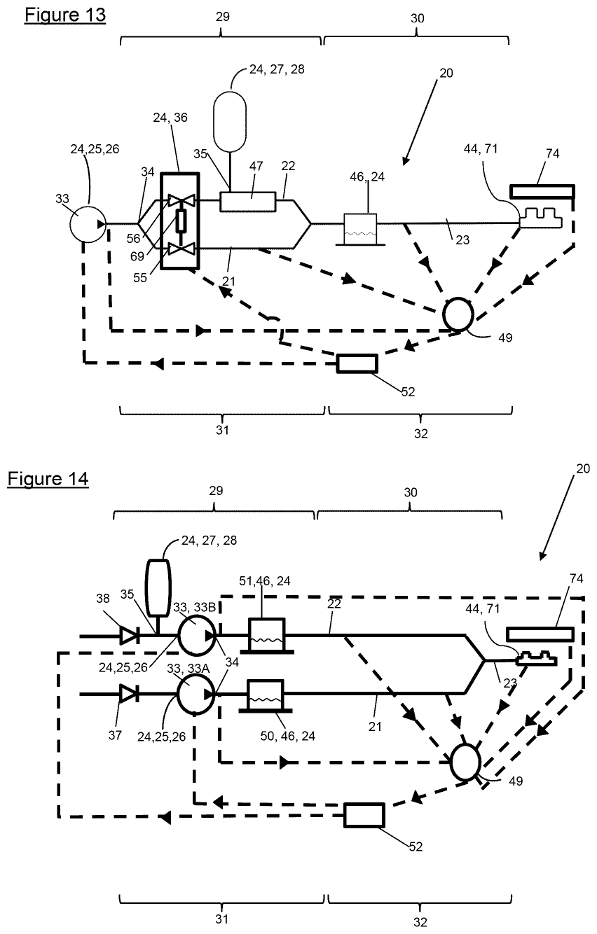

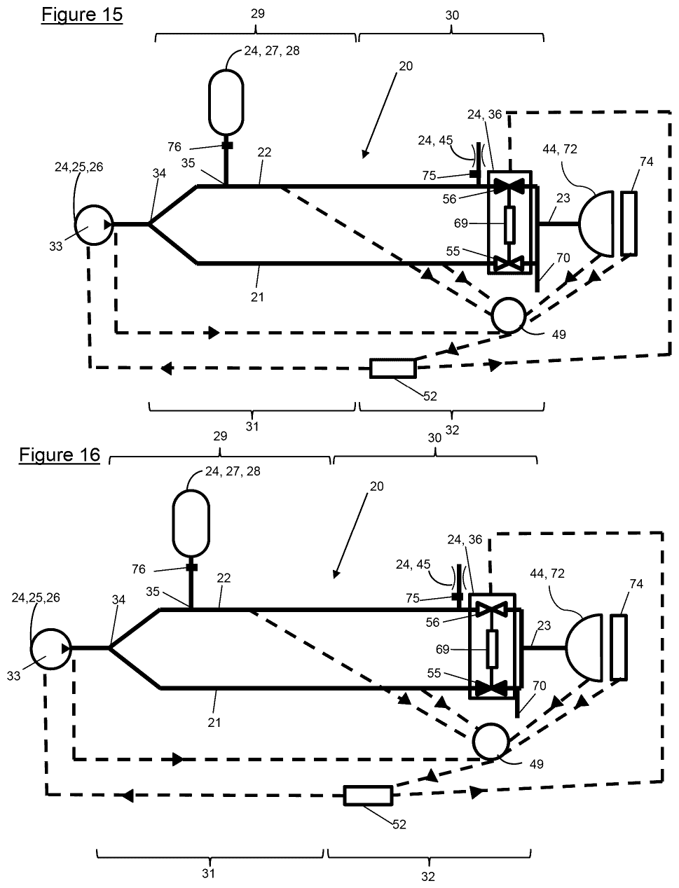

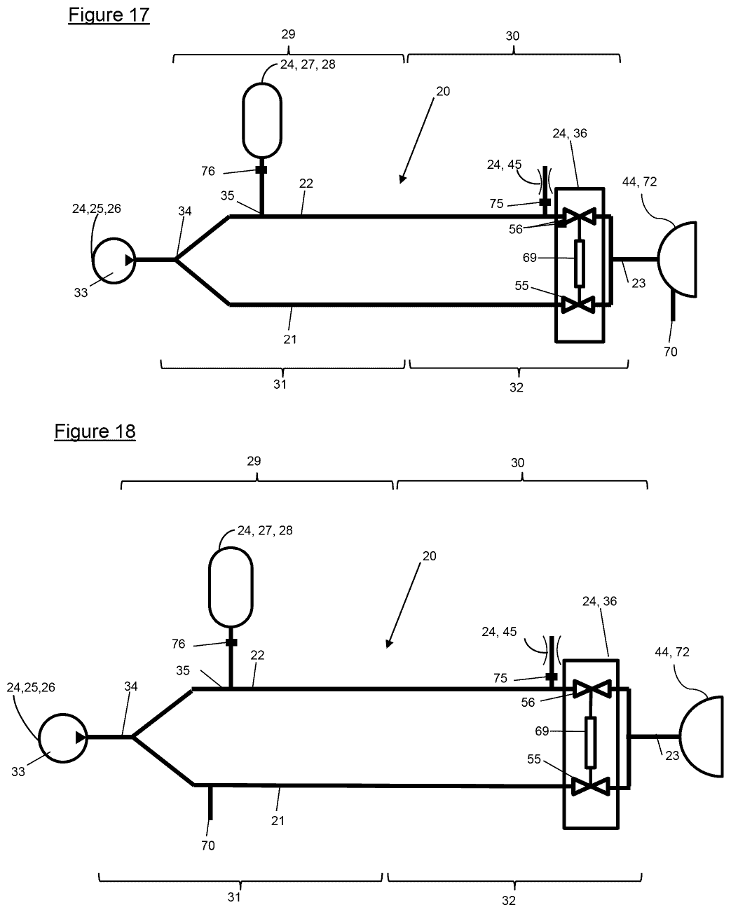

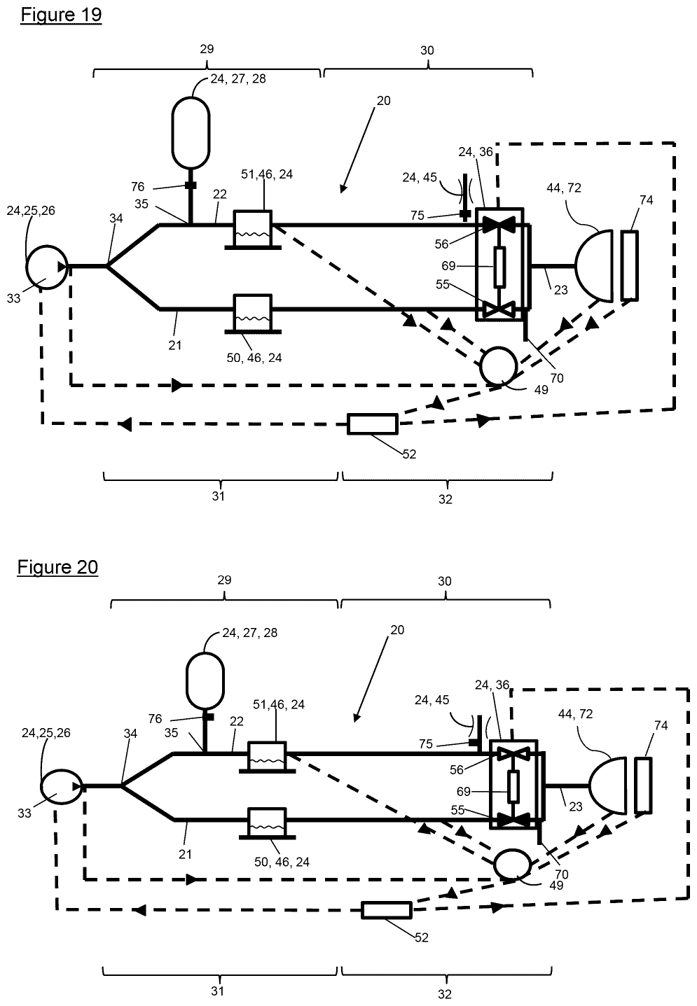

The embodiments shown in FIGS. 1 to 14 can be used for providing respiratory therapy to a patient such as unsealed respiratory therapy. An example includes nasal high flow therapy which can be effective in increasing oxygenation of the patient's blood and/or reducing the work of breathing.

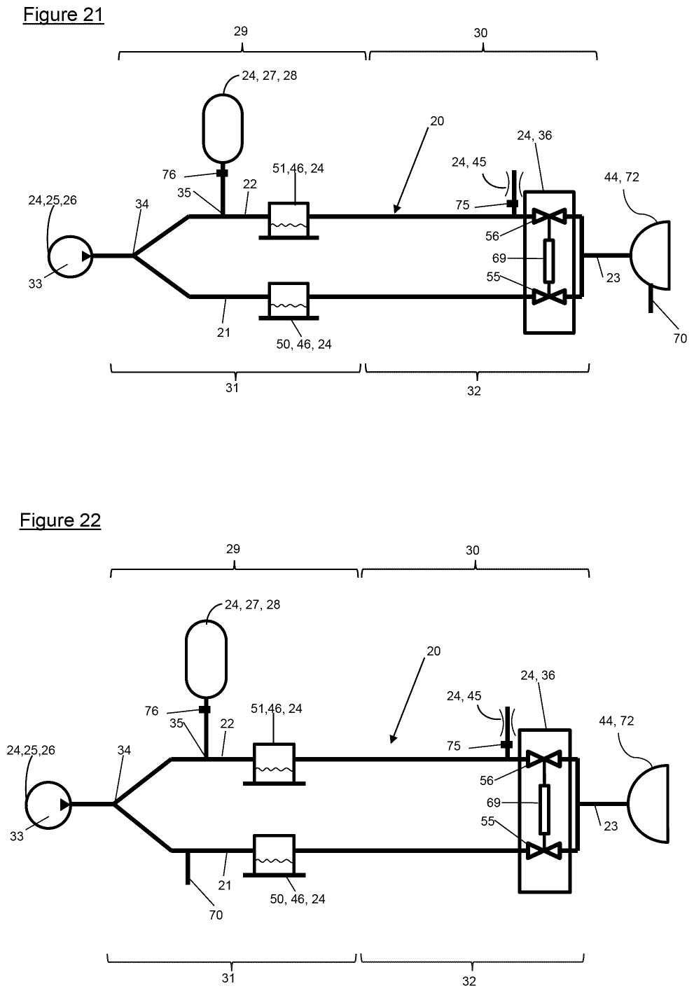

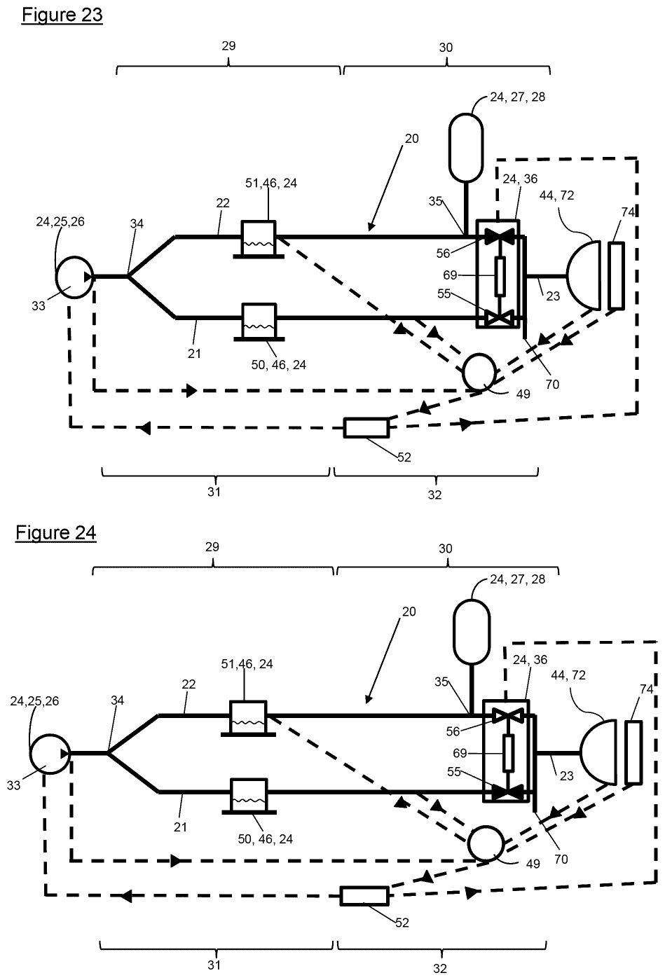

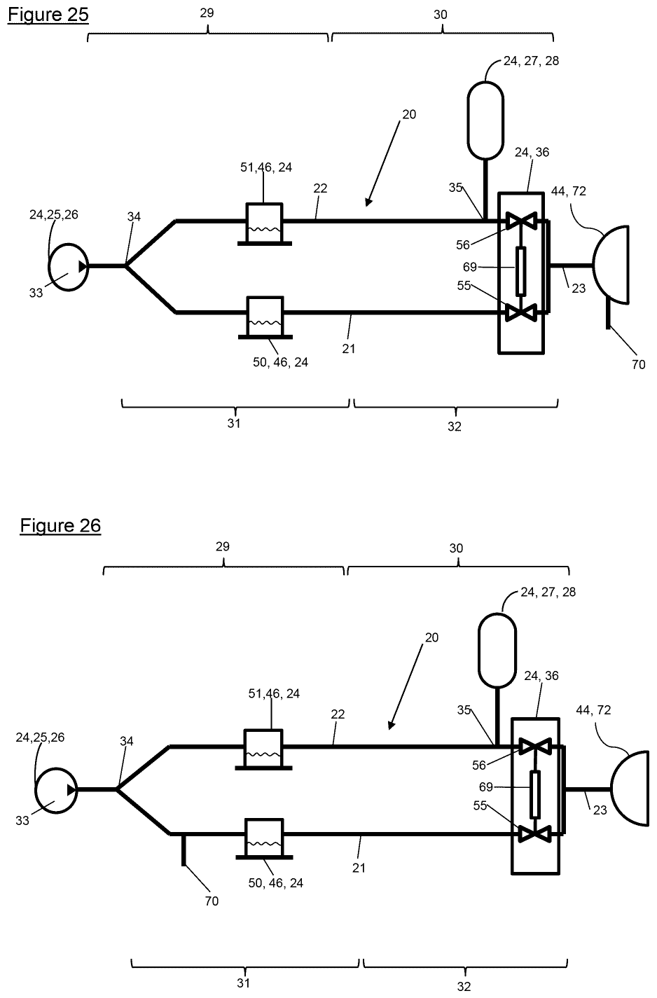

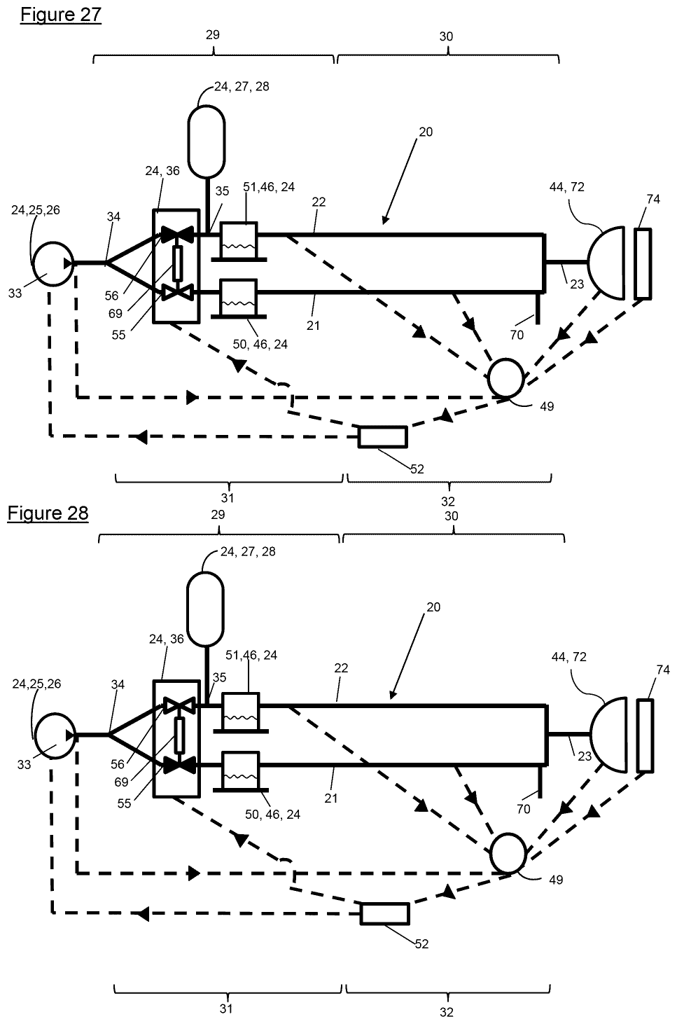

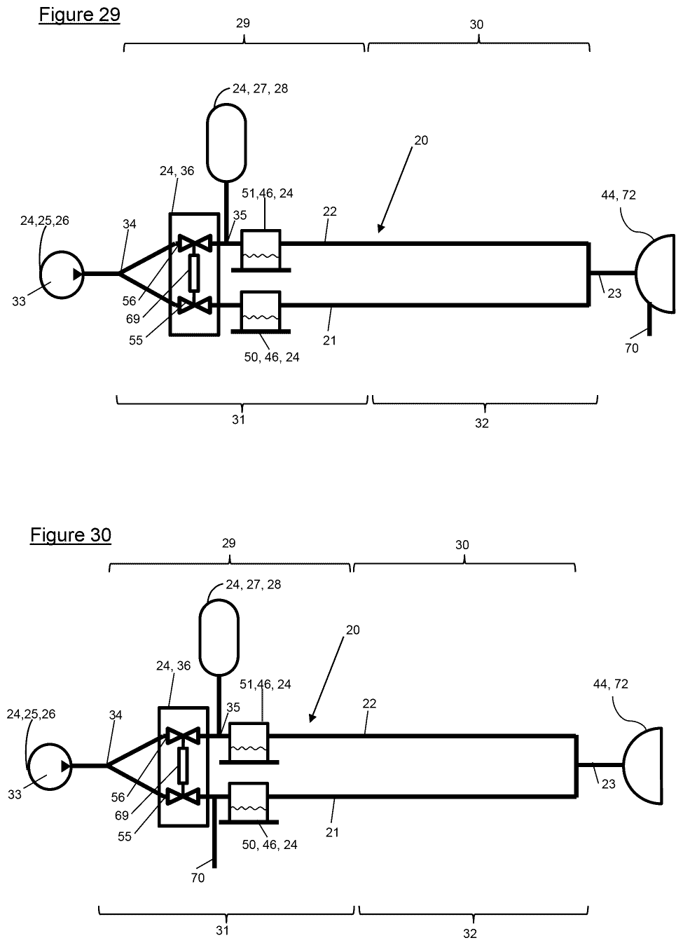

FIGS. 1 and 2 illustrate a breathing circuit 20 including first and second passageways 21, 22 in which distal portions 31, 29 respectively thereof are connected to a first gas source 25, and the distal portion 29 of the second passageway 22 is also connected to a second gas source 27. The breathing circuit 20 has a flow assembly 24 that includes: i) the first gas source 25, suitably a flow generator 33 that blows filtered or unfiltered air as a first gas 26, ii) the second gas source 27, and iii) an active valve mechanism 36 that alternately opens and closes either the first passageway 21 or the second passageway 22 to a patient interface 44.

The flow assembly 24 also includes a first gas inlet 34 on the distal portions 31, 29 of the first and second passageways 21, 22 that connects to an outlet of a flow generator 33. The first gas inlet 34 may be any suitable three limb joiner such as a Y-shaped joiner or a T-shaped joiner. The three limb joiner acts as a splitter to supply the first gas 26 to the first and the second passageways 21, 22. Similarly, the second gas source 27 may be connected to the second passageway 22 at a second gas inlet 35 that may be any suitable three limb joiner.

The active valve mechanism 36 in FIGS. 1 and 2 includes two separate adjustable valves, namely a first valve 55 for the first passageway 21 that is opened in FIG. 1 and closed in FIG. 2, and a second valve 56 for the second passageway 22 that is closed in FIG. 1 and opened in FIG. 2. The opened/closed status of the first and second valves 55, 56 in FIG. 1 represents the first passageway 21 being in fluid communication with the patient interface 44 i.e., opened to the patient interface 44, and the second passageway 22 not being in fluid communication with the patient interface 44 i.e., is closed to the patient interface 44 and is the configuration of the flow assembly 24 and the passageways 21, 22 during patient exhalation. The opened/closed status of the first and second valves 55 and 56 in FIG. 2 represents the second passageway 22 being in fluid communication with the patient interface 44 i.e., opened to the patient interface 44, and the first passageway 21 not being in fluid communication with the patient interface 44, i.e., is closed to the patient interface 44, and is the configuration of the flow assembly 24 and the passageways 21, 22 during patient inhalation. It will also be appreciated that both the first and second valves 55 and 56 may be open for an overlapping period. For example, when the breathing cycle changes from patient inhalation to patient exhalation or vice versa, one of the valves 55 or 56 that is closed may move to an opened position for a period of time before the other valve 55 or 56 that is opened moves to a closed position.

The first and second valves 55 and 56 shown in FIGS. 1 and 2 may be any suitable two port valves having an inlet and an outlet. The first and second valves 55 and 56 may be an actively-controlled valve mechanism 36. The actively-controlled valve mechanism 36 may be adjusted by any suitable actuator 73, such as a solenoid actuator, a pneumatic actuator, an electric motor and so forth. Examples of suitable valves include shuttle valves, spool valves, ball valves, gate valves, butterfly valves, a diaphragm valve switch valves and so forth.

Although not illustrated the first and second valves 55 and 56 may be substituted with a single three-way valve, such as a shuttle valve or a spool valve having one outlet connected to the patient interface 44 and two inlets, one of each being connected to the first and second passageways 21 and 22 respectively. The three-way valve may be an actively-controlled valve mechanism 36 driven by any suitable actuator 73, such as a solenoid actuator, a pneumatic actuator, an electric motor and so forth

During patient exhalation the first gas 26 is supplied simultaneously to the distal portions 31 and 29 of the first and second passageways 21 and 22, as shown in FIG. 1, and the active valve mechanism 36 is configured to allow supply of the first gas 26 to the patient interface 44 via the first passageway 21, suitably a nasal cannula, and disallow supply of the first gas to the patient interface 44 via the second passageway 22. While the first passageway 21 is in fluid communication with the patient interface 44 such as a nasal cannula, the first gas 26, such as filtered air, is supplied to the patient interface 44. The filtered air flushes exhaled gases from the anatomical dead spaces of the patient at the end of the exhalation phase of the patient's breathing cycle. Additionally, high flow therapy can increase expiratory time of the patient 74 due to pressure during expiration. This in turn reduces the respiratory rate of the patient 74.

In addition, the flow assembly 24 includes a vent 45, such as a venting valve, extending from the second passageway 22 upstream of the second valve 56. During patient exhalation, the second gas 28 enters the second passageway 22 and is stored in the second passageway 22. In one example, the second gas 28 is supplied into the second passageway 22 at a slightly higher pressure than the first gas 26, inhibiting the first gas 26 from flowing downstream of the second gas inlet 35 during patient exhalation. Residual breathing gas downstream of a head of the second gas 28 flowing in a direction toward the patient can be vented by the vent 45. In addition, the vent 45 allows the second gas 28 to flow into the second passageway 22 during patient exhalation without increasing the pressure in the second passageway 22 or flowing upstream and entering the first gas passageway 21. The vent 45 may be arranged as any suitable flow restriction including a restriction orifice, a control valve, a positive end expiratory pressure valve (PEEP valve), an aperture of fixed size, or a controlled valve outlet. In one example, the vent 45 is arranged to discharge gas from the second passageway 22 at a rate at which the second gas 28 enters the second passageway 22. If required, a control valve, not illustrated, can be used for regulating the flow of the second gas 28 into the second passageway 22. Similarly, a vent sensor 75 can be used to measure the rate at which gas is discharged from the vent 45, and an output of the flow sensor 75 can be used to operate the control valve 76 to regulate the rate at which the second gas 28 enters the second passageway 22. In one example, the vent 45 may comprise a valve that can close the vent 45 during patient inhalation. That is, the vent 45 may be closed when the second valve 56 is open. This ensures that during patient inhalation, second gas that has accumulated in the second passageway is not exhausted from the vent 45, and/or ambient air is not drawn into the second passageway through vent 45.

Although not shown in FIGS. 1 and 2, the flow assembly 24 may include a non-return valve upstream of the second gas inlet 35, such as between the first gas inlet 34 and the second gas inlet 35 for inhibiting the second gas 28 flowing into the first passageway 21.

At the end of patient exhalation, or at a pause between patient exhalation and patient inhalation, the active valve mechanism 36 switches from the configuration shown in FIG. 1 to the configuration shown in FIG. 2. That is to say, the first gas 26 simultaneously supplied to the first and second passageway is allowed to flow along the second passageway 22 by the second valve 56 being opened, and is inhibited from flowing along the first passageway 21 by the first valve 55 being closed. At least during the initial stages of patient inhalation, the patient 74 receives the second gas 28 that has been stored in the second passageway 22, such as oxygen gas to provide a therapeutic benefit. Specifically, the second gas 28, may comprise high concentrations of the oxygen gas can be drawn into the alveoli of the patient's lungs which in turn, can increase the oxygen levels in the patient's blood. Throughout patient inhalation, the first gas 26 is supplied to the distal portion 29 of the second passageway 22 so that when the second gas 28 stored in the second passageway 22 has been supplied to the patient interface 44 and the patient continues to inhale, the patient will receive a mixture of the first gas 27 and the second gas 28 until inhalation has finished. The second gas 28 may enter the second passageway 22 at a constant rate throughout the breathing cycle of the patient.

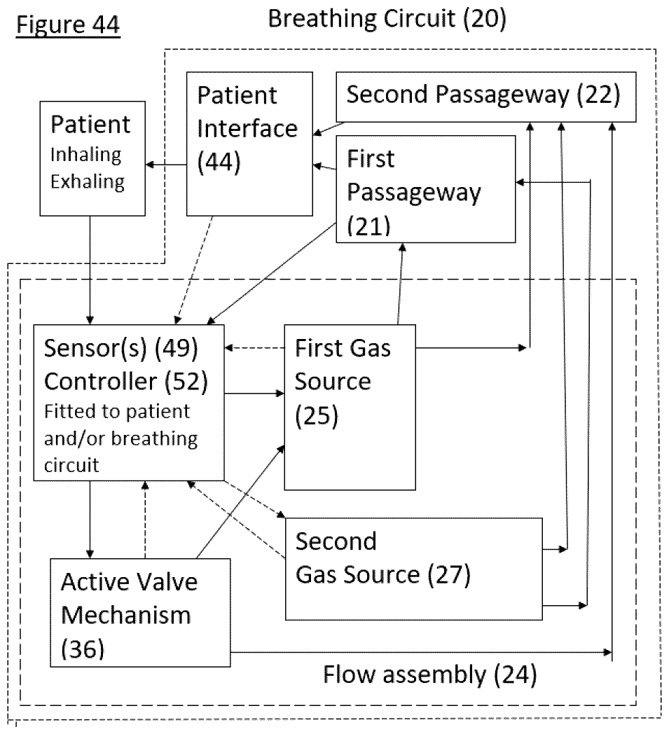

The active valve mechanism 36, and indeed the first and second valves 55 and 56 can change between opened and closed positions in response to outputs of at least one sensor 49 that detects the breathing cycle of the patient, namely when the patient is inhaling and exhaling. The at least one sensor 49 may be located anywhere within the breathing circuit 20 or on the patient, as represented by the dashed line leading to the sensor 49 in FIG. 1. For example, the at least one sensor 49 may be located on either, the patient interface, the first passageway 21, the second passageway 22, on the flow generator 33, or when multiple sensors 49 are provided, the sensors 49 may be provided on any combination of these. The at least one sensor 49 may measure flow rate, temperature, gas pressure, gas composition or any combination of these parameters to detect the breathing cycle of the patient. A controller 52 may receive an output of the sensor 49 which the controller 52 then uses to control operation of either the flow generator 33, or the active valve mechanism 36, or a combination of both. The controller 52 may operate the active valve mechanism 36 to open and close the first and second valves 21 and 22. Further details of the sensor 49 and, optional control for operating the active valve mechanism 36 is described in detail with reference to FIG. 44 below.

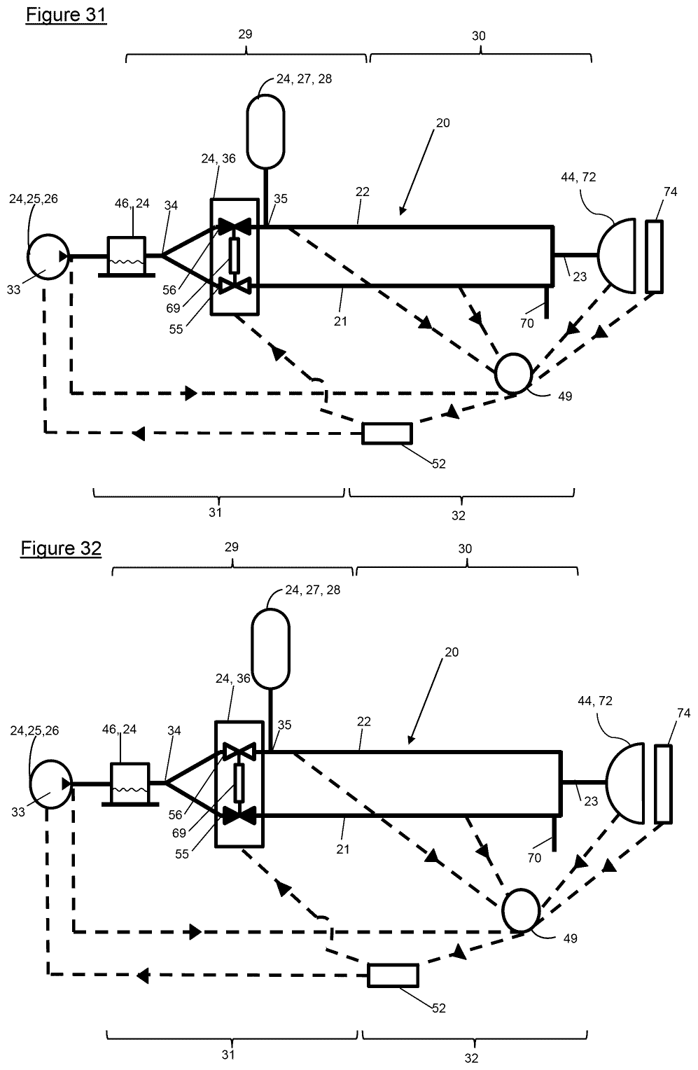

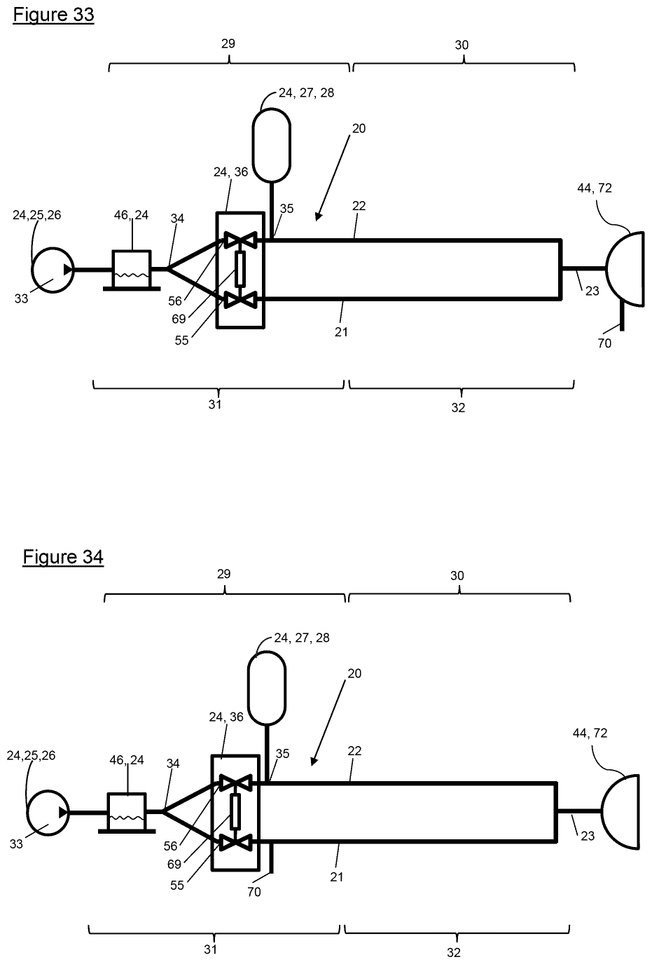

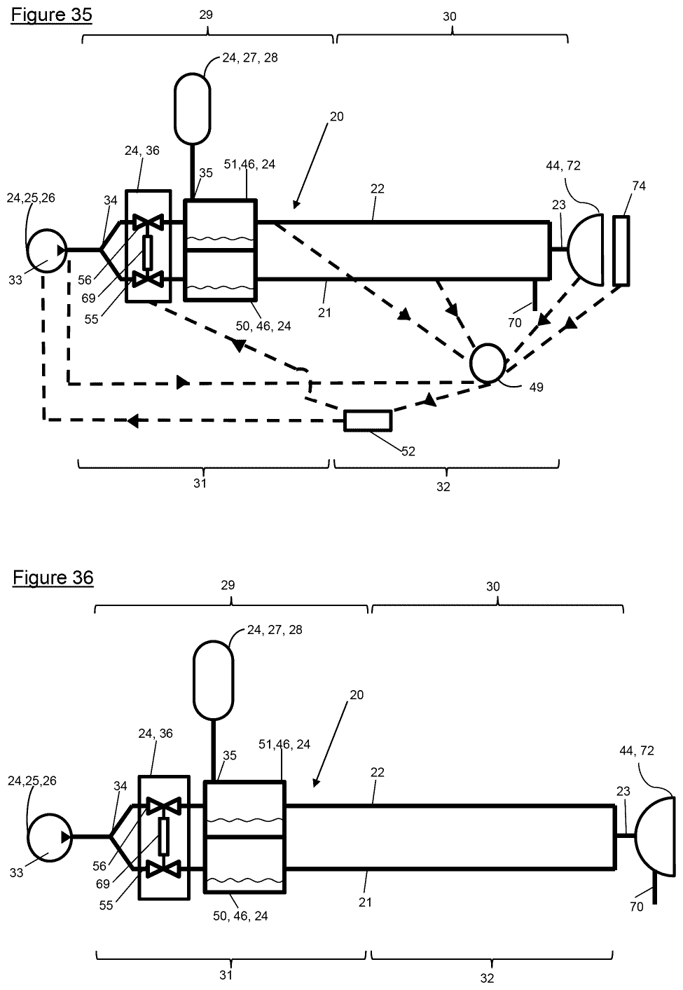

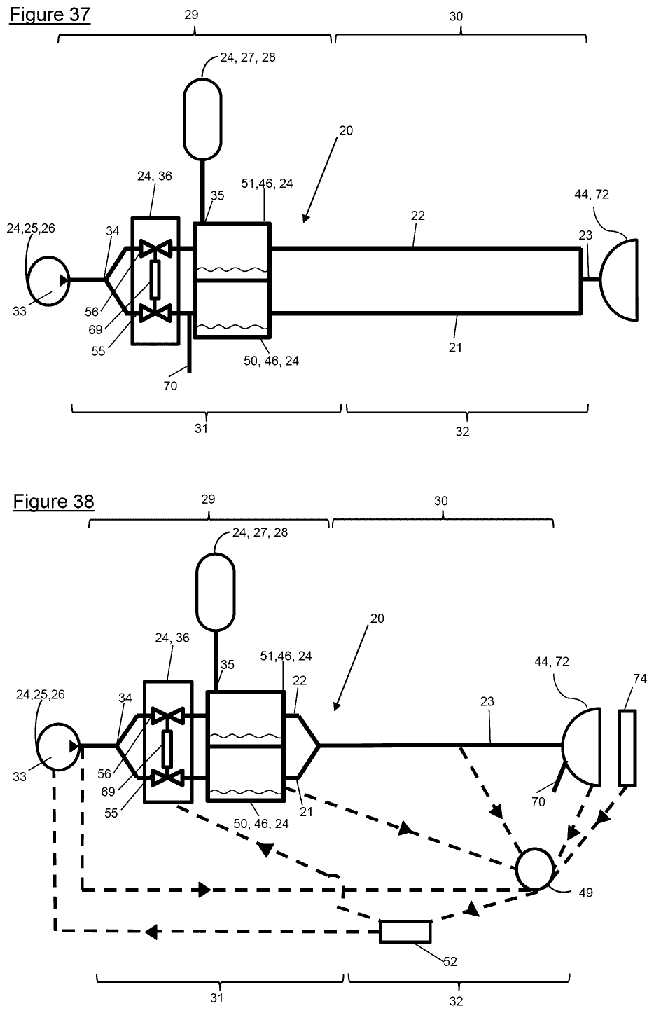

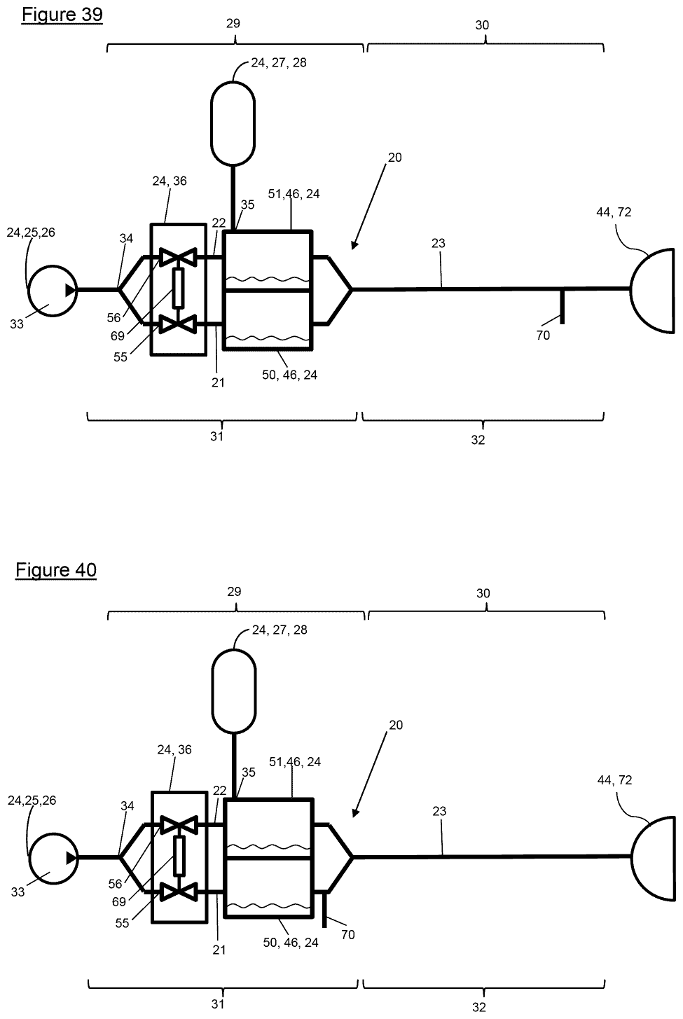

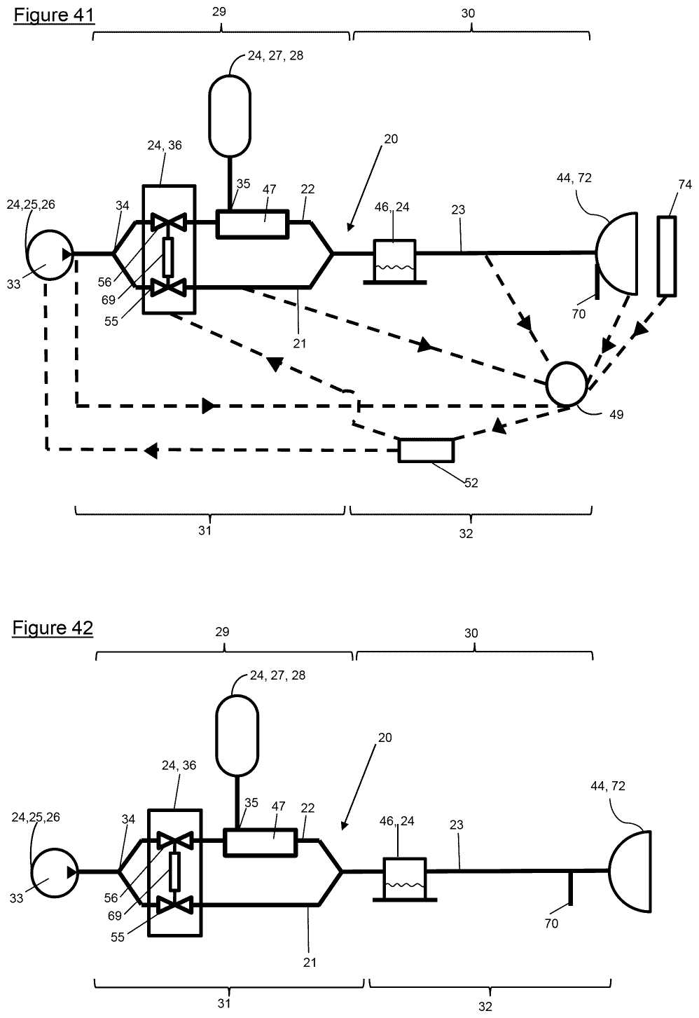

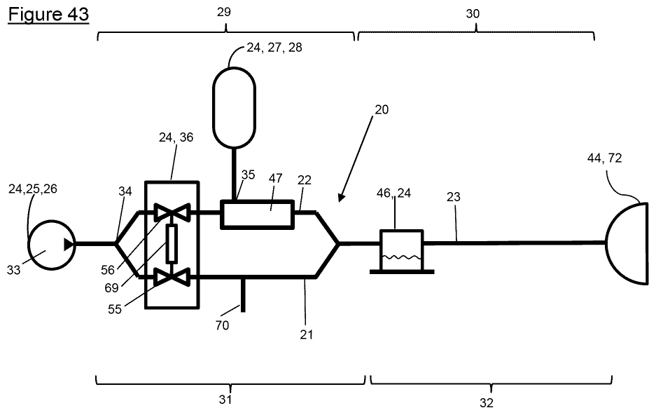

The sensor 49 and the controller 52 can operate the flow generator 33 to provide the required level of flow therapy. As one of the first passageway 21 or the second passageway 22 are opened at any one time to allow flow to the patient interface 44, the controller 52 can operate the active valve mechanism 36 independently of the operation of the flow generator 33. The sensor 49 and controller 52 can also operate the flow generator 33 independently of the flow rate of the second gas 28 that accumulates in the second passageway 22. For instance, a flow sensor (not illustrated) on the vent 45 and a control valve (not illustrated) on the second gas inlet 35 for controlling the flow of the second gas 28 into the second passageway 22 can be operated independently of operation of the flow generator 33. The flow generator 33 may also be controlled so that the flow of the first gas 26 conveyed along the second passageway 22 is reduced during patient inhalation, compared to during patient exhalation. This will take into consideration that the total flow of the breathing gas received by the patient interface 44 during inhalation is a function of the sum of the flow rates of the first gas 26 and the second gas 28 entering the second passageway 22. In one example, operation of the flow generator 33 can be controlled by a flow sensor 28 located at or near the second gas inlet 35 that detects the flow rate of the second gas 28 entering the second passageway 22, and an output from the sensor 49 may then be used by the controller 52 to adjust or reduce the flow rate of the first gas 26 during inhalation by a determined amount to control the total flow rate of the breathing gas being received by the patient interface 44. In another example, operation of the flow generator 33 can be controlled by a flow sensor 49 located on or near the patient interface 44 that detects the flow rate of the breathing gas received by the patient interface 44. An output from the sensor 49 may then be used by the controller 52 to adjust the flow of first gas 26 conveyed along the second passageway 22 during patient inhalation. According to both examples, the output of the sensor 49 can be used by the controller 52 to continually adjust the flow generator 33 so the total flow of the breathing gas received by the patient interface 44 is controlled to the desired amount at any time. The breathing circuit 20 illustrated in FIGS. 3 and 4 is the same as the breathing circuit 20 shown in FIGS. 1 and 2 respectively, save for the flow assembly 24 including dedicated first and second humidification devices 50, 51 for the first and second passageways 21 and 22 respectively. The first humidification device 50 humidifies all of the first gas 26 supplied to the patient interface 44 during exhalation. The second humidification device 51 is located downstream of the second gas inlet 35 and humidifies both the first gas 26 and the second gas 28 flowing along the second passageway 22 toward the patient 74. The internal volume of the second passageway 22 will also include a gas flow path of the humidification device 51.

Although not illustrated, an alternative flow assembly 24 may include the first humidification device 50 in the first passageway 21 and no humidification device in the second passageway 22.

Similarly, another flow assembly 24 may include the second humidification device 51 in the second passageway 22 and no humidification device in the first passageway 21.

FIG. 3 illustrates the breathing circuit 20 with the second passageway 22 closed to the patient interface 44. The sensor 49 for detecting the breathing cycle of the patient 74 and the controller 52 for controlling the flow generator 33 and the active valve mechanism 36 have been omitted from FIG. 4 for convenience.

In FIGS. 1 and 3, the active valve mechanism 36 is located at proximal portions 30 and 32, and the second gas inlet 35 is located at distal portions 29 and 31. In this situation, the vent 45 may be located between the active valve mechanism 36 and the second gas inlet 25, and suitably adjacent to the active valve mechanism 36.

FIGS. 5 and 6 illustrate a breathing circuit 20 similar to the breathing circuit 20 shown in FIGS. 1 and 2, in which distal portions 31 and 29 of first and second passageways 21 and 22 are connected to a first gas source 25, suitably a flow generator 33 that blows filtered or unfiltered air as a first gas 26, and an active valve mechanism 36 that alternately opens and closes either the first passageway 21 or the second passageway 22 to a patient interface 44. FIG. 5 illustrates the first passageway 21 opened and the second passageway closed, and FIG. 6 illustrates the second passageway 22 opened and the second passageway closed. It will also be appreciated that both the first and second valves 55 and 56 may be open for an overlapping period, for example, when the breathing cycle 20 changes from patient inhalation to patient exhalation or vice versa. The description of these features in FIGS. 1 and 2 applies equally to the breathing circuit 20 of the FIGS. 5 and 6.

One difference however is the location of the second gas inlet 35. Specifically, in the case of FIGS. 5 and 6, the second gas inlet 35 is located in a proximal portion 30 of the second passageway 33. During patient exhalation, the first and second passageways 21, 22 and the flow assembly 24 have a configuration as shown in FIG. 5. Specifically, the second valve 56 is closed which inhibits fluid communication from the second passage 22 to the patient interface 44, and the first valve 55 is opened which provides fluid communication from the first passageway 21 to patient interface 44 and allows the first gas 26 to be supplied to the patient interface 44. In addition, during exhalation, the second gas 28 enters the second passageway 22 and flows away from the patient interface 44 toward the distal portion 29 of the second passageway 22, that is to say in an upstream direction relative to the direction of flow during patient inhalation. A volume of the second gas 28 entering the second passageway 22 is stored during patient exhalation, and as a head of the second gas 28 travels away from the patient interface 44, residual gas in the second passageway 22 is displaced toward the first passageway 21. The second gas 28 may enter at a higher pressure than the pressure at which the first gas 26 enters. One possible characteristic of the breathing circuit 20 of FIGS. 5 and 6 is that a vent, as shown in FIG. 1 or 4 is not required which simplifies the structure of the circuit 20. Any residual second gas 28 that flows from the second passageway 22 to the first passageway 21 during patient exhalation can be supplied to the patient interface 44 during patient exhalation via the first passageway 21, thereby providing the patient with potentially higher concentrations of the second gas 28 during patient exhalation.

During patient inhalation, the first and second passageways 21, 22 and the active valve assembly 36 have a configuration as shown in FIG. 6. Specifically, first valve 55 is closed which inhibits fluid communication from the first passage 22 to the patient interface 44, and the second valve is opened which provides fluid communication from the second passageway 21 to patient interface 44 and allows the first gas 26 to flow along the first passageway 21. It will also be appreciated that, both the first and second valves 55 and 56 may be open for an overlapping period. For example, when the breathing cycle changes from patient inhalation to patient exhalation or vice versa, one of the valves 55 or 56 that is closed may move to an opened position for a period of time before the other valve 55 or 56 that is opened moves to a closed position. In any event, the first gas 26 flowing along the second passageway 22 displaces the second gas 28 stored in the second passageway 22 so as to supply the second gas 28 to the patient interface 44. If all of the stored second gas 28 in the passageway has been inhaled and patient inhalation continues, a mixture of the second gas 28 and first gas 26 can be supplied to the patient interface 44. Although not shown in FIG. 5, a control valve can be used to control the flow rate of the second gas 28 through the second gas inlet 35.

With reference to FIG. 5, the breathing circuit 20 includes at least one sensor 49 that detects the breathing cycle of the patient 74, namely when the patient 74 is inhaling and exhaling. The at least one sensor 49 may be located anywhere within the breathing circuit 20 or on the patient 74, as represented by the dashed line leading to the at least sensor 49 in FIG. 5. For example, the at least sensor 49 may be located on either, the patient interface 44, the first passageway 21, the second passageway 22, on the flow generator 33, or when multiple sensors 49 are provided, the sensors 49 may be provided on any combination of these. The at least one sensor 49 may measure flow rate, temperature, gas pressure, gas composition or any combination of these parameters to detect the breathing cycle of the patient. A controller 52 may receive an output of the sensor 49 which the controller 52 then uses to control operation of either the flow generator 33, or the active valve mechanism 36, or a combination of both. A control output signal is generated by the controller 52 which is then received by the flow generator 33, the active valve mechanism 36, or a combination of both. A control output signal is generated by the controller 52 which is received by the flow generator 33, the active valve mechanism 36, or a combination of both. By way of example, the specification of International patent application number PCT/NZ2017/050063 (WO2017200394), entitled “Flow path sensing for flow therapy apparatus”, describes methods of determining breathing phases from sensors in high flow breathing circuits. The same techniques could be implemented with any of the embodiments described herein.

The flow generator 33 may also be controlled so that the flow of the first gas 26 conveyed along the second passageway 22 is reduced during patient inhalation, compared to during patient exhalation. This will take into consideration that the total flow of the breathing gas received by the patient interface 44 during inhalation is a function of the sum of the flow rates of the first gas 26 and the second gas 28 entering the second passageway 22. In one example, operation of the flow generator 33 can be controlled by a flow sensor 28 located at or near the second gas inlet 35 that detects the flow rate of the second gas 28 entering the second passageway 22, and an output from the sensor 49 may then be used by the controller 52 to adjust or reduce the flow rate of the first gas 26 during inhalation by a determined amount to control the total flow rate of the breathing gas being received by the patient interface 44. In another example, operation of the flow generator 33 can be controlled by a flow sensor 49 located on or near the patient interface 44 that detects the flow rate of the breathing gas received by the patient interface 44. An output from the sensor 49 may then be used by the controller 52 to adjust the flow of first gas 26 conveyed along the second passageway 22 during patient inhalation. According to both examples, the output of the sensor 49 can be used by the controller 52 to continually adjust the flow generator 33 so the total flow of the breathing gas received by the patient interface 44 is controlled to the desired amount at any time.

The sensor 49 and the controller 52 can operate the flow generator 33 to provide the required level of flow therapy. The controller 52 can operate the active valve mechanism 36 independently of the operation of the flow generator 33. The sensor 49 and controller 52 can also operate the flow generator 33 independently of the flow rate of the second gas 28 that accumulates in the second passageway 22. The sensor 49 and controller 52 have been omitted from FIG. 6.

FIGS. 5 and 6 also illustrate the flow assembly 24 including dedicated first and second humidification devices 50, 51 in the first and second passageways 21, 22 respectively. The first humidification device 50 humidifies the first gas 26 flowing along the first passageway 21. The second humidification 51 device is located at a distal portion 29 of the second passageway 22 and is spaced from the second gas inlet 35 so that the head of the second gas 28 is not intended to enter the second humidification device 51 during patient exhalation. That is to say, the volume of the second gas 28 stored in a second passageway 22 is intended to be accommodated upstream of the second gas inlet 35 without entering the second humidification device 51.

Alternatively the second humidification device 51 may be located in the proximal portion 30 or in the distal portion 29 (as in, for example, FIGS. 5 and 6) and the head of the second gas 28 may be intended to enter the second humidification device 51. The humidification device therefore provides a greater volume for storing the second gas 28. In this situation, the second humidification device 51 may be located as close as possible to the second gas inlet 35.

If required, either one or both of the first and second humidification devices 50 and 51 illustrated in FIGS. 5 and 6 can be omitted. In addition, in the event that second gas 28 is required to be humidified, a humidification device (not shown) can be located between the source of the second gas 27 and the second gas inlet 35.

FIGS. 7 and 8 illustrate a breathing circuit 20 including first and second passageways 21, 22 both having proximal portions 32 and 30 connected to a patient interface 44, and a flow assembly 24 having an active valve mechanism 36 in which distal portions 31 and 29 of the first passageway 21 and the second passageway 22 are alternately opened and closed to a first gas source 25, suitably in the form of a flow generator 33 blowing filtered or unfiltered air as a first gas 26. FIG. 7 illustrates the flow assembly 24 in which the first passageway 21 is opened to the first gas source 25 during patient exhalation and the second passageway 22 is not in fluid communication with the first gas source 25. FIG. 8 illustrates the flow assembly 24 in a configuration in which the second passageway 22 is in fluid communication with the first gas source 25, and the first passageway 21 is not in fluid communication with the first gas source 25. That is to say, FIG. 7 illustrates the first passageway 21 opened and the second passageway closed, and FIG. 8 illustrates the second passageway 22 opened and the second passageway closed.

The active valve mechanism 36 in FIGS. 7 and 8 includes two separate adjustable valves, namely a first valve 55 for the first passageway 21 that is opened in FIG. 7 and closed in FIG. 8, and a second valve 56 for the second passageway 22 that is closed in FIG. 7 and opened in FIG. 8. Specifically, the opened/closed status of the first and second valves 55 and 56 in FIG. 7 represents the first passageway 21 being in fluid communication with the first gas source 25 so that the first gas can flow along the first passageway 21, and the second passageway 22 not being in fluid communication with the first gas source 25, i.e., is closed to the first gas source 25. Similarly, the opened/closed status of the first and second valves 55 and 56 in FIG. 2 represents the second passageway 22 being in fluid communication with the first gas source 25 i.e., opened to the first gas source 25, and the first passageway 21 not being in fluid communication with the first gas source 25, i.e., is closed to the first gas source 25. It will also be appreciated that both the first and second valves 55 and 56 may be open for an overlapping period. For example, when the breathing cycle changes from patient inhalation to patient exhalation or vice versa, one of the valves 55 or 56 that is closed may move to an opened position for a period of time before the other valve 55 or 56 that is opened moves to a closed position.

Although not illustrated in FIGS. 7 and 8, the active valve mechanism 36 may be an actively-controlled valve. The actively-controlled valve may be, for example, adjusted by any suitable actuator, such as a solenoid actuator, a pneumatic actuator, an electric motor and so forth. Examples of suitable valves include shuttle valves, spool valves, ball valves, gate valves, butterfly valves, a diaphragm valve switch valves and so forth. In another example, the active valve mechanism 36 may also be a single three-way valve, such as a shuttle valve or a spool valve having one inlet for connection to the output of the flow generator 33 and two outlets, one of each outlet being connected to the first and second passageways 21 and 22 respectively.

Referring to FIG. 7, during patient exhalation, the first valve 55 is opened and the first gas 26 flows from the first gas source 25 along the first passageway 21 to supply the first gas 26 to the patient interface 44. In addition, the second valve 56 is closed and the second gas 28 enters the second gas passageway 22 at the second gas inlet 35 located in the distal portion 29 so that a head of the second gas 28 flows in a direction toward the patient interface 44 to store a volume of the second gas 28 in the second passageway 22. Residual gases in the second passageway 22 downstream of the head of the second gas 28 are also supplied to the patient interface 44. The second gas 28 is supplied at a slightly higher pressure than the first gas 26 so that the first gas 26 is inhibited from passing from the first passageway 21 to the second passageway 22 during patient exhalation. If required, a non-return valve, not illustrated, can be provided in the second passageway 22 to inhibit flow in a direction from a proximal portion 30 toward a distal portion 29.

Referring to FIG. 8, during patient inhalation the second valve 56 is opened, and the first gas 28 flows form the first gas source 25 to and along the second passageway 22 to supply the first gas 26 upstream of the second gas inlet 35. At the start of patient inhalation, the first gas 26 flowing along the second passageway 22 displaces the second gas 28 stored in the second passageway 22 so as to supply the second gas 28 to the patient interface 44. If all of the stored second gas 28 in the passageway has been inhaled and patient inhalation continues to inhale, a mixture of the second gas 28 and first gas 26 can be supplied to the patient interface 44. In one example the second gas 28 enters the second passageway 22 at a constant rate throughout the breathing cycle. However, if required, the flow rate of the second gas 28 can be controlled, for example, by a control valve to adjust the flow of the second gas 28 through the second gas inlet 35.

With reference to FIG. 7, the breathing circuit 20 includes at least one sensor 49 that detects the breathing cycle of the patient 74, namely when the patient is inhaling and exhaling. The at least one sensor 49 may be located anywhere within the breathing circuit 20 or on the patient 74, as represented by the dashed line leading to the sensor 49 in FIG. 7. For example, the at least sensor 49 may be located on either, the patient interface 44, the first passageway 21, the second passageway 22, on the flow generator 33, or when multiple sensors 49 are provided, the sensors 49 may be provided on any combination of these. The at least one sensor 49 may measure flow rate, temperature, gas pressure, gas composition or any combination of these parameters to detect the breathing cycle of the patient. A controller 52 may receive an output of the sensor 49 which the controller 52 then uses to control operation of either the flow generator 33, or the active valve mechanism 36, or a combination of both. By way of example, the specification of International patent application number PCT/NZ2017/050063 (WO2017200394), entitled “Flow path sensing for flow therapy apparatus”, describes methods of determining breathing phases from sensors in high flow breathing circuits. The same techniques could be implemented with the embodiments described herein.

The sensor 49 and the controller 52 can operate the flow generator 33 to provide the required level of flow therapy. The controller 52 can operate the active valve mechanism 36 independently of the operation of the flow generator 33. The sensor 49 and controller 52 can also operate the flow generator 33 independently of the flow rate of the second gas 28 that accumulates in the second passageway 22. The flow generator 33 may also be controlled so that the flow of the first gas 26 conveyed along the second passageway 22 is reduced during patient inhalation, compared to during patient exhalation. This will take into consideration that the total flow of the breathing gas received by the patient interface 44 during inhalation is a function of the sum of the flow rates of the first gas 26 and the second gas 28 entering the second passageway 22. In one example, operation of the flow generator 33 can be controlled by a flow sensor 28 located at or near the second gas inlet 35 that detects the flow rate of the second gas 28 entering the second passageway 22, and an output from the sensor 49 may then be used by the controller 52 to adjust or reduce the flow rate of the first gas 26 during inhalation by a determined amount to control the total flow rate of the breathing gas being received by the patient interface 44. In another example, operation of the flow generator 33 can be controlled by a flow sensor 49 located on or near the patient interface 44 that detects the flow rate of the breathing gas received by the patient interface 44. An output from the sensor 49 may then be used by the controller 52 to adjust the flow of first gas 26 conveyed along the second passageway 22 during patient inhalation. According to both examples, the output of the sensor 49 can be used by the controller 52 to continually adjust the flow generator 33 so the total flow of the breathing gas received by the patient interface 44 is controlled to the desired amount at any time. The sensor 49 and controller 52 have been omitted from FIG. 8.

As can be seen, the flow assembly 24 also includes dedicated first and second humidification devices 50, 51 in the first and second passageways 21, 22. The first humidification device 50 humidifies the first gas 26 conveyed by the first passageway 21. The second humidification device 51 is located downstream of the second gas inlet 35 and humidifies the second gas 28 entering during patient exhalation, and humidifies the first gas 26 and the second gas 28 flowing along the second gas passageway 22 during patient inhalation.

If required, either one of the first and second humidification devices 50 and 51 may be omitted or removable from the breathing circuit 20.

Although not illustrated in FIGS. 7 and 8, the second gas inlet 35 may be located at a proximal portion 30 of the second gas passageway 22. In this instance, a vent will be required to discharge any residual gas in the second passageway 22 as the second gas 28 is stored therein during patient exhalation. The vent may be located at a distal portion of the second passageway 22. In addition, a valve such as non-return valve or an actively controlled valve may be located between the second gat inlet 35 and the patient interface 44.

FIGS. 9 and 10 illustrate a breathing circuit 20 that is the same as the breathing circuit 20 shown in FIGS. 7 and 8, save for the first and second humidification devices 50 and 51 being replaced with a single humidification device 46 located downstream of the first gas source 25 and upstream of the first and second passageways 21 and 22. Suitably the humidification device 46 is located between the outlet of the first gas source 25 and upstream of the active valve mechanism 36. In this circuit 20, the first gas 26 supplied to the patient interface 44 will be humidified, whereas the second gas 28 will not be separately humidified. If required, a further humidification device may be provided to humidify the second gas 28 prior to being supplied to the second gas inlet 35. Although not illustrated, it also possible that the flow generator 33 and the humidification device 50 may be integrated together, or connected together so as to effectively form a single equipment item.

In FIG. 9, the first valve 55 is opened and the second valve 56 closed so that first passageway 21 is in flow connected to the patient interface 44. In FIG. 10, the second passageway 22 is opened to the patient interface 44. It will also be appreciated that both the first and second valves 55 and 56 may be open for an overlapping period. For example, when the breathing cycle changes from patient inhalation to patient exhalation or vice versa, one of the valves 55 or 56 that is closed may move to an opened position for a period of time before the other valve 55 or 56 that is opened moves to a closed position.

With reference to FIG. 9, the breathing circuit 20 includes at least one sensor 49 that detects the breathing cycle of the patient 74, namely when the patient is inhaling and exhaling. The at least one sensor 49 may be located anywhere within the breathing circuit 20 or on the patient 74, as represented by the dashed line leading to the sensor 49 in FIG. 9. For example, the at least sensor 49 may be located on either, the patient interface 44, the first passageway 21, the second passageway 22, on the flow generator 33, or when multiple sensors 49 are provided, the sensors 49 may be provided on any combination of these. In addition to gas flow rate, the sensor 49 may measure temperature, gas pressure or gas composition to detect the breathing cycle of the patient 74. A controller 52 may receive an output of the sensor 49 which the controller uses to control operation of either the flow generator 33, or the active valve mechanism 36, or a combination of both. By way of example, the specification of International patent application number PCT/NZ2017/050063 (WO2017200394), entitled “Flow path sensing for flow therapy apparatus”, describes methods of determining breathing phases from sensors in high flow breathing circuits. The techniques described therein could be implemented with the present embodiments.

The sensor 49 and the controller 52 can operate the flow generator 33 to provide the required level of flow therapy. However, as one of the first passageway 21 or the second passageway 22 are opened at any one time to allow flow to the patient interface 44, the controller 52 can operate the active valve mechanism 36 independently of the operation of the flow generator 33. The sensor 49 and controller 52 can also operate the flow generator 33 independently of the flow rate of the second gas 28 that accumulates in the second passageway 22. The flow generator 33 may also be controlled so that the flow of the first gas 26 conveyed along the second passageway 22 is reduced during patient inhalation, compared to during patient exhalation. This will take into consideration that the total flow of the breathing gas received by the patient interface 44 during inhalation is a function of the sum of the flow rates of the first gas 26 and the second gas 28 entering the second passageway 22. In one example, operation of the flow generator 33 can be controlled by a flow sensor 28 located at or near the second gas inlet 35 that detects the flow rate of the second gas 28 entering the second passageway 22, and an output from the sensor 49 may then be used by the controller 52 to adjust or reduce the flow rate of the first gas 26 during inhalation by a determined amount to control the total flow rate of the breathing gas being received by the patient interface 44. In another example, operation of the flow generator 33 can be controlled by a flow sensor 49 located on or near the patient interface 44 that detects the flow rate of the breathing gas received by the patient interface 44. An output from the sensor 49 may then be used by the controller 52 to adjust the flow of first gas 26 conveyed along the second passageway 22 during patient inhalation. According to both examples, the output of the sensor 49 can be used by the controller 52 to continually adjust the flow generator 33 so the total flow of the breathing gas received by the patient interface 44 is controlled to the desired amount at any time. The sensor 49 and controller 52 have been omitted from FIG. 10.

Although not illustrated in FIGS. 9 and 10, a non-return valve may be located in a proximal portion 30 of the second passageway 22 to inhibit the first gas 26 from passing from the first passageway 21 to the second passageway 22 during patient exhalation.

Similarly, although not illustrated in the figures, the active valve mechanism 36 may be located at the proximal portion 31 and 29 of the first and second passageways 21 and 22 similar to the breathing circuit 20 illustrated in FIGS. 1 to 6. In this situation a non-return valve may be located at a distal portion 29 of the second passageway 22 to inhibit the second gas 28 from entering the first passageway 21. In this instance, a vent (not shown) is required in the proximal portion 30 of the second passageway 22.

Another modification is the second gas inlet 35 may be located in the proximal portion 30 of the second passageway 22. In this instance, a vent (not shown) will be required to discharge any residual gas in the second passageway 22 as the second gas 28 is stored therein during patient exhalation. The vent may be located at a distal portion of the second passageway.

In another modification, the active valve mechanism 36 and the second gas inlet 35 may be located in the proximal portion 30 of the second passageway 22. This modification avoids the need for a vent in the second passageway 22.

FIG. 11 illustrates a breathing circuit 20 that is much the same as the breathing circuit 20 shown in FIGS. 7 and 8 in which proximal portions 32 and 30 of the first and second passageways 21 and 22 are connected to the patient interface 44. The active valve mechanism 36 shown in FIG. 11 is an example of first and second control valves 43 located in the distal portions 31 and 29 of the first and second passageways 21 and 22, respectively. The first and second valves 55, 56 shown in FIG. 11 may be any suitable two port valves having an inlet and an outlet. The first and second valves 55, 56 may be actively adjusted by any suitable actuator, such as a solenoid actuator, a pneumatic actuator, an electric motor and so forth. Each control valve 43 has two ports, and in which one valve is opened while the other is closed, and vice versa, so that flow in the first and a second passageways 21 and 22 alternates depending on whether the patient in inhaling or exhaling. Although FIG. 11 illustrates the first and second valves 55 and 56 in the opened position, only one of the first and second valves 55 and 56 would be opened at any time during use of the breathing circuit 10. Specifically, during patient exhalation, the first valve 55 is opened and the second valve 56 is closed so as to supply the first gas 26 to the patient interface 44 via the first passageway 21. It will be appreciated that both the first and second valves 55 and 56 may be opened for an overlapping period. For example, when the breathing cycle changes from patient inhalation to patient exhalation or vice versa, one of the valves 55 or 56 that is closed may move to an opened position for a period of time before the other valve 55 or 56 that is opened moves to a closed position. In any event, the second gas 28 enters the second passageway 22 so that a volume of the second gas 28 is stored in the second passageway 22 so a head of the second gas 28 moves toward the patient interface 44, and residual gas downstream of the head of the second gas 28 can be supplied to the patient interface 44. During patient inhalation, the second valve 56 is opened and the first valve 55 is closed so that the second gas 28 stored in the second passageway 22 is received into the patient's lungs and when the stored second gas 28 has been consumed and the patient continues to inhale, a mixture of the first and second gases 26 and 28 will be received by the patient. The second gas 28 may be supplied at a constant rate to the second passageway 22 during inhalation and exhalation.

In addition, the breathing circuit 20 includes a single humidification device 46 having first and second chambers that form part of the first and second passageways 21 and 22 respectively. The single humidification device 46 may include a dividing wall that essentially defines two isolated flow paths through each chamber so that the gases 26 and 28 in each chamber cannot mix. The volume of the chamber that forms part of the second passageway 22 will contribute to the internal volume for storing the second gas 28. The flow path of the second chamber may have a volume in the range of 140 ml to 580 ml.

The humidification device 46 will also have a first chamber comprising an inlet and outlet for connection to the first passageway 21, and a second chamber comprising an inlet and outlet for connection to the second passageway 22.

The second gas inlet 35 may be connected directly to the second chamber of the humidification device 46 as illustrated, or the second gas inlet 35 may be located upstream of the humidification device 46. When connected to the humidification device 46, a volume of the second gas 28 can be stored in the second chamber of the humidification device during patient exhalation. An amount of the second gas may also be stored in the second passageway 22. In any event, the second chamber of the humidification device 46 may be regarded as forming part of the second passageway 22, or located in the second passageway 22.

The breathing circuit 20 includes at least one sensor 49 that detects the breathing cycle of the patient, namely when the patient is inhaling and exhaling. The at least one sensor 49 may be located anywhere within the breathing circuit 20 or on the patient 74, as represented by the dashed line leading to the sensor 49 in FIG. 9. For example, the at least sensor 49 may be located on either, the patient interface 44, the first passageway 21, the second passageway 22, on the flow generator 33, or when multiple sensors 49 are provided, the sensors 49 may be provided on any combination of these. In addition to gas flow rate, the sensor 49 may measure temperature, gas pressure or gas composition to detect the breathing cycle of the patient 74. A controller 52 may receive an output of the sensor 49 to control operation of either the flow generator 33, or the active valve mechanism 36, or a combination of both. By way of example, the specification of International patent application number PCT/NZ2017/050063 (WO2017200394), entitled “Flow path sensing for flow therapy apparatus”, describes methods of determining breathing phases from sensors in high flow breathing circuits. The same techniques could be implemented with any of the embodiments described herein.

The sensor 49 and the controller 52 can operate the flow generator 33 to provide the required level of flow therapy. The controller 52 can operate the active valve mechanism 36 independently of the operation of the flow generator 33. The sensor 49 and controller 52 can also operate the flow generator 33 independently of the flow rate of the second gas 28 that accumulates in the second passageway 22. The flow generator 33 may also be controlled so that the flow of the first gas 26 conveyed along the second passageway 22 is reduced during patient inhalation, compared to during patient exhalation. This will take into consideration that the total flow of the breathing gas received by the patient interface 44 during inhalation is a function of the sum of the flow rates of the first gas 26 and the second gas 28 entering the second passageway 22. In one example, operation of the flow generator 33 can be controlled by a flow sensor 28 located at or near the second gas inlet 35 that detects the flow rate of the second gas 28 entering the second passageway 22, and an output from the sensor 49 may then be used by the controller 52 to adjust or reduce the flow rate of the first gas 26 during inhalation by a determined amount to control the total flow rate of the breathing gas being received by the patient interface 44. In another example, operation of the flow generator 33 can be controlled by a flow sensor 49 located on or near the patient interface 44 that detects the flow rate of the breathing gas received by the patient interface 44. An output from the sensor 49 may then be used by the controller 52 to adjust the flow of first gas 26 conveyed along the second passageway 22 during patient inhalation. According to both examples, the output of the sensor 49 can be used by the controller 52 to continually adjust the flow generator 33 so the total flow of the breathing gas received by the patient interface 44 is controlled to the desired amount at any time.

Although not shown in FIG. 11, the flow assembly 24 may include a non-return valve in the proximal portion 30 of the second passageway 22 to inhibit the first gas 26 from entering the second passageway 22.

In another configuration, the active valve mechanism 36 of the breathing circuit 20 shown in FIG. 6 can be located in proximal portions 32 and 30 of the first and second passageways 21 and 22, much like the breathing circuit 20 shown in FIGS. 1 to 6.

In another configuration, the second gas inlet 35 can be located in the proximal portion 30 of the second passageway 22. This configuration is possible with the active valve mechanism 36 located either in the proximal portion 32 and 30 or the distal portion 31 and 29 of the passageways 21 and 22. In any event, when the second gas inlet 35 is located in the proximal portion 30 of the second passageway 22, a vent, such as a venting valve (not illustrated) may be required in the distal portion 29 of the second passageway 22 to allow residual gases to be vented during patient exhalation and, in turn, allow a volume of the second gas 28 to be stored in the second passageway 22.

FIG. 12 illustrates a breathing circuit 20 similar to the circuit 20 shown in FIGS. 7, 8 and 11 but in which the first and second passageways 21 and 22 comprise respective distal portions 31 and 29 only. An active valve mechanism 36 is provided in the distal portions 31 and 29 of the passageways 21 and 22, and the first gas source 25 supplies the first gas 26 to the passageways 21 and 22. A second gas inlet 35 is connected to a second chamber of a dual chamber humidification device 46. These features and others of the breathing circuit 20 shown in FIG. 12 can be equated to the features shown in FIGS. 7, 8 and 11.

As can be seen, the first and second passageways 21, 22 merge together to provide a third passageway 23 having a proximal portion that connects to a patient interface 44.

Although FIG. 12 illustrates the first and second valves 55 and 56 in the opened position, in most instances only one of the first and second valves 55 and 56 would be opened at any time during use of the breathing circuit 20. However, it will also be appreciated that both the first and second valves 55 and 56 may be open for an overlapping period. For example, when the breathing cycle changes from patient inhalation to patient exhalation or vice versa, one of the valves 55 or 56 that is closed may move to an opened position for a period of time before the other valve 55 or 56 that is opened moves to a closed position. In most instances, during patient exhalation, the first valve 55 is opened and the second valve 56 is closed so as to supply the first gas 26 to the patient interface 44 via the first passageway 21. During patient inhalation, the second valve 56 is opened and the first valve 55 is closed so that the second gas 28 stored in the second passageway 22 is received into the patient's lungs and when the stored second gas 28 has been consumed and the patient continues to inhale, a mixture of the first and second gases 26 and 28 will be received by the patient. The second gas 28 may be supplied at a constant rate to the second passageway 22 during inhalation and exhalation.

If the active valve mechanism 36 is operated to open the second valve 56 at the start of patient inhalation, residual gases in the third passageway 23 may delay the onset of the stored second gas 28 reaching the patient. The delay may be proportional to the volume of the third passageway 23 and may be minimised by a controller 52 as described below in the following two paragraphs. In the case of FIG. 12, the third passageway 23 will have a length that minimises weight or pull on the patient interface 44.

As the internal volume of the third passageway 23 may be a noticeable portion of the tidal volume of the patient, it may be desirable to control the second valve 56 to compensate for this delay. That is, if the respiratory rate is known, the beginning of inhalation can be predicted, and the active valve mechanism can be operated to start flow of the first gas 26 to the second passageway 22 for a period prior to patient inhalation. The first gas 26 can then reach the patient as inspiration begins. To help take this into account, the breathing circuit 20 includes at least one sensor 49 that detects the breathing cycle of the patient 74, namely when the patient 74 is inhaling and exhaling.

The at least one sensor 49 may be located anywhere within the breathing circuit 20 or on the patient 74, as represented by the dashed line leading to the sensor 49 in FIG. 12. For example, the at least sensor 49 may be located on either, the patient interface 44, the first passageway 21, the second passageway 22, on the flow generator 33, or when multiple sensors 49 are provided, the sensors 49 may be provided on any combination of these. The at least one sensor 49 may measure flow rate, temperature, gas pressure, gas composition or any combination of these parameters to detect the breathing cycle of the patient. The controller 52 may receive an output signal of the sensor(s) 49 which the controller 52 uses to calculate, or has a process that calculates, the period of inhalation and/or exhalation, and in turn, produces a control output signal that is used to operate the active valve mechanism 36. Alternatively, the sensor(s) 49 may determine whether the patient is inhaling or exhaling and the controller 52 produces a control output signal that is used to operate the active valve mechanism 36 to supply the first gas 26 or the second gas 28 depending on the output signal of the sensor(s) 49. In any event, the controller 52 may calculate the period of inhalation, and generate a control output signal that is used to operate the active valve mechanism 36 that minimizes the delay between the active valve mechanism 36 supplying the first gas 26 to the second passageway 22 and the patient receiving the breathing gas from the second passageway 22. In other words, the internal volume of the third passageway 23 and the humidification device 46, if present, can delay the onset of the second gas 28 being supplied to the patient, and the controller 52 can reduce the delay by generating an output signal to operate the active valve mechanism 36 to pre-empt patient breathing. In addition, the flow generator 33 may also be controlled so that the flow of the first gas 26 conveyed along the second passageway 22 is reduced during patient inhalation, compared to during patient exhalation. This will take into consideration that the total flow of the breathing gas received by the patient interface 44 during inhalation is a function of the sum of the flow rates of the first gas 26 and the second gas 28 entering the second passageway 22 as described above in the relation to FIGS. 1 to 11.

FIG. 13 illustrates a breathing circuit 20 that is similar to the circuit 20 shown in FIGS. 7, 8 and 12 in which distal portions 32 and 30 includes first and second passageways 21 and 22 that are connected to a first gas source 25 such as air flow generator 33 as illustrated, and an active valve mechanism 36 including two separate control valves 55 and 56 is provided in the distal portions 31 and 29 of the passageways 21 and 22. These features and others of the circuit 20 shown in FIG. 13 can be equated to the features shown in FIGS. 7, 8 and 12.

Although FIG. 13 illustrates the first and second valves 55 and 56 in the opened position, however in most instances only one of the first and second valves 55 and 56 would be opened at any time during use of the breathing circuit 20. However, it will also be appreciated that both the first and second valves 55 and 56 may be open for an overlapping period. For example, when the breathing cycle changes from patient inhalation to patient exhalation or vice versa, one of the valves 55 or 56 that is closed may move to an opened position for a period of time before the other valve 55 or 56 that is opened moves to a closed position. In most instances during patient exhalation, the first valve 55 is opened and the second valve 56 is closed so as to supply the first gas 26 to the patient interface 44 via the first passageway 21. During patient inhalation, the second valve 56 is opened and the first valve 55 is closed so that the second gas 28 stored in the second passageway 22 is received into the patient's lungs and when the stored second gas 28 has been consumed and the patient continues to inhale, a mixture of the first and second gases 26 and 28 will be received by the patient. The second gas 28 may be supplied at a constant rate to the second passageway 22 during inhalation and exhalation. It is also possible that the second gas 28 could be supplied at a variable rate to the second passageway 22.

As can be seen, the first and second passageways 21 and 22 merge together to provide a third passageway 23 having a proximal portion that connects to a patient interface 44. The flow assembly includes a humidification device 46 located on the third passageway 23 such that all of the breathing gas supplied to the patient interface 44 is substantially humidified uniformly.

The flow assembly 24 also includes a reservoir 47 located in the first passageway 21 to provide the first passageway 21 with the required internal volume. Although not illustrated in the figures, the internal volume of the reservoir 47 may be adjustable, which in turn adjusts the volume of the second gas 28 stored in the second passageway 22 during patient exhalation. Adjustments made to the volume of the reservoir 47 may also need to be taken into account by adjusting the flow rate of the second gas 28 supplied to the reservoir 47. In addition, the second gas inlet 35 may be located upstream of the reservoir 47. In any event, as the second gas 28 enters the second passageway 22, a head of the second gas moves toward the patient interface 44 and residual gases downstream of the head can be discharged from the circuit 20 by the patient interface 44.

If the active valve mechanism 36 is operated to open the second valve 56 at the start of patient inhalation, residual gases in the third passageway 23 may delay the onset of the stored second gas 28 reaching the patient. The delay between the first gas 26 being supplied to the second passageway 33 by the second valve 56 opening and the stored second gas 28 reaching the patient is proportional to the volume of the third passageway 23. In the case of FIG. 13, the third passageway 23 will have a length of tubing that minimises weight or pull on the patient interface. As the internal volume of the third passageway 23 may be significant to a particular patient's tidal volume, it may be desirable to control the second valve 56 to compensate for this delay. To help take this into account, the breathing circuit 20 includes at least one sensor 49 that detects the breathing cycle of the patient 74, namely when the patient 74 is inhaling and exhaling.

The at least one sensor 49 may be located anywhere within the breathing circuit 20 or on the patient 74, as represented by the dashed line leading to the sensor 49 in FIG. 12. For example, the at least sensor 49 may be located on either, the patient interface 44, the first passageway 21, the second passageway 22, on the flow generator 33, or when multiple sensors 49 are provided, the sensors 49 may be provided on any combination of these. The at least one sensor 49 may measure flow rate, temperature, gas pressure, gas composition or any combination of these parameters to detect the breathing cycle of the patient. The controller 52 may receive an output signal of the sensor(s) 49, which the controller 52 uses to calculate, or has a process that calculates, the period of inhalation and/or exhalation, and in turn, produces a control output signal that is used to operate the active valve mechanism 36. Alternatively, the sensor(s) 49 may determine whether the patient is inhaling or exhaling and the controller 52 produces a control output signal that operates the active valve mechanism 36 to supply the first gas 26 or the second gas 28 depending on the output signal of the sensor(s) 49. In any event, the controller 52 may calculate the period of inhalation, and generate a control output signal to operate the active valve mechanism 36 that minimizes the delay between the active valve mechanism 36 supplying the first gas 26 to the second passageway 22 and the patient receiving the breathing gas from the second passageway 22. In other words, the internal volume of the third passageway 23 and the reservoir 47, can delay the onset of the second gas 28 being supplied to the patient, and the controller can reduce the delay by generating an output signal to operate the active valve mechanism 36 to pre-empt patient breathing. In addition, the flow generator 33 may also be controlled so that the flow of the first gas 26 conveyed along the second passageway 22 is reduced during patient inhalation, compared to during patient exhalation. This will take into consideration that the total flow of the breathing gas received by the patient interface 44 during inhalation is a function of the sum of the flow rates of the first gas 26 and the second gas 28 entering the second passageway 22 as described above in the relation to FIGS. 1 to 11.

FIG. 14 illustrates a breathing circuit 20 including first and second passageways 21 and 22 connected to a patient interface 44, and a flow assembly 24 including separate first and second flow generators 33A and 33B connected to the first and second flow passageways 21, 22 respectively. The flow assembly 24 also includes separate first and second humidification devices 50, 51 in the first and second passageways 21, 22 respectively, and a second gas source 27 connected to a distal portion 31 of the second passageway 22.

The first and second flow generators 33A and 33B can be operated during patient exhalation and patient inhalation respectively to supply the first gas 28 to the passageways 21, 22. Specifically, the first flow generator 33A can be operated during patient exhalation only, or in most instances, and the second flow generator 33B can be operated during patient inhalation only, or in most instances. It will also be appreciated that both the first flow generator 33A and the second flow generator 33B can both be operated to supply the first gas 28 to both the first and the second passageways 21 and 22 for an overlapping period, for example, when the breathing cycle changes from patient inhalation to patient exhalation or vice versa. One possible characteristic of the configuration shown in FIG. 14 is that the active valve mechanism 36 may not be required, as the first gas 26 is only conveyed by the first and second passageways 21, 22 when the respective flow generator 33A and 33B is operated. Although not illustrated, the two flow generators 33A and 33B may be combined in a single flow generator having, for example, first and second outlets that respectively connect to the first and second passageways 21, 22 and the first and second outlets are only opened during patient exhalation and inhalation respectively. In this configuration, an active valve mechanism described herein may be utilized to control flow from either the first outlet or the second outlet of the single flow generator.

With reference to FIG. 14, the breathing circuit 20 includes at least one sensor 49 that detects the breathing cycle of the patient 74, namely when the patient 74 is inhaling and exhaling. The at least one sensor 49 may be located anywhere within the breathing circuit 20 or on the patient 74, as represented by the dashed line leading to the sensor 49 in FIG. 14. For example, the at least sensor 49 may be located on either, the patient interface 44, the first passageway 21, the second passageway 22, on the flow generator 33, or when multiple sensors 49 are provided, the sensors 49 may be provided on any combination of these. The at least one sensor 49 may measure flow rate, temperature, gas pressure, gas composition or any combination of these parameters to detect the breathing cycle of the patient 74. The controller 52 may receive an output signal of the sensor(s) 49 which the controller 52 uses to calculate, or has a process that calculates, the period of inhalation and/or exhalation, and in turn, produces a control output signal that is used to operate the first and second flow generators 33A and 33B respectively. Alternatively, the sensor(s) 49 may determine whether the patient is inhaling or exhaling and the controller 52 produces a control output signal that operates the first and second flow generators 33A and 33B to supply the first gas 26 depending on the output signal of the sensor(s) 49. In addition, the second flow generator 33B may also be controlled so that the flow of the first gas 26 conveyed along the second passageway 22 is reduced during patient inhalation, compared to flow of the first gas 26 that is in the first passageway 21 that is controlled and generated by the first flow generator 33A during patient exhalation. This control of the first and second flow generators 33A and 33B can also take into consideration that the total flow of the breathing gas received by the patient interface 44 during inhalation is a function of the sum of the flow rates of the first gas 26 and the second gas 28 entering the second passageway 22 as described above in the relation to FIGS. 1 to 11.

The second gas inlet 35 is connected to the second passageway 22 upstream of the flow generator 33B and the humidification device 51. One possible characteristic with this configuration is that the internal volume for storing the second gas 28 will comprise the flow paths of the flow generator 33B and the second humidification device 51. This provides a greater volume for storing the second gas 28. Another characteristic of with this configuration is that the second gas 28 is humidified.

The breathing circuit 20 also has first and second non-return valves 37 and 38 on distal ends of the first and second passageways 21 and 22. The second non-return valve 38 provided upstream of the second gas inlet 35 may inhibit the second gas 28 from flowing upstream during patient exhalation. The first non-return valve 37 may inhibit flow along the first passageway 21 during patient inhalation, for instance flow from the second passageway 22 backwards up the first passageway 21, rather than going to the patient interface 44. The first non-return valve 37 could be located anywhere on first passageway 21, either on the distal or proximal portions 31 and 32.

In other examples the second gas inlet 35 may be located downstream of the second flow generator 33B or downstream of the humification device 46. Generally speaking, the second gas inlet 35 may be located in a distal portion 29 of the second passageway 22 as illustrated.

In another example, not illustrated in the FIG. 14, the second gas inlet 28 may be located in a proximal portion 29 of the second passageway 22. In this instance, a non-return valve may be located downstream of the second gas inlet 35 to inhibit the first gas 26 from passing from the first passageway 21 to the second passageway 22.

As an alternative to the first and second humidification devices 50, 51 shown, a single humidification device having dual chambers may be provided, in which one of each chamber forms part of the first and second passageways 21, 22.

The breathing circuit 20 shown in FIGS. 15 to 27 can be used for providing positive pressure respiratory therapy to a patient using a sealed patient interface. Examples include CPAP pressure therapy or bilevel pressure therapy which may be effective in increasing oxygenation of the patient's blood and/or reducing the work of breathing.

FIGS. 15 and 16 illustrate a breathing circuit 20 including first and second passageways 21, 22 in which distal portions 31, 29 respectively thereof are connected to a first gas source 25, and the distal portion 29 of the second passageway 22 is also connected to a second gas source 27. The breathing circuit 20 has a flow assembly 24 that includes: i) the first gas source 25, suitably a flow generator 33 that supplies filtered or unfiltered air as a first gas 26, ii) the second gas source 27, and iii) an active valve mechanism 36 that alternately opens and closes either the first passageway 21 or the second passageway 22 to a patient interface 44. It will also be appreciated that both the first and second valves 55 and 56 may be open for an overlapping period. For example, when the breathing cycle changes from patient inhalation to patient exhalation or vice versa, one of the valves 55 or 56 that is closed may move to an opened position for a period of time before the other valve 55 or 56 that is opened moves to a closed position.

The flow assembly 24 also includes a first gas inlet 34 at the distal portions 31, 29 of the first and second passageways 21, 22 that connects to an outlet of a flow generator 33 that supplies the first gas 26. The first gas inlet 34 may be any suitable three limb joiner such as a Y-shaped joiner or a T-shaped joiner. The three limb joiner acts as a splitter to supply the first gas 26 to the first and the second passageways 21, 22. Similarly, the second gas source 27 may be connected to the second passageway 22 at a second gas inlet 35 that may be any suitable three limb joiner.

The flow generator 33 may be operable to supply gas at a controlled pressure. For instance, the flow generator 33 may supply the first gas 26 between a first pressure during patient inhalation being an IPAP (inspiratory positive airway pressure) and a second pressure during patient exhalation being an EPAP (expiratory positive airway pressure). In another example, the flow generator 33 may supply the first gas at a constant pressure across the whole breathing cycle, e.g., CPAP.

The active valve mechanism 36 in FIGS. 15 and 16 includes two separate adjustable valves, namely a first valve 55 for the first passageway 21 that is opened in FIG. 15 and closed in FIG. 16, and a second valve 56 for the second passageway 22 that is closed in FIG. 15 and opened in FIG. 16. The opened/closed status of the first and second valves 55, 56 in FIG. 15 represents the first passageway 21 being in fluid communication with the patient interface 44 i.e., opened to the patient interface 44, and the second passageway 22 not being in fluid communication with the patient interface 44 i.e., is closed to the patient interface 44 and is the configuration of the flow assembly 24 and the passageways 21, 22 during patient exhalation. The opened/closed status of the first and second valves 55 and 56 in FIG. 16 represents the second gas passageway 22 being in fluid communication with the patient interface 44 i.e., opened to the patient interface 44, and the first passageway 21 not being in fluid communication with the patient interface 44, i.e., is closed to the patient interface 44, and is the configuration of the flow assembly 24 and the passageways 21, 22 during patient inhalation.

The first and second valves 55 and 56 shown in FIGS. 15 and 16 may be any suitable two port valve having an inlet and an outlet. The first and second valves 55 and 56 may be an actively-controlled valve 36. The actively-controlled valve 36 may be, for example, adjusted by any suitable actuator 69, such as a solenoid actuator, a pneumatic actuator, an electric motor and so forth. Examples of suitable valves include shuttle valves, spool valves, ball valves, gate valves, butterfly valves, a diaphragm valve switch valves and so forth.

Although not illustrated, the first and second valves 55 and 56 may be substituted with a single three-way valve, such as a shuttle valve or a spool valve having one outlet connected to the patient interface 44 and two inlets, one of each being connected to the first and second passageways 21 and 22 respectively. The three-way valve may be an actively-controlled valve 36. The valve may be driven by suitable actuator 69, such as a solenoid actuator, a pneumatic actuator, an electric motor and so forth.

During patient exhalation the first gas 26 is supplied simultaneously to the distal portions 31 and 29 of the first and second passageways 21 and 22, as shown in FIG. 15, and the active valve mechanism 36 is configured to allow supply of the first gas 26 to the patient interface 44 via the first passageway 21 and disallow supply of the first gas 26 to the patient interface 44 via the second passageway 22. The first passageway 21 includes an exhalation port 70, located in a proximal portion of the first passageway 21 that vents exhaled gas of the patient during exhalation and may prevent overpressure of the beathing circuit 20. As shown in FIGS. 15 and 16, the exhalation port 70 may be located downstream of the first valve 55. In another example, not illustrated, the exhalation port 70 may be located upstream of the first valve 55 in a proximal portion of the first passageway 21. In addition to the exhaled gas being vented by the exhalation port 70 from the circuit 20, other gases, including the first gas 26 supplied by the flow generator 33, can be vented by the exhalation port 70 to maintain the required therapeutic pressure during exhalation. Other gases that can also be vented include residual breathing gases that were not inhaled, such as gases in the dead space of the patient interface 44, and tubing connected to the patient interface 44. The flow generator 33 will generally provide relatively small flows to ensure the correct therapeutic pressure is maintained. The exhalation port 70 may be any suitable flow restriction including a restriction orifice, a control valve, a positive end expiratory pressure valve (PEEP valve), an aperture of fixed size, or a controlled valve outlet. One of the benefits in having the exhalation port 70 located in the proximal portion of the first passageway 21 is that there is a low risk of the breathing gas from the second passageway 22 being vented from the circuit 20 during the inhalation.

Although not shown, the circuit 20 may have a filter, for example at an air inlet of the flow generator 33 for supplying the first gas 26 as filtered air via the first passageway 21 to the patient interface 44. One or more filters may also be located in the first and second passageways 21 and 22 between the first and second valves 55 and 56 and the patient interface 44.

Although not illustrated in FIGS. 15 and 16, the circuit 20 may have a heat and moisture exchanger (HME). The HME may be in addition to, or as an alternative to a humidifier (not illustrated). The HME may be located at or near the patient interface 44, for example in passageway 23. The HME may humidify and heat the breathing gases provided to the patient interface 44. The HME may be configured to allow for bidirectional flow. In configurations with an HME, the exhalation port 70 may be located on the side of the HME opposite the patient, for example on the first passageway 21 or on the passageway 23.

In addition, the flow assembly 24 includes a vent 45 extending from the second passageway 22 upstream of the second valve 56. During patient exhalation, the second gas 28 enters the second passageway 22 and is stored in the second passageway 22. The second gas 28 can be supplied into the second passageway 22 at a higher pressure (for example a slightly higher pressure) than the first gas 26, inhibiting the first gas 26 from flowing downstream of the second gas inlet 35 during patient exhalation and ensuring the second gas accumulates in the second passageway 22. Residual breathing gas downstream of a head of the second gas 28 flowing in a direction toward the patient can be vented by the vent 45. In addition, the vent 45 allows the second gas 28 to flow into the second passageway 22 during patient exhalation without increasing the pressure in the second passageway 22 or flowing upstream and entering the first gas passageway 21. The vent 45 may be arranged as any suitable flow restriction including a restriction orifice, a control valve, a positive end expiratory pressure valve (PEEP valve), an aperture of fixed size, or a controlled valve outlet. In one example, the vent 45 is arranged to discharge gas from the second passageway 22 at a rate at which the second gas 28 enters the second passageway 22. If required, a control valve, not illustrated, can be used for regulating the flow of the second gas 28 into the second passageway 22. Similarly, a vent sensor 75 can be used to measure the rate at which gas is discharged from the vent 45, and an output of the flow sensor 75 can be used to operate the control valve 76 to regulate the rate at which the second gas 28 enters the second passageway 22. In one example, the vent 45 may comprise a valve that can close the vent 45 during patient inhalation. That is, the vent 45 may be closed when the second valve 56 is open. This ensures that during patient inhalation, second gas that has accumulated in the second passageway is not exhausted from the vent 45, and/or ambient air is not drawn into the second passageway through vent 45.

Although not shown in FIGS. 15 and 16, the flow assembly 24 may include a non-return valve upstream of the second gas inlet 35, such as between the first gas inlet 34 and the second gas inlet 35 for inhibiting the second gas 28 flowing into the first passageway 21.

At the end of patient exhalation, or at a pause between patient exhalation and patient inhalation, the active valve mechanism 36 switches from the configuration shown in FIG. 15 to the configuration shown in FIG. 16. That is to say, the first gas 26 simultaneously supplied to the first and second passageway 21 and 22 is allowed to flow along the second passageway 22 by the second valve 56 being opened, and is inhibited from flowing along the first passageway 21 by the first valve 55 being closed. At least during the initial stages of patient inhalation, the patient receives the second gas 28 that has been stored in the second passageway 22, such as oxygen gas to provide a therapeutic benefit. Specifically, the second gas 28 can comprise high concentrations of the oxygen gas can be drawn into the alveoli of the patient's lungs which in turn, can increase the oxygen levels in the patient's blood. Throughout patient inhalation, the first gas 26 is supplied to the distal portion 29 of the second passageway 22 so that when the second gas 28 stored in the second passageway 22 has been supplied to the patient interface 44 and the patient continues to inhale, the patient 74 will receive a mixture of the first gas 26 and the second gas 28 until inhalation has finished. The second gas 28 may enter the second passageway 22 at a constant rate throughout the breathing cycle of the patient 74.

The active valve mechanism 36, and indeed the first and second valves 55 and 56 can change between opened and closed positions in response to outputs of at least one sensor 49 that detects the breathing cycle of the patient 74. The at least one sensor 49 may be located anywhere within the breathing circuit 20 or on the patient 74, as represented by the dashed line leading to the sensor 49 in FIG. 1. For example, the at least one sensor 49 may be located on either, the patient interface 44, the first passageway 21, the second passageway 22, on the flow generator 33, or when multiple sensors 49 are provided, the sensors 49 may be provided on any combination of these. The at least one sensor 49 may measure flow rate, temperature, gas pressure, gas composition or any combination of these parameters (or other parameters) to detect the breathing cycle of the patient 74. A controller 52 may receive an output of the sensor 49 in order to control operation of either the flow generator, or the active valve mechanism, or a combination of both. The controller 52 may also operate the active valve mechanism 36 to open and close the first and second valves 55 and 56. Further details of the sensor 49 and, optional control for operating the active valve mechanism 36 is described in detail with reference to FIG. 44 below.

The sensor 49 and the controller 52 can operate the flow generator 33 to provide the required level of pressure therapy. For example, in the range of 4 to 40 cmH₂O. In addition, the specification of International patent publication number WO2010021556A1 (PCT/NZ2009/000172), entitled “Breathing transition detection”, filed 19 Aug. 2009, describes breathing detection methods from sensors that could be implemented with the embodiments described herein.

The controller 52 can operate the active valve mechanism 36 independently of the operation of the flow generator 33. The sensor 49 and controller 52 can also operate the flow generator 33 independently of the flow rate of the second gas 28 that accumulates in the second passageway 22. For instance, a flow sensor (not illustrated) on the vent 45 and a control valve (not illustrated) on the second gas inlet 35 for controlling the flow of the second gas 28 into the second passageway 22, can both be operated independently of operation of the flow generator 33.

The breathing circuit 20 illustrated in FIGS. 17 and 18 is the same as the breathing circuit 20 shown in FIGS. 15 and 16 save for the at least one sensor 49 and the controller 52 have been omitted from FIGS. 17 and 18 and the first and second valves 55 and 56 are both in the opened position. In addition, FIGS. 17 and 18 also illustrate two further locations of the exhalation port 70. In the case of FIG. 17, the exhalation port 70 is located on the patient interface 44. In this instance, the exhalation port 70 may include bias holes in the interface and/or a dedicated exhalation port located on the patient interface 44. A benefit in having the exhalation port in this location is that it minimizes the dead space in the circuit where CO₂ and/or other exhaled gases may accumulate and be rebreathed. In other words, there is minimal volume between the patient and the exhalation port 70. In FIG. 18 the exhalation port 70 is located in the distal portion 31 of the first passageway 21. A benefit in having the exhalation port 70 in this location is that it reduces the likelihood of the second gas 28 leaking out the exhalation port 70 without being inhaled. This is because the supply path for the second gas 28 from the second passageway to the patient 72 is spaced from the exhalation port 70 by the length of the first passageway 21. The exhalation port 70 may also prevent overpressure of the breath circuit 20.

FIGS. 19 and 20 illustrate a breathing circuit 20 similar to the breathing circuit 20 shown in FIGS. 15 and 16, in which distal portions 31 and 29 of first and second passageways 21 and 22 are connected to a first gas source 25, suitably a flow generator 33 that blows filtered or unfiltered air as a first gas 26, and an active valve mechanism 36 that alternately opens and closes either the first passageway 21 or the second passageway 22 to a patient interface 44. In one example, the flow generator 33 can supply the first gas 26 at a controlled pressure. For instance, the flow generator 33 may supply the first gas 26 between a first pressure during patient inhalation being an IPAP (inspiratory positive airway pressure) and a second pressure during patient exhalation being an EPAP (expiratory positive airway pressure). In another example, the flow generator 33 may supply the first gas at a constant pressure across the whole breathing cycle, e.g., CPAP. The description of these features in FIGS. 15 and 16 applies equally to the breathing circuit 20 of the FIGS. 19 and 20.

The second gas inlet 35 in FIGS. 19 and 20 is located in a distal portion 29 of the second passageway 22. During patient exhalation, the first and second passageways 21, 22 and the flow assembly 24 have a configuration as shown in FIG. 19. Specifically, the second valve 56 is closed which inhibits fluid communication from the second passage 22 to the patient interface 44, and the first valve 55 is opened which provides fluid communication from the first passageway 21 to the patient interface 44 and allows the first gas 26 to be supplied to the patient interface 44. In addition, during exhalation, the second gas 28 enters the second passageway 22 and flows toward the patient interface 44, that is to say in an downstream direction relative to the direction of flow during patient inhalation. The second gas 28 entering the second passageway 22 during patient exhalation is stored therein. A volume of the second gas 28 can be stored in the second passageway 22 which may include the second humidification device 51. The second gas 28 may enter at a higher pressure than the pressure at which the first gas 26 enters. The breathing circuit 20 also has a vent 45 for venting residual gas from the second passageway 22 when the second valve 56 is closed and as the head of second gas 28 flows from the second inlet 35 toward the second valve 56. In addition, although not illustrated, the circuit 20 may also include a non-return valve in the second passageway 22, for example located between the second gas inlet 35 and the first gas inlet 34, that is arranged to inhibit flow of the second gas 28 from the second passageway 22 to the first passageway 21. In addition, the circuit 20 has an exhalation port 70 on the first passageway 21 that vents exhaled gas, residual gas from dead space in the circuit 20 between the interface and the exhalation port 70. In the event that the exhaling action of the patient meets the required therapeutic pressure, the flow generator 33 can be operated to provide no or reverse flow. If required, the exhalation port 70 can also vent the breathing circuit 20 to prevent overpressure of the beathing circuit 20. One of the benefits in having the exhalation port 70 located in the proximal portion of the first passageway 21 is that there is a low risk of the breathing gas from the second passageway 22 being vented from the circuit 20 during the inhalation.

During patient inhalation, the first and second passageways 21, 22 and the active valve assembly 36 have a configuration as shown in FIG. 20. Specifically, first valve 55 is closed which inhibits fluid communication from the first passage 21 to the patient interface 44, and the second valve 56 is opened which provides fluid communication from the second passageway 22 to patient interface 44 and allows the first gas 26 to flow along the first passageway 21. The first gas 26 flowing along the second passageway 22 displaces the second gas 28 stored in the second passageway 22 so as to supply the second gas 28 to the patient interface 44. If all of the stored second gas 28 in the second passageway 22 has been inhaled and patient inhalation continues, a mixture of the second gas 28 and first gas 26 can be supplied to the patient interface 44.

The active valve mechanism 36, and indeed the first and second valves 55 and 56 can change between opened and closed positions in response to outputs of at least one sensor 49 that detects the breathing cycle of the patient 74. In most instances, either the first valve 55 or the second valve 56 is opened and the other is closed. However, it will also be appreciated that both the first and second valves 55 and 56 may be open for an overlapping period, for example, when the breathing cycle changes from patient inhalation to patient exhalation or vice versa. The at least one sensor 49 may be located anywhere within the breathing circuit or on the patient 74, as represented by the dashed line leading to the sensor 49 in FIGS. 19 and 20. For example, the at least sensor 49 may be located on either, the patient interface 44, the first passageway 21, the second passageway 22, on the flow generator 33, or when multiple sensors 49 are provided, the sensors may be provided on any combination of these. In addition to gas flow rate, the sensor 49 may measure temperature, gas pressure or gas composition to detect the breathing cycle 20 of the patient 74. A controller 52 may receive an output of the sensor 49 in order to control operation of either the flow generator 33, or the active valve mechanism 36, or a combination of both. The controller 52 may also operate the active valve mechanism 36 to open and close the first and second valves 55 and 56. Further details of the sensor 49 and, optional control for operating the active valve mechanism 36 is described in detail with reference to FIG. 44 below.

The sensor 49 and the controller 52 can operate the flow generator 33 to provide the required level of pressure therapy By way of example, the pressure therapy may provide pressure from 4 to 40 cmH₂O. In addition, the specification of International patent publication number WO2010021556A1 (PCT/NZ2009/000172), entitled “Breathing transition detection”, filed 19 Aug. 2009, describes breathing detection methods from sensors that could be implemented with the embodiments described herein.

The controller 52 can operate the active valve mechanism 36 independently of the operation of the flow generator 33. The sensor 49 and controller 52 can also operate the flow generator 33 independently of the flow rate of the second gas 28 that accumulates in the second passageway 22. For instance, a vent sensor 75 can be used to measure the rate at which gas is discharged from the vent 45, and an output of the flow sensor 75 can be used to operate the control valve 76 to regulate the rate at which the second gas 28 enters the second passageway 22

FIGS. 19 and 20 also illustrate the flow assembly 24 including dedicated first and second humidification devices 50, 51 in the first and second passageways 21, 22 respectively. The first humidification device 50 humidifies the first gas 26 flowing along the first passageway 21. The second humidification device 51 is located at a distal portion 29 of the second passageway 22 and the second gas inlet 35 so that a head of the second gas 28 enters the second humidification device 51 during patient exhalation and depending on the volume of the second gas 28 to be stored, the head of the second gas 28 can continue toward the active valve mechanism 36. The second humidification device 51 therefore provides a greater volume for storing the second gas 28. In this situation, the second humidification device 51 may be located as close as possible to the second gas inlet 35.

Alternatively, the second humidification device 51 may be located in the proximal portion 30 and the head of the second gas 28 may be intended to not enter the second humidification device 51.

If required, either one or both of the first and second humidification devices 50 and 51 illustrated in FIGS. 19 and 20 can be omitted. In addition, in the event that second gas 28 is required to be humidified, a humidification device (not shown) can be located between the source of the second gas 28 and the second gas inlet 35.

Although not illustrated in FIGS. 19 and 20, the circuit 20 may have a heat and moisture exchanger (HME). The HME may be in addition to, or as an alternative to a humidifier 50, 51. The HME may be located at or near the patient interface 44, for example in passageway 23. The HME may humidify and heat the breathing gases provided to the patient interface 44. The HME may be configured to allow for bidirectional flow. In configurations with an HME, the exhalation port 70 may be located on the side of the HME opposite the patient, for example on the first passageway 21 or on the passageway 23.

The breathing circuit 20 illustrated in FIGS. 21 and 22 is the same as the breathing circuit 20 shown in FIGS. 19 and 20 save for the at least one sensor 49 and the controller 52 have been omitted from FIGS. 21 and 22 and the first and second valves 55 and 56 are both in the opened position. As explained above, during operation only one of the first or second valves 55 and 56 will be opened at any one time. In addition, FIGS. 21 and 22 also illustrate two further locations of the exhalation port 70. In the case of FIG. 21, the exhalation port 70 is located on the patient interface 44. In this instance, the exhalation port 70 may include bias holes in the interface and/or a dedicated exhalation port 70 located on the patient interface 44. A benefit in having the exhalation port 70 in this location is that it minimizes the dead space in the circuit where CO₂ and/or other exhaled gases may accumulate and be rebreathed. In FIG. 22 the exhalation port 70 is located in the distal portion 31 of the first passageway 21 Which can reduce the likelihood of the second gas 28 leaking out the exhalation port 70 without being inhaled.

In FIG. 22, the active valve mechanism 36 is located in the proximal portions 32 and 30 of the first and second passageways 21 and 22 respectively, and the second gas inlet 35 is located in the distal portion 29 of the second passageway 22. In this situation, the venting valve 45 may be located between the active valve mechanism 36 and the second gas inlet 35, and suitably adjacent to the active valve mechanism 36.

FIGS. 23 and 24 illustrate a breathing circuit 20 including first and second passageways 21, 22 both having proximal portions 32 and 30 connected to a patient interface 44 and a flow assembly 24 having an active valve mechanism 36. The active valve mechanism 36 is located in proximal portions 32 and 30 of the first passageway 21 and the second passageway 22. A flow generator 33 is connected to distal portion 29 and 31 of the first and second passageways 21 and 22 for conveying filtered or unfiltered air as a first gas 26 to the patient interface 44.

The active valve mechanism 36 in FIGS. 23 and 24 includes two separate adjustable valves, namely a first valve 55 for the first passageway 21 that is opened in FIG. 23 and closed in FIG. 24, and a second valve 56 for the second passageway 22 that is closed in FIG. 23 and opened in FIG. 24. Specifically, the opened/closed status of the first and second valves 55 and 56 in FIG. 23 represents the first passageway 21 being in fluid communication with the patient interface 44, and the second passageway 22 not being in fluid communication with the patient interface 44. Similarly, the opened/closed status of the first and second valves 55 and 56 in FIG. 24 represents the second passageway 22 being in fluid communication with the patient interface 44, and the first passageway 21 not being in fluid communication with the patient interface 44. In addition, it will also be appreciated that both the first and second valves 55 and 56 may be open for an overlapping period, for example, when the breathing cycle changes from patient inhalation to patient exhalation or vice versa, one of the valves 55 or 56 that is closed may move to an opened position for a period of time before the other valve 55 or 56 that is opened moves to a closed position.

The active valve mechanism 36 may include an actuator 69 for operating the active valve mechanism 36 by adjusting the first and second valves 55, 56. The actuator 69 may be any suitable actuator, such as a solenoid actuator, a pneumatic actuator, an electric motor and so forth. Examples of suitable valves include shuttle valves, spool valves, ball valves, gate valves, butterfly valves, a diaphragm valve, a switch valve and so forth. In another example, the active valve mechanism 36 may also include a single three-way valve, such as a shuttle valve or a spool valve having one inlet for connection to the output of the flow generator 33 and two outlets, one of each outlet being connected to the first and second passageways 21 and 22 respectively.

Referring to FIG. 23, during patient exhalation, the first valve 55 is opened and the first gas 26 flows from the first gas source 25 along the first passageway 21 to supply the first gas 26 to the patient interface 44. It will also be appreciated that both the first and second valves 55 and 56 may also be open for an overlapping period when the beathing cycle is change for inhalation to exhalation and vice versa. In any event, when the second valve 56 is closed and the second gas 28 enters the second passageway 22 at the second gas inlet 35 located in the proximal portion 30 of the second passageway 22, a head of the second gas 28 flows in a direction away from the patient interface 44 to store a volume of the second gas 28 in the second passageway 22. The exhaled gas is vented from the circuit via exhalation port 70 located in the proximal portion of the first passageway 21. In addition, residual gases in the second passageway 22, such as gases upstream of the second gas 28 accumulating in the second passageway 22 can be displaced toward the first passageway 21. Overpressure of the breathing circuit 20 can be prevented by venting from the exhalation port 70. The second gas 28 is supplied at a higher pressure (for example a slightly higher pressure) than the first gas 26 so that the first gas 26 is inhibited from passing from the first passageway 21 to the distal portion 29 of the second passageway 22. One of the characteristics in having the exhalation port 70 located in the proximal portion of the first passageway 21 is that there is a low risk of the breathing gas from the second passageway 22 being vented from the circuit 20 during the inhalation.

Referring to FIG. 24, during patient inhalation the second valve 56 is opened, and the first gas 26 flows form the first gas source 25 to and along the second passageway 22 to supply the first gas 26 upstream of the second gas inlet 35. At the start of patient inhalation, the first gas 26 flowing along the second passageway 22 displaces the second gas 28 stored in the second passageway 22 to supply the second gas to the patient interface 44. If all of the stored second gas 28 in the passageway has been inhaled and patient inhalation continues, a mixture of the second gas 28 and first gas 26 can be supplied to the patient interface 44. The second gas 28 can enter the second passageway 22 at a constant rate throughout the breathing cycle. However, if required, the flow rate of the second gas 28 can be controlled, for example, by a control valve to adjust the flow of the second gas 28 through the second gas inlet 35

As can be seen, the flow assembly 24 also includes dedicated first and second humidification devices 50, 51 in the first and second passageways 21, 22. The first humidification device 50 humidifies the first gas 26 conveyed by the first passageway 21. The second humidification device 51 is located upstream of the second gas inlet 35 and can humidify any gases therein, including the first gas 26 conveyed through the humidification device 51 during patient inhalation, and any of the second gas 28 that is stored in the humidification device 51 during patient exhalation.

If required, either one of the first and second humidification devices 50 and 51 may be omitted or removable from the breathing circuit 20.

The active valve mechanism 36, and indeed the first and second valves 55 and 56 can change between opened and closed positions in response to outputs of at least one sensor 49 that detects the breathing cycle of the patient. The at least one sensor 49 may be located anywhere within the breathing circuit 20 or on the patient, as represented by the dashed line leading to the sensor 49 in FIGS. 23 and 24. For example, the at least sensor 49 may be located on either, the patient interface 44, the first passageway 21, the second passageway 22, on the flow generator 33, or when multiple sensors 49 are provided, the sensors 49 may be provided on any combination of these. In addition to gas flow rate, the sensor 49 may measure temperature, gas pressure or gas composition to detect the breathing cycle of the patient 74. A controller 52 may receive an output of the sensor 49 which is used to control operation of either the flow generator 33, or the active valve mechanism 36, or a combination of both. The controller 52 may also operate the active valve mechanism 36 to open and close the first and second valves 55 and 56. Further details of the sensor 49 and, optional control for operating the active valve mechanism 36 is described in detail with reference to FIG. 44 below.

The sensor 49 and the controller 52 can operate the flow generator 33 to provide the required level of pressure therapy. By way of example, the pressure therapy may provide pressure from 4 to 40 cmH₂O. In addition, the specification of International patent publication number WO2010021556A1 (PCT/NZ2009/000172), entitled “Breathing transition detection”, filed 19 Aug. 2009, describes breathing detection methods from sensors that could be implemented with the embodiments described herein.

The controller 52 can operate the active valve mechanism 36 independently of the operation of the flow generator 33. The sensor 49 and controller 52 can also operate the flow generator 33 independently of the flow rate of the second gas 28 that accumulates in the second passageway 22. For instance, a flow sensor (not illustrated) on the vent 45 and a control valve (not illustrated) on the second gas inlet 35 for controlling the flow of the second gas 28 into the second passageway 22, can both be operated independently of operation of the flow generator 33.

Although not illustrated in FIGS. 23 and 24, the circuit 20 may have a heat and moisture exchanger (HME). The HME may be in addition to, or as an alternative to a humidifier 50, 51. The HME may be located at or near the patient interface 44, for example in passageway 23. The HME may humidify and heat the breathing gases provided to the patient interface 44. The HME may be configured to allow for bidirectional flow. In configurations with an HME, the exhalation port 70 may be located on the side of the HME opposite the patient, for example on the first passageway 21 or on the passageway 23.

The breathing circuit 20 illustrated in FIGS. 25 and 26 is the same as the breathing circuit 20 in FIGS. 23 and 24 save for the at least one sensor 49 and the controller 52 have been omitted from FIGS. 25 and 26 and the first and second valves 55 and 56 are both in the opened position. As explained above, during operation in most instances only one of the first or second valves 55 and 56 will be opened at any one time, and the circuit may include sensor(s) 49 and the controller 52. However, it will also be appreciated that both the first and second valves 55 and 56 may be open for an overlapping period, for example, when the breathing cycle changes from patient inhalation to patient exhalation or vice versa. In addition, FIGS. 25 and 26 also illustrate two further locations of the exhalation port 70. In the case of FIG. 25, the exhalation port 70 is located on the patient interface 44. In this instance, the exhalation port 70 may include bias holes in the interface and/or a dedicated exhalation port located on the patient interface 44. A benefit in having the exhalation port 70 in this location is that it minimises the dead space in the circuit where CO₂ and/or other exhaled gases may accumulate and be rebreathed. In FIG. 26 the exhalation port 70 is located in the distal portion 31 of the first passageway 21 which can reduce the likelihood of the second gas 28 leaking out the exhalation port without being inhaled.

FIGS. 27 and 28 illustrate a breathing circuit 20 including first and second passageways 21, 22 both having proximal portions 32 and 30 connected to a patient interface 44, and a flow assembly 24 having an active valve mechanism 36 in which distal portions 31 and 29 of the first passageway 21 and the second passageway 22 are alternately opened and closed to a first gas source 25, suitably in the form of a flow generator 33 blowing filtered or unfiltered air as a first gas 26. The opened/closed status of the first and second valves 55 and 56 in FIG. 27 represents the first passageway 21 being in fluid communication with the first gas source 25, and the second passageway 22 not being in fluid communication with the first gas source 25. Similarly, the opened/closed status of the first and second valves 55 and 56 in FIG. 28 represents the second passageway 22 being in fluid communication with first gas source 25, and the first passageway 21 not being in fluid communication with the first gas source 25. It will also be appreciated that both the first and second valves 55 and 56 may be open for an overlapping period, for example, when the breathing cycle changes from patient inhalation to patient exhalation or vice versa. The active valve mechanism 36 in FIGS. 27 and 28 includes two separate adjustable valves, namely a first valve 55 for the first passageway 21 that is opened in FIG. 27 and closed in FIG. 28, and a second valve 56 for the second passageway 22 that is closed in FIG. 27 and opened in FIG. 28.

The active valve mechanism 36 may be an actively-controlled valve. The actively-controlled valve may be adjust be, for example, any suitable actuator 69, such as a solenoid actuator, a pneumatic actuator, an electric motor and so forth. Examples of suitable valves include shuttle valves, spool valves, ball valves, gate valves, butterfly valves, a diaphragm valve switch valves and so forth. In another example, the active valve mechanism 36 may also be a single three-way valve, such as a shuttle valve or a spool valve having one inlet for connection to the output of the flow generator 33 and two outlets, one of each outlet being connected to the first and second passageways 21 and 22 respectively.

Referring to FIG. 27, during patient exhalation, the first valve 55 is opened and the first gas 26 flows from the first gas source 25 along the first passageway 21 to supply the first gas 26 to the patient interface 44. In addition, the second valve 56 is closed and the second gas 28 enters the second gas passageway 22 at the second gas inlet 35 located in the distal portion 29 so that a head of the second gas 28 flows in a direction toward the patient interface 44 to store a volume of the second gas 28 in the second passageway 22. As can be seen the second gas inlet 35 is located between the second humidifier 51 and the second valve 56. Residual gases in the second passageway 22 downstream of the head of the second gas 28 are also supplied to the patient interface 44

The first passageway 21 includes an exhalation port 70, located in a proximal portion of the first passageway 21 that vents exhaled gas of the patient during exhalation and may prevent overpressure of the beathing circuit 20. As shown in FIGS. 27 and 28, the exhalation port 70 may be located downstream of the first valve 55. In addition to the exhaled gas being vented by the exhalation port 70 from the circuit 20, other gases, including the first gas 26 supplied by the flow generator, can be vented by the exhalation port 70 to maintain the required therapeutic pressure during exhalation (and during inhalation). Other gases that can also be vented include residual breathing gases that were not inhaled, such as gases in the dead space of the patient interface 44, and tubing connected to the patient interface 44. When the patient is exhaling, much of the pressure for positive pressure therapy is provided by the exhaling action of the patient 74. As such, the flow generator 33 will generally provide relatively small flows to ensure the correct therapeutic pressure is maintained. This means that in some situations, the exhalation port 70 may not be needed to vent the first gas 26 from the flow generator 33 at some points during exhalation, typically during peak expiratory flow. The exhalation port 70 may be any suitable flow restriction including a restriction orifice, a control valve, a positive end expiratory pressure valve (PEEP valve), an aperture of fixed size, or a controlled valve outlet. One of the benefits in having the exhalation port 70 located in the proximal portion of the first passageway 21 is that there is a low risk of the breathing gas from the second passageway 22 being vented from the circuit 20 during the inhalation.

In addition, the second gas 28 is supplied at a higher pressure (for example a slightly higher pressure) than the first gas 26 so that second gas 28 continues to enter the second passageway 22 during patent inhalation and patient exhalation, and the exhaled gas and any of the first gas 26 that is not inhaled is vented from the exhalation port 70. If required, a non-return valve, not illustrated, can be provided in the second passageway 22 to inhibit flow in a direction from a proximal portion 30 toward a distal portion 29

Referring to FIG. 28, during patient inhalation the second valve 56 is opened, and the first gas 26 flows form the first gas source 25 to and along the second passageway 22 to supply the first gas 26 upstream of the second gas inlet 35. At the start of patient inhalation, the first gas 26 flowing along the second passageway 22 displaces the second gas 28 stored in the second passageway 22 to supply the second gas to the patient interface 44. If all of the stored second gas 28 in the passageway has been inhaled and patient continues to inhale, a mixture of the second gas 28 and first gas 26 can be supplied to the patient interface 44. The second gas 28 can enter the second passageway 22 at a constant rate throughout the breathing cycle. However, if required, the flow rate of the second gas 28 can be controlled, for example, by a control valve to adjust the flow of the second gas 28 through the second gas inlet 35.

With reference to FIG. 27, the breathing circuit 20 includes at least one sensor 49 that detects the breathing cycle of the patient 74, namely when the patient 74 is inhaling and exhaling. The at least one sensor 49 may be located anywhere within the breathing circuit 20 or on the patient 74, as represented by the dashed line leading to the sensor 49 in FIG. 27. For example, the at least sensor 49 may be located on either, the patient interface 44, the first passageway 21, the second passageway 22, on the flow generator 33, or when multiple sensors 49 are provided, the sensors 49 may be provided on any combination of these. In addition to gas flow rate, the sensor 49 may measure temperature, gas pressure or gas composition to detect the breathing cycle of the patient 74. A controller 52 may receive an output of the sensor 49 to control operation of either the flow generator, or the active valve mechanism, or a combination of both.

The sensor 49 and the controller 52 can operate the flow generator 33 to provide the required level of flow therapy. By way of example, the specification of International patent publication number WO2010021556A1 (PCT/NZ2009/000172), entitled “Breathing transition detection”, filed 19 Aug. 2009, describes breathing detection methods from sensors that could be implemented with the embodiments described herein. In any event, the controller 52 can operate the active valve mechanism 36 independently of the operation of the flow generator 33. The sensor 49 and controller 52 can also operate the flow generator 33 independently of the flow rate of the second gas 28 that accumulates in the second passageway 22.

As can be seen, the flow assembly 24 also includes dedicated first and second humidification devices 50, 51 in the first and second passageways 21, 22. The first humidification device 50 humidifies the first gas 26 conveyed by the first passageway 21. The second humidification device 51 is located downstream of the second gas inlet 35 and humidifies the second gas 28 entering during patient exhalation, and humidifies the first gas 26 and the second gas 28 flowing along the second gas passageway 22 during patient inhalation.

If required, either one of the first and second humidification devices 50 and 51 may be omitted or removable from the breathing circuit 20.

Although not illustrated in FIGS. 27 and 28, the second gas inlet 35 may be located at a proximal portion 30 of the second gas passageway 22. In this instance, a vent 45 may be used to discharge any residual gas in the second passageway 22 as the second gas 28 is stored therein during patient exhalation. The vent 45 may be located at a distal portion of the second passageway 22. In addition, a valve such as non-return valve or an actively controlled valve may be located between the second gat inlet 35 and the patient interface 44.

Although not illustrated in FIGS. 27 and 28, the circuit 20 may have a heat and moisture exchanger (HME). The HME may be in addition to, or as an alternative to a humidifier 50, 51. The HME may be located at or near the patient interface 44, for example in passageway 23. The HME may humidify and heat the breathing gases provided to the patient interface 44. The HME may be configured to allow for bidirectional flow. In configurations with an HME, the exhalation port 70 may be located on the side of the HME opposite the patient, for example on the first passageway 21 or on the passageway 23.

The breathing circuit 20 illustrated in FIGS. 29 and 30 is the same as the breathing circuit 20 shown in FIGS. 27 and 28, although the at least one sensor 49 and the controller 52 have been omitted from FIGS. 29 and 30 to simplify these Figures, and the first and second valves 55 and 56 are both in the opened position. As explained above, during operation in most instances only one of the first or second valves 55 and 56 will be opened at any one time. It will also be appreciated that both the first and second valves 55 and 56 may be open for an overlapping period, for example, when the breathing cycle changes from patient inhalation to patient exhalation or vice versa. In addition, FIGS. 29 and 30 also illustrate two further locations of the exhalation port 70. In the case of FIG. 29, the exhalation port 70 is located on the patient interface 44. In this instance, the exhalation port 70 may include bias holes in the interface 44 and/or a dedicated exhalation port 70 located on the patient interface 44. A benefit in having the exhalation port 70 in this location is that it minimises the dead space in the circuit 20 where CO₂ and/or other exhaled gases may accumulate and be rebreathed. In FIG. 30 the exhalation port 70 is located in the distal portion 31 of the first passageway 21 which can reduce the likelihood of the second gas 28 leaking out the exhalation port 70 without being inhaled.

FIGS. 31 and 32 illustrate a breathing circuit 20 that is the same as the breathing circuit 20 shown in FIGS. 27 and 28, save for the first and second humidification devices 50 and 51 being replaced with a single humidification device 46 located downstream of the first gas source 25 and upstream of the first and second passageways 21 and 22. Suitably the humidification device 46 is located between the outlet of the first gas source 25 and upstream of the active valve mechanism 36. In this circuit 20, the first gas 26 supplied to the patient interface 44 will be humidified, whereas the second gas 28 will not be separately humidified. If required, a further humidification device may be provided to humidify the second gas 28 prior to being supplied to the second gas inlet 35. Although not illustrated, it is also possible that the flow generator 33 and the humidification device 46 may be integrated together, or connected together so as to effectively form a single equipment item.

The second gas 28 can be supplied to the second passageway 22 at a higher pressure than the first gas 26, as such the first gas 26 is unlikely to flow from the proximal portion 32 of the first passageway 21 to the proximal portion 30 of the second passageway 22. In addition, a non-return valve, not illustrated, may be located in a proximal portion 30 of the second passageway 22 to inhibit the first gas 26 from passing from the first passageway 21 to the second passageway 22 during patient exhalation.

To facilitate use with a sealed patient interface 44, the flow assembly includes an exhalation port 70 for venting exhaled gas from the circuit 20. Depending on the level of positive pressure therapy provided, the first gas 26 supplied by the flow generator 33 and the residual gas downstream of the head of the second gas 28 can be vented from circuit via the exhalation port 70. That is to say, the exhalation port 70 can also prevent overpressure of the beathing circuit 20.

The breathing circuit 20 illustrated in FIGS. 33 and 34 is the same as the breathing circuit 20 shown in FIGS. 31 and 32 save for the at least one sensor 49 and the controller 52 have been omitted from FIGS. 31 and 32 to simplify the Figures, and the first and second valves 55 and 56 are both in the opened position. As explained above, during operation in most instances only one of the first or second valves 55 and 56 will be opened at any one time. However, it will also be appreciated that both the first and second valves 55 and 56 may be open for an overlapping period, for example, when the breathing cycle changes from patient inhalation to patient exhalation or vice versa. In addition, FIGS. 33 and 34 also illustrate two further locations of the exhalation port 70. In the case of FIG. 33, the exhalation port 70 is located on the patient interface 44. In this instance, the exhalation port 70 may include bias holes in the interface and/or a dedicated exhalation port 70 located on the patient interface 44. A benefit in having the exhalation port 70 in this location is that it minimizes the dead space in the circuit where CO₂ and/or other exhaled gases may accumulate and be rebreathed. In FIG. 34 the exhalation port 70 is located in the distal portion 31 of the first passageway 21 which can reduce the likelihood of the second gas 28 leaking out the exhalation port 70 without being inhaled. Although not illustrated in FIGS. 31 and 32, a non-return valve may be located in a proximal portion 30 of the second passageway 22 to inhibit the first gas 26 from passing from the first passageway 21 to the second passageway 22 during patient exhalation.

Although not illustrated in FIGS. 31 and 32, the active valve mechanism 36 may be located at the proximal portion 32 and 30 of the first and second passageways 21 and 22. In this situation a non-return valve, not illustrated, may be located at a distal portion 29 of the second passageway 22 to inhibit the second gas 28 from entering the first passageway 21. In this instance, a vent (not shown) is required in the proximal portion 30 of the second passageway 22 to discharge any residual gas in the second passageway 22 as the second gas 28 is stored therein during patient exhalation.

Another modification is the second gas inlet 35 may be located in the proximal portion 30 of the second passageway 22. In this instance, a vent (not shown) will be required to discharge any residual gas in the second passageway 22 as the second gas 28 is stored therein during patient exhalation. The vent 45 may be located at a distal portion of the second passageway 22.

In another modification, the active valve mechanism 36 and the second gas inlet 35 may be located in the proximal portion 30 of the second passageway 22. This modification avoids the need for a vent in the second passageway 22.

Although not illustrated in FIGS. 31 and 32, the circuit 20 may have a heat and moisture exchanger (HME). The HME may be in addition to, or as an alternative to a humidifier 46. The HME may be located at or near the patient interface 44, for example in passageway 23. The HME may humidify and heat the breathing gases provided to the patient interface 44. The HME may be configured to allow for bidirectional flow. In configurations with an HME, the exhalation port 70 may be located on the side of the HME opposite the patient, for example on the first passageway 21 or on the passageway 23.

FIG. 35 illustrates a breathing circuit 20 that is much the same as the breathing circuit 20 shown in FIGS. 27 and 28 in which proximal portions 32 and 30 of the first and second passageways 21 and 22 are connected to the patient interface 44. The breathing circuit 20 has an active valve mechanism 36 including first and second control valves 43 located in the distal portions 31 and 29 of the first and second passageways 21 and 22, respectively. The first and second valves 55, 56 shown in FIG. 35 may be any suitable two port valves having an inlet and an outlet. The first and second valves 55, 56 may be actively adjusted by any suitable actuator 69, such as a solenoid actuator, a pneumatic actuator, an electric motor and so forth. Each valve 55 and 56 has two ports, and in which one valve is opened while the other is closed, and vice versa, so that flow in the first and a second passageways 21 and 22 alternates depending on whether the patient in inhaling or exhaling. Specifically, during patient exhalation, the first valve 55 is opened and the second valve 56 is closed so as to supply the first gas 26 toward the patient interface 44 via the first passageway 21. In addition, it will also be appreciated that both the first and second valves 55 and 56 may be open for an overlapping period. For example, when the breathing cycle changes from patient inhalation to patient exhalation or vice versa, one of the valves 55 or 56 that is closed may move to an opened position for a period of time before the other valve 55 or 56 that is opened moves to a closed position. In addition, the second gas 28 enters the second humidification device 51 so that a volume of the second gas 28 is stored in the second humidification device 51 during patient exhalation, and depending on the volume of the second gas 28 to be stored an amount of the second gas 28 can be stored in with the second passageway 22 with a head of the second gas 28 moving along the second passageway 22 toward the patient interface 44 during patient exhalation. The second humidification device 51 may be regarded as an element of the flow assembly 24, and is, in any event an element of the breathing circuit 20. The second gas inlet 35 could be located upstream or downstream of the second humidification device 51 in the second passageway 22. To facilitate use with a sealed patient interface 44, the flow assembly includes an exhalation port 70 for venting exhaled gas from the circuit. Depending on the level of positive pressure therapy provided, the first gas 26 supplied by the flow generator 33 and the residual gas downstream of the head of the second gas 28 can be vented from circuit via the exhalation port 70. That is to say, the exhalation port 70 can also prevent overpressure of the beathing circuit 20.

During patient inhalation, the second valve 56 is opened and the first valve 55 is closed so that the second gas 28 stored in the second passageway 22 is received into the patient's airway and when the stored second gas 28 has been consumed and the patient continues to inhale, a mixture of the first and second gases 26 and 28 will be received by the patient. The second gas 28 may be supplied at a constant rate to the second passageway 22 during inhalation and exhalation.

In addition, the breathing circuit 20 comprises a humidification device 46 having first and second chambers that form part of the first and second passageways 21 and 22 respectively. The single humidification device 46 may include a dividing wall that essentially defines two isolated flow paths through each chamber so that the gases 26 and 28 in each chamber are prevented from mixing. The volume of the chamber that forms part of the second passageway 22 will contribute to the internal volume for storing the second gas 28. The flow path of the second chamber may have a volume in the range of 140 ml to 580 ml.

The humidification device 46 will also have a first chamber comprising an inlet and outlet for connection to the first passageway 21, and a second chamber comprising an inlet and outlet for connection to the second passageway 22.

Although not illustrated in FIG. 35, the circuit 20 may have a heat and moisture exchanger (HME). The HME may be in addition to, or as an alternative to a humidifier 50, 51. The HME may be located at or near the patient interface 44, for example in passageway 23. The HME may humidify and heat the breathing gases provided to the patient interface 44. The HME may be configured to allow for bidirectional flow. In configurations with an HME, the exhalation port 70 may be located on the side of the HME opposite the patient, for example on the first passageway 21 or on the passageway 23.

FIGS. 36 and 37 are simplified breathing circuits in which the at least one sensor 49 and the controller 52 have been omitted but may be included if desired, and the first and second valves 55 and 56 are both in the opened position. As explained above, during operation in most instances only one of the first or second valves 55 and 56 will be opened at any one time. However, it will also be appreciated that both the first and second valves 55 and 56 may be open for an overlapping period, for example, when the breathing cycle changes from patient inhalation to patient exhalation or vice versa. FIGS. 36 and 37 also illustrate two further locations of the exhalation port 70. In FIG. 36, the exhalation port 70 is located on the patient interface, which may for example, be bias holes in the interface and/or a dedicated exhalation port 70 located on the patient interface 44. A benefit in having the exhalation port 70 in this location is that it minimizes the dead space in the circuit where CO₂ and/or other exhaled gases may accumulate and be rebreathed. The exhalation port 70 in FIG. 37 is located in the distal portion 31 of the first passageway 21 which can reduce the likelihood of the second gas 28 leaking out the exhalation port 70 without being inhaled.

Although not shown in FIG. 35, the flow assembly 24 may include a non-return valve in the proximal portion 30 of the second passageway 22 to inhibit the first gas 26 from entering the second passageway 22.

In another configuration, the active valve mechanism 36 of the breathing circuit 20 can be located in proximal portions 32 and 30 of the first and second passageways 21 and 22.

In another configuration, the second gas inlet 35 can be located in the proximal portion 30 of the second passageway 22. This configuration is possible with the active valve mechanism 36 located either in the proximal portion 32 and 30 or the distal portion 31 and 29 of the passageways 21 and 22. In any event, when the second gas inlet 35 is located in the proximal portion 30 of the second passageway 22, a venting valve (not illustrated) may be required in the distal portion 29 of the second passageway 22 to allow residual gases to be vented during patient exhalation and, in turn, allow a volume of the second gas 28 to be stored in the second passageway 22.

FIG. 38 illustrates a breathing circuit 20 that is similar to the circuit 20 shown in FIGS. 27, 28, 31 and 32 and may include the features described in the relation to these Figures. In the case of FIG. 38, the first and second passageways 21 and 22 of the breathing circuit 20 comprises distal portions 31 and 29 only. An active valve mechanism 36 is provided in the distal portions 31 and 29 of the passageways 21 and 22, and the first gas source 25 supplies the first gas 26 to the passageways 21 and 22. A second gas inlet 35 is connected to a second chamber of a dual chamber humidification device 46. These features and others of the breathing circuit 20 shown in FIG. 38 can be equated to the features shown in FIGS. 27, 28, 31 and 32.

As can be seen, the first and second passageways 21, 22 merge together to provide a third passageway 23 having a proximal portion that connects to a patient interface 44.

If the active valve mechanism 36 is operated to open the second valve 56 at the start of patient inhalation, residual gases in the third passageway 23 may delay the onset of the stored second gas 28 reaching the patient. The delay may be proportional to the volume of the third passageway 23.

As the internal volume of the third passageway 23 may be a noticeable portion of the tidal volume of the patient, it may be desirable to control the second valve 56 to compensate for this delay. That is, if the respiratory rate is known, the beginning of inhalation can be predicted, and the active valve mechanism 36 can be operated to start flow of the first gas 26 to the second passageway 22 for a period prior to patient inhalation. The second gas 28 can then reach the patient as inspiration begins. To help minimise the delay, the breathing circuit 20 includes at least one sensor 49 that detects the breathing cycle of the patient 74, namely when the patient is inhaling and exhaling.

The at least one sensor 49 may be located anywhere within the breathing circuit 20 or on the patient 74, as represented by the dashed line leading to the sensor 49 in FIG. 38. For example, the at least sensor 49 may be located on either, the patient interface 44, the first passageway 21, the second passageway 22, on the flow generator 33, or when multiple sensors 49 are provided, the sensors 49 may be provided on any combination of these. In addition to gas flow rate, the sensor 49 may measure temperature, gas pressure or gas composition to detect the breathing cycle of the patient. The controller 52 may receive an output signal of the sensor(s) 49, and the controller 52 calculates, or has a process that calculates the period of inhalation and/or exhalation, and in turn, produces a control output signal that is used to operate the active valve mechanism 36. Alternatively, the sensor(s) 49 may determine whether the patient is inhaling or exhaling and the controller 52 produces a control output signal that operates the active valve mechanism 36 to supply the first gas 26 or the second gas 28 depending on the output signal of the sensor(s) 49. In any event, the controller 52 may calculate the period of inhalation, and generate a control output signal to operate the active valve mechanism 36 that minimizes the delay between the active valve mechanism 36 supplying the first gas 26 to the second passageway 22 and the patient receiving the breathing gas from the second passageway 22. In other words, the internal volume of the third passageway 23 and the humidification device 46, if present, can delay the onset of the second gas 28 being supplied to the patient, and the controller can reduce the delay by generating an output signal to operate the active valve mechanism 36 to pre-empt patient breathing.

To facilitate use with a sealed patient interface 44, the flow assembly includes an exhalation port 70 for venting exhaled gas from the circuit 20. Depending on the level of positive pressure therapy provided, the first gas 26 supplied by the flow generator 33 and the residual gas downstream of the head of the second gas 28 can be vented from circuit via the exhalation port 70. That is to say, the exhalation port 70 can also prevent overpressure of the beathing circuit 20.

Although not illustrated in FIG. 38, the circuit 20 may have a heat and moisture exchanger (HME). The HME may be in addition to, or as an alternative to a humidifier 50, 51. The HME may be located at or near the patient interface 44, for example in passageway 23. The HME may humidify and heat the breathing gases provided to the patient interface 44. The HME may be configured to allow for bidirectional flow. In configurations with an HME, the exhalation port 70 may be located on the side of the HME opposite the patient, for example on the first passageway 21 or on the passageway 23.

FIGS. 39 and 40 are simplified breathing circuits in which the at least one sensor 49 and the controller 52 have been omitted for simplicity, but may be included if desired, and the first and second valves 55 and 56 are both in the opened position. As explained above, during operation only one of the first or second valves 55 and 56 will be opened at any one time. FIGS. 39 and 40 also illustrate two further locations of the exhalation port 70. In FIG. 39, the exhalation port 70 is located on the patient interface, which may for example, be bias holes in the interface and/or a dedicated exhalation port 70. A benefit in having the exhalation port in this location is that it minmises the dead space in the circuit where CO₂ and/or other exhaled gases may accumulate and be rebreathed. The exhalation port 70 in FIG. 40 is located in the distal portion 31 of the first passageway 21 which can reduce the likelihood of the second gas 28 leaking out the exhalation port 70 without being inhaled.

FIG. 41 illustrates a breathing circuit 20 that is similar to the circuit 20 shown in FIGS. 27, 28 and 35 in which distal portions 31, 29 includes first and second passageways 21 and 22 that are connected to a first gas source 25 such as air flow generator 33 as illustrated, and an active valve mechanism 36 including two separate control valves 55 and 56 is provided in the distal portions 31 and 29 of the passageways 21 and 22. These features and others of the circuit 20 shown in FIG. 41 can be equated to the features shown in FIGS. 27, 28 and 35.

As can be seen, the first and second passageways 21 and 22 merge together to provide a third passageway 23 having a proximal portion that connects to a patient interface 44. The flow assembly includes a humidification device 46 located on the third passageway 23 such that all of the breathing gas supplied to the patient interface 44 is substantially humidified uniformly.

The flow assembly 24 also includes a reservoir 47 located in the second passageway 22 to provide the second passageway 22 with the required internal volume. Although not illustrated in the figures, the internal volume of the reservoir 47 may be adjustable, which in turn adjusts the volume of the second gas 28 stored in the second passageway 22 during patient exhalation. The internal volume may be selected based on the tidal volume of the patient. Adjustments made to the volume of the reservoir 47 may also need to be taken into account by adjusting the flow rate of the second gas 28 supplied to the reservoir 47. In addition, the second gas inlet 35 may be located upstream of the reservoir 47. In any event, as the second gas 28 enters the second passageway 22, a head of the second gas 28 moves toward the patient interface 44 and residual gases downstream of the head can be discharged from the circuit 20 by the patient interface 44.

Although FIG. 41 illustrates the first and second valves 55 and 56 in the opened position, only one of the first and second valves 55 and 56 would be opened at any time during use of the breathing circuit 10. Specifically, during patient exhalation, the first valve 55 is opened and the second valve 56 is closed so as to supply the first gas 26 to the patient interface 44 via the first passageway 21. During patient inhalation, the second valve 56 is opened and the first valve 55 is closed so that the second gas 28 stored in the second passageway 22 is received into the patient's lungs and when the stored second gas 28 has been consumed and the patient continues to inhale, a mixture of the first and second gases 26 and 28 will be received by the patient. The second gas 28 may be supplied at a constant rate to the second passageway 22 during inhalation and exhalation. It is also possible that the second gas 28 could be supplied at a variable rate to the second passageway 22. If the active valve mechanism 36 is operated to open the second valve 56 at the start of patient inhalation, residual gases in the third passageway 23 may delay the onset of the stored second gas 28 reaching the patient. The delay between the first gas 26 being supplied to the second passageway 22 by the second valve 56 opening and the stored second gas 28 reaching the patient is proportional to the volume of the third passageway 23. In the case of FIG. 41, the third passageway 23 will have a length of tubing that minimises weight or pull on the patient interface. As the internal volume of the third passageway 23 may be significant to a particular patient's tidal volume, it may be desirable to control the second valve 56 to compensate for this delay. To help take this into account, the breathing circuit 20 includes at least one sensor 49 that detects the breathing cycle of the patient, namely when the patient is inhaling and exhaling.

The at least one sensor 49 may be located anywhere within the breathing circuit 20 or on the patient 74, as represented by the dashed line leading to the sensor 49 in FIG. 41. For example, the at least sensor 49 may be located on either, the patient interface 44, the first passageway 21, the second passageway 22, on the flow generator 33, or when multiple sensors 49 are provided, the sensors may be provided on any combination of these. In addition to gas flow rate, the sensor 49 may measure temperature, gas pressure or gas composition to detect the breathing cycle of the patient. The controller 52 may receive an output signal of the sensor(s) 49, and the controller 52 calculates, or has a process that calculates the period of inhalation and/or exhalation, and in turn, produces a control output signal that is used to operate the active valve mechanism 36. Alternatively, the sensor(s) 49 may determine whether the patient 74 is inhaling or exhaling and the controller 52 produces a control output signal that operates the active valve mechanism 36 to supply the first gas 26 or the second gas 28 depending on the output signal of the sensor(s) 49. In any event, the controller 52 may calculate the period of inhalation, and generate a control output signal to operate the active valve mechanism 36 that minimizes the delay between the active valve mechanism 36 supplying the first gas 26 to the second passageway 22 and the patient receiving the breathing gas from the second passageway 22. In other words, the internal volume of the third passageway 23 and the reservoir 47, can delay the onset of the second gas 28 being supplied to the patient, and the controller 52 can reduce the delay by generating an output signal to operate the active valve mechanism 36 to pre-empt patient breathing.

To facilitate use of the breathing circuit 20 shown in FIG. 41 with a sealed patient interface 44, the flow assembly also includes an exhalation port 70 for venting exhaled gas. The exhalation port can also be used for venting residual breathing gas from the third passageway 23 when the second valve 56 is opened, for instance in anticipation of the patient inhaling. Similarly, the exhalation port 70 can also be used for venting the first gas 26 from the first passageway 21 when the first valve is opened during patient exhalation.

Although not illustrated in FIG. 41, the circuit 20 may have a heat and moisture exchanger (HME). The HME may be in addition to, or as an alternative to a humidifier 46. The HME may be located at or near the patient interface 44, for example in passageway 23. The HME may humidify and heat the breathing gases provided to the patient interface 44. The HME may be configured to allow for bidirectional flow. In configurations with an HME, the exhalation port 70 may be located on the side of the HME opposite the patient, for example on the first passageway 21 or on the passageway 23.

FIGS. 42 and 43 are simplified breathing circuits 20 in which the at least one sensor 49 and the controller 52 have been omitted for simplicity of the Figures, and the first and second valves 55 and 56 are both in the opened position. As explained above, during operation only one of the first or second valves 55 and 56 will be opened at any one time. FIGS. 42 and 43 also illustrate two further locations of the exhalation port 70. In FIG. 42, the exhalation port 70 is located on the third passageway 23. A benefit in having the exhalation port 70 in this location is that it reduces the dead space in the circuit where CO₂ and/or other exhaled gases may accumulate and be rebreathed. The exhalation port 70 in FIG. 43 is located in the distal portion 31 of the first passageway 21 which can reduce the likelihood of the second gas 28 leaking out the exhalation port without being inhaled.

One of the characteristics of the circuits 20 shown FIGS. 15 to 43 is that during exhalation, any leak from the circuit 20, such as unintentional mask leak, or intentional leak through the exhalation port 70 or bias hole, is unlikely to comprise high concentrations of the second gas 28, or at least concentrations of the second gas 28 that have not already been inhaled. Rather, gas vented from the exhalation port 70 will include the exhaled gas and first gas 26 conveyed through the first passageway 21. In addition, the exhalation port 70 may include some residual gas that has not been breathed, such as residual gas from the dead space of the circuit 20. This improves the efficiency of use the second gas 28.

Although not shown in any Figures, one or more filters may also be located in the first and second passageways 21 and 22 between the first and second valves 55 and 56 and the patient interface 44.

Although not shown in the Figures, the first and second passageways 21, 22 may include tubing of any suitable structure. For example, the first and second passageways 21, 22 may be separate tubes. The tubes may be unconnected along their length, or they may be connected side-by-side using connector clips, a permanent adhesive, or be integrally formed. An integrally formed structure may be extruded.

The first and second passageways 21, 22 may be provided at least in part by a multi-lumen tube, in which separate lumens provide the first and the second passageways 21, 22. For example, the structure of a multi-lumen tube may have side-by-side passageways, in which a partition along the tube defines in part the first and second passageways 21, 22 along the tube. The multi-lumen tube having side-by-side passageways may be extruded. In another example, the structure of the multi-lumen tube may be a coaxial structure, in which one passageway is arranged centrally, and the other passageway is arranged about the periphery of the central passageway.

FIGS. 7, 8, 11, 12, 13, 27 to 30, 35 to 43 all illustrate the active valve mechanism 36 adjacent to the flow generator 33. Although not specifically shown in these figures, the active valve mechanism 36 may also be incorporated with the respective flow generator 33, such as being integrated within a housing of the flow generator 33.

In yet another example, the first and second passageways 21, 22 may be arranged as a single conduit formed from a spirally wound hollow body. The conduit may comprise a first elongate member having a hollow body spirally wound to form at least in part an elongate tube having a hollow wall surrounding the conduit lumen. The conduit may also include a second elongate member spirally wound and joined between adjacent turns of the first elongate member. The spirally wound hollow body may provide either one of the first and the second passageways, and the conduit lumen formed by the spirally wound hollow body provides the other tube.

The spirally wound hollow body may provide a smaller internal volume than the conduit lumen. In some situations, it is desirable that the flow rate provided during exhalation be smaller than the flow rate provided during inhalation. In this example, the spirally wound hollow body may provide the first passageway 21, and the conduit lumen may provide the second passageway 22. In another example, the spirally wound hollow body may provide the second passageway 22, and the conduit lumen may provide the first passageway 21.

Alternatively, the conduit lumen may be the second passageway 11 and the second gas inlet 35 may be provided into the spirally wound hollow body. The second gas 28 may enter a distal portion of the spirally wound hollow body, flow along the spirally wound hollow body towards the patient, and then flow into a proximal portion of the conduit lumen. This effectively allows a proximal second gas inlet 35 without needing an additional conduit near the patient interface.

Examples of conduits comprising spiral wound hollow bodies are disclosed in International patent publication WO2012/164407 (PCT/IB2012/001786) entitled MEDICAL TUBES AND METHODS OF MANUFACTURE filed 30 May 2012, the full contents of which are hereby incorporated into this specification.

As mentioned above, the flow generator 33 may be a blower. In another example, not illustrated the flow generator 33 may be, or include, a flow regulator for controlling the flow rate and or pressure of the first gas conveyed along the first and second passageways 21 and 22. The flow regulator may be the flow generator 43 and a flow valve, or an actively controlled valve.

Although not illustrated in FIG. 1 to 14, the circuit 20 may have a heat and moisture exchanger (HME). The HME may be in addition to, or as an alternative to a humidifier. The HME may be located at or near the patient interface 44, for example in passageway 23. The HME may humidify and heat the breathing gases provided to the patient interface 44. The HME may be configured to allow for bidirectional flow. In configurations with an HME, the exhalation port 70 may be located on the side of the HME opposite the patient, for example on the first passageway 21 or on the passageway 23.

FIG. 44 is a block diagram summarising the elements of the breathing circuits 20 shown in FIGS. 1 to 43. The breathing circuits 20 include first and second passageways 21, 22 for supply breathing gas to the patient interface 44 and in turn to the patient, and elements that have been collectively referred to as a flow assembly 24, identified by the inner dashed box that regulates the flow of first and second gas 26, 28 as the breathing gas to the passageways 21, 22. The arrows and lines between the elements represent relationships and associations between the elements, which could represent, but not necessarily, the flow of breathing gas between the elements or transmission of the output signals between the elements. The flow assembly 24 includes a first gas source 25 and a second gas source 27, an active valve assembly 36 that regulates flow from the first gas source 25 to the first passageway 21 and to the second passageway 22. The flow assembly 36 may also include at least one sensor 49 for detecting the breathing cycle of a patient. The sensor(s) 49 may detect a parameter of a gas flow such as flow rate, pressure, temperature and gas composition at any point in the breathing circuit 20, including the patient interface 44, first and second passageways 21 and 22, or at the first gas source 25. The sensor(s) 49 may also be external sensor(s) fitted directly onto the abdomen, neck, chest or other parts of a patient to detect patient inhalation and exhalation. Alternatively, the sensor(s) 49 may be non-contact sensors. An output signal from the sensor 49 may be received directly by the active valve mechanism 36 which in turn regulates the flow from the first gas source 25 to the first and second passageways 21 and 22, or via a controller. In any event, the first gas source 25 supplies flow to the first passageway 21 by the active valve mechanism 36 during patient exhalation, and the first gas source 25 supplies flow to the second passageway 22 by the active valve mechanism 36 during patient inhalation. The second gas source 27 supplies flow to the second passageway 22 during patient inhalation and exhalation and flow from the second gas source 27 to the second passageway 22 may be controlled at a constant rate or varied depending on the requirements. During patient inhalation, any residual breathing gas not inhaled will be vented from the second passageway 22 by the active valve mechanism 36 closing the second passageway to the patient interface 44, and the second gas 28 entering the second passageway 22 displacing the residual gas through a vent 45.

A controller 52 may receive an output signal of the sensor(s) 49, and the controller 52 calculates, or has a process that calculates the period of inhalation and/or exhalation, and in turn, produces a control output signal that is used to operate the active valve mechanism 36. Alternatively, the sensor(s) 49 may determine whether the patient is inhaling or exhaling and the controller 52 provides a control output signal that can be used to operate the active valve mechanism 36 to supply the first gas 26 or the second gas 28 depending on the output signal of the sensor(s) 49.

In addition, the controller 52 may calculate the period of inhalation, and generate a control output signal to operate the active valve mechanism 36 that minimizes the delay between the active valve mechanism 36 supplying the first gas 26 to the second passageway 22 and the patient receiving the breathing gas from the second passageway 22. This can be advantageous when the breathing circuit 23 has the third passageway 23 and optionally a humidification device 46 in the third passageway 23. In other words, the internal volume of the third passageway 23 and the humidification device 46, if present, can delay the onset of the second gas 28 being supplied to the patient, and the controller can reduce the delay by generating an output signal to operate the active valve mechanism 36 to pre-empt patient breathing.

Other elements, such as humidification devices 46, a reservoir 47 in the second passageway 22, non-return valves and so forth described above may be included in the breathing circuit 20, but have not been illustrated in FIG. 10 to maintain clarity.

The breathing circuits 20 include a number of features in common described above, plus a number of further in common features that are now described. For instance, the patient interface 44 may be an unsealed patient interface 44 or a sealed patient interface 44. Examples of unsealed interfaces 44 include: nasal cannula, a tracheostomy interface/tube that are inserted into the neck of a patient, an oral mask that allows venting through the nasal passage, a sealed nasal mask that allows venting through the mouth, an unsealed face mask, or a face mask that has an exhaust port. Examples of sealed interfaces 44 include: a full-face mask (also known as an oro-nasal mask), a sealed nasal cannula, a sealed oral mask, a sealed nasal mask, a nasal pillows interface, or a tracheostomy member. When a sealed breathing circuit 20 is used, the breathing circuit may also include the exhalation port 70 as shown in FIGS. 15 to 43.

The first gas 26 may be any suitable gas such as pressurized air, or pressurized air enriched with oxygen. The flow generator 33 may be any blower, fan and so forth, and may have a filter for filtering the first gas 26. Accordingly, the first gas 26 may be ambient air that has been filtered by the flow generator 33. However, filtering the first gas 26 is optional. Therefore, all of the embodiments disclosed here may utilize a first gas 26 that is unfiltered. It follows that the embodiments may involve the first gas 26 being unfiltered ambient air. The flow generator 33 may be a flow regulator for controlling the flow of the first gas 26. For instance the flow regulator may be the flow generator 33 and a valve, such as a control volve or an actively controlled valved for controlling the flow of the first gas.

The second gas 28 may be any pressurized gas including one or any combination of: oxygen gas, heliox, or an anaesthetic gas. The anaesthetic gas could be nitrous oxide or a 50:50 mixture of nitrous oxide and oxygen gas. For example, pressurized oxygen gas may be supplied from a liquified oxygen source, a bottled oxygen source or from an oxygen concentrator source.

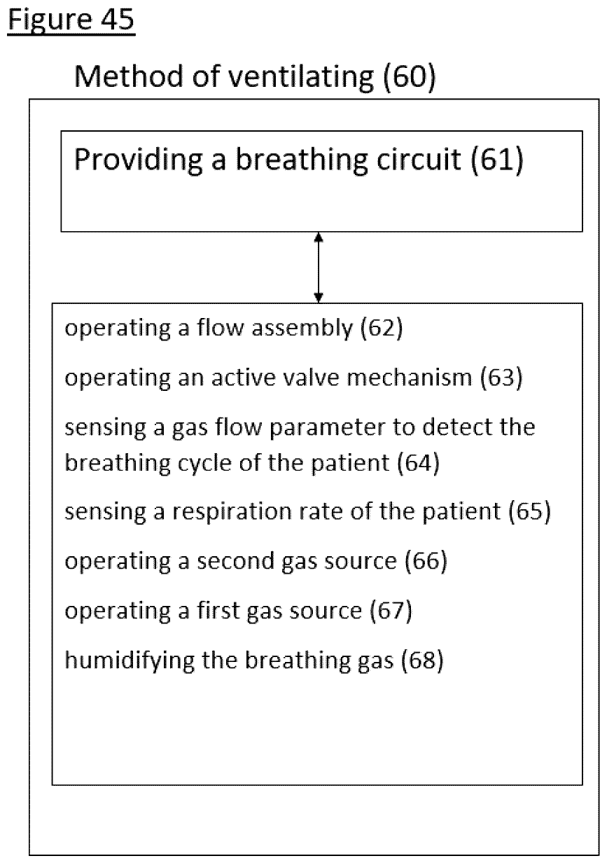

FIG. 45 is a block diagram of method 60 for providing respiratory support to a patient a patient. The method 60 may be carried out using the breathing circuit 20 shown and described herein. The method may include supplying a first gas to a first passageway connected to a patient interface during patient exhalation, and supplying a second gas to a second passageway connected to the patient interface. The step of supplying the first gas to the second passageway displaces at least some of the second gas in the second passageway to the patient interface during patient inhalation. The method 60 may also providing a breathing circuit 20 having first and second passageways 21 and 22 that convey a breathing gas to a patient interface 44, and a flow assembly 24 that is connectable to a first passageway 21 and is connectable to a second passageway 22. The breathing circuit 20 may include any one of the other elements of the breathing circuits 20 described herein including a humidifier, non-return valves, sensor(s), a controller, flow generators and so forth. The method 60 will also include operating the flow assembly 62 so that first passageway 21 supplies the first gas 26 to the patient interface 44 during patient exhalation, and the second passageway 22 supplies the first gas 26 and the second gas 28 to the patient interface 44 during patient inhalation.

The method may include operating an active valve mechanism 36 to allow or inhibit the flow of a first gas 26 to the first and second passageways 21, 22. Typically, this will involve alternating flow of the first gas 26 between the first passageway 21 and the second passageway 22 during patient exhalation and inhalation. However, it will be appreciated that the first passageway 21 may have a reduced flow of the first gas 26, instead of no flow, during patient exhalation compared to flow of the first gas during patient inhalation. Similarly, the second passageway 22 may have a reduced flow of the first gas 26, instead of no flow of the first gas 26, during patient exhalation compared to the flow of the first gas during patient inhalation.

The method may include sensing a gas flow parameter 64, such as flow rate and/or pressure, in the breathing circuit 20 to detect the breathing cycle of the patient. The active valve mechanism 26 can then be operated to supply flow of the first gas 26 to the first passageway 21 during patient exhalation and to supply flow of the first gas 26 to the second passageway 22 during patient inhalation based on an output of the sensor 49.

The method may include sensing a respiration rate 65 of the patient, for example by using a sensor fitted to a patient's abdomen. An output of the sensor 49 can then be used to operate the active valve mechanism 36 to supply flow of the first gas 26 to the first passageway 21 during patient exhalation, and to supply flow of the first gas 26 to the second passageway 22 during patient inhalation.

The active valve mechanism 36 may be operated so that the first gas 26 is supplied to the first passageway 21 at the start of the exhalation and to supply the first gas 26 to the second passageway 22 at the start of inhalation, respectively. However, if the breathing circuit 20 has a delay in supplying the breathing gas after the first gas 26 has been supplied to the second passageway 22, for example due to internal volume of one or more of the components of the flow assembly 62 and the passageways 21 and 22, and in particular the third passageway 23, operating the flow assembly 24 may include controlling the active valve mechanism 36 to minimise any delay in supplying the second gas 28 to the patient interface 44. Specifically, at the start of inhalation by operating the active valve mechanism 36 to supply the first gas 36 to the second passageway 22 before inhalation by a pre-emptive period so the breathing gas is supplied by the second passageway 22 at the start of patient inhalation. This may for example, be relevant to breathing circuits such as those shown in FIGS. 7 and 8 having a third passageway 23.

The step of operating the flow assembly 62 may include operating a second gas source 66 by controlling the flow rate of the second gas 28 into the second passageway 22. Similarly, the step of operating the flow assembly 62 may include operating a first gas source 67, including a flow generator 33, to supply the first gas 26 to the first and the second passageways 21 and 22.

The step of operating the flow assembly 62 may include operating a single flow generator 33 to supply the first gas 26.

The step of operating the flow assembly 62 may include operating first and second flow generators, in which the first flow generator supplies a stream of the first gas to the first passageway and the second flow generator supplies the first gas to the second passageway 22.

The method described herein may include any one or a combination of the other elements described herein. For example the method may include: humidifying the breathing gas using a humidification device, controlling the flow rate of the breathing gas including controlling the flow rate of the respective first and second gases, providing a non-return valve in the first and/or second passageways, and so forth.

Conditional language used herein, such as, among others, “can,” “might,” “may,” “for example,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or methods illustrated can be made without departing from the spirit of the disclosure. As will be recognized, certain embodiments of the inventions described herein can be embodied within a form that does not provide all of the features and benefits set forth herein, as some features can be used or practiced separately from others.

### Statements

1. A breathing circuit for providing respiratory support to patient, the breathing circuit comprising:
   first and second passageways that can convey a breathing gas to a patient interface, wherein the first passageway is connectable to a first gas source to supply a first gas, and the second passageway is connectable to the first gas source and to a second gas source, the second gas source to supply the second gas, and
   wherein the first passageway can convey the first gas during patient exhalation and the second passageway supply the first gas and the second gas during patient inhalation.
2. The breathing circuit according to statement 1, wherein the first and second passageways can convey the breathing gas to the patient interface at all times, and can convey the second gas to the patient interface independently of the first gas being conveyed to the patient interface by the first passageway.
3. The breathing circuit according to statement 1 or 2, wherein the breathing circuit comprises the first gas source that supplies the first gas.
4. The breathing circuit according to any one of the preceding statements, wherein the first gas source comprises a flow generator that generates a flow of the first gas.
5. The breathing circuit according to any one of the preceding statements, wherein the breathing circuit comprises the second gas source that supplies the second gas, the second gas source being a pressurized gas source.
6. The breathing circuit according to statement 5, wherein the pressurized gas includes one or any combination of: oxygen gas, heliox, or an anaesthetic gas.
7. The breathing circuit according to statement 5 or 6, wherein the second gas source supplies the second gas to the second passageway during patient inhalation.
8. The breathing circuit according to any one of statements 5 to 7, wherein the second gas source supplies the second gas to the second passageway during patient exhalation.
9. The breathing circuit according to any one of statements 5 to 8, wherein the second gas source the second gas to the second passageway during patient inhalation and during patient exhalation.
10. The breathing circuit according to any one of the preceding statements, wherein the breathing circuit is operable to adjust the flow of the first gas in the first and the second passageways based on the breathing cycle of the patient, that is, based on patient inhalation and patient exhalation.
11. The breathing circuit according to any one of the preceding statements, wherein the breathing circuit is operable to alternate flow of the first gas between the first passageway and the second passageway.
12. The breathing circuit according to any one of the preceding statements when appended to statement 4, wherein the breathing circuit is operable to adjust a parameter of the first gas in the first passageway, including the flow generator being operable to adjust the flow rate of the first gas supplied to either one or both of the first passageway and the second passageway to provide high flow therapy.
13. The breathing circuit according to any one of the preceding statements when appended to statement 4, wherein the breathing circuit is operable to adjust a parameter of the first gas in the first passageway, including the flow generator being operable to adjust the pressure of the first gas supplied to either one of, or both of, the first passageway and the second passageway to provide CPAP therapy or bi-level pressure therapy.
14. The breathing circuit according to any one of the preceding statements when appended to statement 4, wherein the flow generator is connectable to the first and second passageways to divide flow from the flow generator to the first and the second passageways.
15. The breathing circuit according to any one of the preceding statements when appended to statement 4, wherein the breathing circuit is operable to alternate flow of the first gas from the flow generator between the first passageway and the second passageway.
16. The breathing circuit according to statement 14 or 15, wherein the alternate flow of the first gas comprises there being:
   iii) substantially no, or little flow, of the first gas in the first passageway during inhalation, and there being flow of the first gas in the second passageway during inhalation, and
   iv) substantially no or little flow of the first gas in the second passageway during exhalation, and there being flow of the first gas in the first passageway during exhalation.
17. The breathing circuit according to any one of the preceding statements, wherein the breathing circuit comprises one or more valves to adjust the flow of the first gas in the first passageway and the second passageway.
18. The breathing circuit according to statement 17, wherein the one or more valves alternate the flow of the first gas between the first passageway and the second passageway.
19. The breathing circuit according to statement 17 or 18, wherein the one or more valves comprises two valves in which one valve is located in the first passageway, and a second is located on the second passageway.
20. The breathing circuit according to any one of statements 17 to 19, wherein the one or more valves comprises a three-way valve having one inlet connectable to the first gas source and two outlets, in which one of the outlets is connectable to the first passageway and the second outlet is connectable to the second passageway.
21. The breathing circuit accord to any one of statement 17 to 19, wherein the one or more valves includes an active valve mechanism.
22. The breathing circuit according to any one of the preceding statements, wherein the breathing circuit comprises an active valve mechanism to adjust the flow of the first gas in the first passageway and the second passageway.
23. The breathing circuit according to statement 21 or 22, wherein the active valve mechanism is operable to alternate flow of the first gas between the first passageway and the second passageway so that the first gas can be delivered to the patient interface via the first or the second passageways at any one time.
24. The breathing circuit according to any one of statements 21 to 23, wherein the active valve mechanism comprises an actively-controlled valve that is operated by an actuator that receives an output signal from a sensor that sensors the breathing cycle of the patient.
25. The breathing circuit according to any one of statements 21 to 24, wherein the active valve mechanism is located in distal portions of the first and second passageways.
26. The breathing circuit according to any one of statements 21 to 24, wherein the active valve mechanism is located in proximal portions of the first and second passageways.
27. The breathing circuit according to any one of statements 21 to 26, wherein the active valve mechanism comprises an actively-controlled valve comprising a three-way valve to alternate flow of the first gas between the first passageway and the second passageway.
28. The breathing circuit according to statement 27, wherein the three-way valve is connected to proximal portions of the first and second passageways and has a first port that is connectable to the first passageway, a second port that is connectable to the second passageway, and a third port that is connectable to the patient interface.
29. The breathing circuit according to statement 27, wherein the three-way valve is connected to a distal portions of the first and second passageways and has a first port that is connectable to the first passageway, a second port that is connectable to the second passageway, and third port that is connectable to a first gas source.
30. The breathing circuit according to any one of statements 21 to 27, wherein the active valve mechanism comprises first and second control valves, in which the first control valve is located in the first passageway and the second control valve is located in the second passageway to control flow of the first gas to the passageways.
31. The breathing circuit according to statement 30, wherein the first and second control valves are opened and closed alternately, that is, the first control valve opened and the second control valve closed during patient exhalation, and the first control valve closed and the second control valve opened during patient inhalation.
32. The breathing circuit according to any one of the preceding statements, wherein the breathing circuit includes a third passageway interconnecting the patient interface and a joiner that merges the first and the second passageways.
33. The breathing circuit according to any one of preceding statements when appending to statement 4, wherein the flow generator is a single blower.
34. The breathing circuit according to any one of preceding statements when appending to statement 4, wherein the flow generator has a single flow output that is split between the first and the second passageways.
35. The breathing circuit according to any one of preceding statements when appending to statement 4, wherein the flow generator has an output that is split between the first passageway and the second passageways.
36. The breathing circuit according to any one of preceding statements when appending to statement 4, wherein the flow generator has two flow outputs, and each flow output can be delivered to either one of the respective first and second passageways.
37. The breathing circuit according to statement 36, wherein each flow output is driven by a separate blower.
38. The breathing circuit according to any one of preceding statements when appending to statement 4, wherein the flow generator comprises a first flow generator that conveys the first gas along the first passageway, and a second flow generator that conveys the first gas along the second passageway.
39. The breathing circuit according to any one of the preceding statements, wherein the breathing circuit further comprises a sensor for detecting the breathing cycle of the patient, that is, when patient inhalation and patient exhalation is occurring, and the sensor has an output signal that is used to operate the active valve mechanism.
40. The breathing circuit according to statement 39, wherein the sensor has an output signal that is used to operate the flow generator.
41. The breathing circuit according to statement 40, wherein the output signal can be used to adjust the flow rate of the first gas supplied by the flow generator for high flow therapy.
42. The breathing circuit according to statement 40, wherein the output signal can be used to adjust the pressure of the first gas supplied by the flow generator for CPAP or bi-level pressure therapy.
43. The breathing circuit according to statement 40, wherein the output signal can be used to adjust the pressure of the first gas supplied by the flow generator between a first pressure being an IPAP (inspiratory positive airway pressure) and a second pressure being an EPAP (expiratory positive airway pressure).
44. The breathing circuit according to any one of statements 39 to 43, wherein the sensor comprises at least one gas sensor that can detect at least one of: i) a gas property of the breathing gas supplied to the patient interface, ii) a gas property of exhaled gases, and/or iii) a gas property of gases being vented from the breathing circuit.
45. The breathing circuit according to any one of statements 39 to 44, wherein the sensor comprises comprises multiple sensors located at a selection of the following locations: i) the patient interface; ii) an outlet of the flow generator, iii) in the first passageway, and iv) in the second passageway.
46. The breathing circuit according to statement 45, wherein the gas property comprises at least one of: gas flow rate, gas pressure, gas temperature, gas humidity or gas concentration, such as oxygen or carbon dioxide concentration.
47. The breathing circuit according to any one of statements 39 to 46, wherein the sensor includes a pressure sensor located at or near an outlet of the flow generator, or located on the patient interface.
48. The breathing circuit according to any one of statements 39 to 47, wherein the sensor includes a pressure sensor at the second gas inlet, or at a vent in the first passageway, or at an exhalation portion.
49. The breathing circuit according to any one statements 39 to 48, wherein the sensor includes a flow sensor located at one or more of the following: i) at or near an outlet of the flow generator, ii) at or near the patient interface; iii) at the second gas inlet.
50. The breathing circuit according to any one of statements 39 to 49, wherein the sensor includes a flow sensor located in one or more the following: i) in the first passageway, ii) in the second passageway, or iii) an exhalation vent.
51. The breathing circuit according to any one of statements 33 to 38 when appended to any one of statements 21 to 31, wherein an output signal of the sensors is used to determine if the patient is inhaling or exhaling and, in turn, control the active valve mechanism so that the first gas can be delivered to the first passageway during exhalation and the first gas delivered to the second passageway during inhalation at a desired flow rate or a desired pressure.
52. The breathing circuit according to any one of statements 39 to 51, wherein the sensor comprises an external sensor that detects a respiratory parameter of the patient.
53. The breathing circuit according to any one of statements 39 to 52, wherein the breathing circuit further comprises a controller that receives the output signal of the sensor(s), and the controller has a processor that calculates the period of inhalation and/or exhalation, and in turn, produce a control output that is used to operate the active valve mechanism.
54. The breathing circuit according to statement 53, wherein the controller calculates the period of inhalation, and generates an output signal that used to operate the active valve mechanism that minimizes the delay between the active valve mechanism delivering the first gas to the second passageway and the patient receiving the breathing gas from the second passageway at the start of patient inhalation.
55. The breathing circuit according to any one of statements 53 or 54 when appended to any one of statements 4, or 33 to 38, wherein the flow generator is adjusted to control the flow rate of the first gas supplied to the first and second passageways based on the output signal of the controller for high flow therapy.
56. The breathing circuit according to any one of statements 53 or 54 when appended to any one of statements 4, or 33 to 38, wherein the flow generator is adjusted to control the pressure of the first gas supplied to the first and second passageways based on the output signal of the controller for CPAP or bi-level therapy.
57. The breathing circuit according to any one of the preceding statements, wherein the breathing circuit comprises the patient interface which is an unsealed patient interface for high flow therapy.
58. The breathing circuit according to any one of statements 1 to 56, wherein the breathing circuit comprises the patient interface which is a sealed patient interface for CPAP therapy or bi-level pressure therapy.
59. The breathing circuit according to any one statements 1 to 57, wherein the breathing circuit comprises an vent on the second passageway for venting part or all of a residual breathing gas from the second passageway during patient exhalation.
60. The breathing circuit according to statement 59, wherein the vent is located downstream of the where the second gas enters the second passageway, so that the second gas entering the second passageway during patient exhalation displaces the residual gas though the vent, in which the breathing gas displaced through the vent comprises any of the first and/or the second gas in the second passageway not inhaled.
61. The breathing circuit according to statement 59 or 60, wherein the vent comprises any one or a combination of the control valve, PEEP (positive end-expiratory pressure) valve, an aperture of fixed size, or a controlled outlet that maintains a substantially constant venting flow across a range of pressures.
62. The breathing circuit according to statement 58, wherein the breathing circuit comprises an exhalation port for venting exhaled gas from the breathing circuit.
63. The breathing circuit according to statement 62, wherein the breathing circuit comprises an exhalation port for venting first gas from the first passageway to prevent over-pressurising the patient interface.
64. The breathing circuit according to statement 63, wherein the exhalation port is located on the patient interface.
65. The breathing circuit according to statement 63, wherein the exhalation port is located on a proximal portion of the first passageway, which reduces the likelihood of the second gas being vented through the exhalation port without being inhaled by the patient.
66. The breathing circuit according to statement 63, wherein the exhalation port is located on a distal portion of the first passageway, which reduces the likelihood of the second gas inadvertently being leaked from the circuit.
67. The breathing circuit according to any one of statements 63 to 66, wherein the second passageway includes a non-return valve to inhibit any residual amount of the breathing gas not inhaled with each breath from flowing upstream, that is, in a direction opposite to the direction of flow of the first gas.
68. The breathing circuit according to any one of the preceding statements, wherein the breathing circuit further comprises a humidification device to humidify at least one of the first gas or the second gas.
69. The breathing circuit according to any one of the preceding statements, wherein the humidification device is configurated to humidify the first gas in the first passageway.
70. The breathing circuit according to preceding statement 68 or 69, wherein the humidification device is located on the first passageway.
71. The breathing circuit according to any one of statements 68 to 70, wherein the humidification device is configured to humidify the second gas.
72. The breathing circuit according to statement 71, wherein the humidification device humidifies only the first gas in the second passageway, in which the second gas is delivered to the second passageway downstream of the humidification device.
73. The breathing circuit according to any one of statements 68 to 71, wherein the humidification device is configured to humidify the first and second gases in the second passageway.
74. The breathing circuit according to any one of statements 71 to 73, wherein the humidification device receives and stores at least part of the second gas during patient exhalation.
75. The breathing circuit according to any one statements 68 to 74, wherein the humidification device humidifies both the first and second gases, in which the humidification device has dual chambers, one for each passageway, thereby allowing the first gas and the second gas to be humidified to different extents as required.
76. The breathing circuit according to any one of statements 68 to 75, wherein the breathing circuit has a splitter that splits flow of the first gas between the first and the second passageways, and the humidification device may be located upstream of the splitter.
77. The breathing circuit according to any one of the preceding statements, wherein the breathing circuit has a second gas inlet in the second passageway.
78. The breathing circuit according to statement 77 when appended to any one of the preceding statements 17 to 31, wherein the second gas inlet is downstream of the at least one valve or the active valve mechanism.
79. The breathing circuit according to statement 77 when appended to any one of the preceding statements 17 to 31, wherein the second gas inlet in the second passageway upstream of the active valve mechanism.
80. The breathing circuit according to any one of statements 77 to 79, wherein the second gas inlet is located in a distal portion of second passageway, and the second gas can flow toward the patient interface during patient exhalation and be stored therein.
81. The breathing circuit according to any one of clams 77 to 79, wherein the second gas inlet is located in a proximal portion of the second passageway, and the second gas can be conveyed in the second passageway in a direction away from the patient interface during patient exhalation and be stored in the second passageway.
82. The breathing circuit according to any one of statements 77 to 81, wherein the second gas inlet is located on an element that forms part of the second passageway.
83. The breathing circuit according to any one of statements 77 to 82, wherein the second gas inlet is on the humidification device that is in the second passageway.
84. The breathing circuit according to statement 83, wherein the second gas inlet supplies the second gas into the humidification device that is in the second passageway.
85. The breathing circuit according to any one of statements 77 to 82, wherein the second gas inlet is on a reservoir that is in the second passageway.
86. The breathing circuit according to statement 85, wherein the second gas inlet supplies the second gas into the reservoir that is in the second passageway.
87. The breathing circuit according to any one of clams 77 to 86 when appended to statement 68, wherein the humidification device is located upstream of the second gas inlet.
88. The breathing circuit according to any one of clams 77 to 86 when appended to statement 68, wherein the humidification device is located downstream of the second gas inlet.
89. The breathing circuit according to any one of the preceding clams, wherein the second passageway is configured to store the second gas during patient exhalation whilst the first gas is being supplied to the patient interface via the first passageway, so that the second gas accumulates in the second passageway independently of the supply of the breathing gas to the patient.
90. The breathing circuit according to statement 89, wherein the second passageway receives and stores the second gas during patient exhalation.
91. The breathing circuit according to statement 89 when appended to any one of the statement 68 to 76, wherein the second passageway and the humidification device receive and store the second gas during the patient exhalation.
92. The breathing circuit according to any one of the preceding statements, wherein the second passageway has an internal volume that is sized to receive and a store a desired amount of the second gas for the patient during patient exhalation, the desired amount of the second gas being an amount that can be inhaled by the patient in a single breath.
93. The breathing circuit according to statement 92, wherein the internal volume of the gas second passageway for storing the second gas has a length ranging from about 0.5 m to 2.5 m, or about a length ranging from 0.75 to 2.0 m, or a length ranging from about 1.5 to 1.8 m.
94. The breathing circuit according to statement 91 or 92, wherein the internal volume of the second passageway for storing the second gas has a constant diameter, in which the diameter ranges from about 8 to 15 mm, or the diameter is about 10 mm.
95. The breathing circuit according to any one of the preceding statements, wherein the breathing circuit comprises a flow controller that is operated to control the rate at which the second gas is delivered to the second passageway.
96. The breathing circuit according to any one of the preceding statements, wherein the breathing circuit delivers high flow therapy the first and second gases can be delivered to a (adult) patient at a flow rate greater than or equal to about 10 L/min, such as between about 10 L/min and about 100 L/min, or between about 15 L/min and about 95 L/min, or between about 20 L/min and about 90 L/min, or between about 25 L/min and about 85 L/min, or between about 30 L/min and about 80 L/min, or between about 35 L/min and about 75 L/min, or between about 40 L/min and about 70 L/min, or between about 45 L/min and about 65 L/min, or between about 50 L/min and about 60 L/min.
97. The breathing circuit according to any one of the preceding statements, wherein the breathing circuit delivers high flow therapy the first and second gases can be delivered to a (neonatal, infant, or child) patient at a flow rate of greater than 1 L/min, such as between about 1 L/min and about 25 L/min, or between about 2 L/min and about 25 L/min, or between about 2 L/min and about 5 L/min, or between about 5 L/min and about 25 L/min, or between about 5 L/min and about 10 L/min, or between about 10 L/min and about 25 L/min, or between about 10 L/min and about 20 L/min, or between about 10 L/min and 15 L/min, or between about 20 L/min and 25 L/min.
98. The breathing circuit according to to any one of statement 1 to 95, wherein the breathing circuit delivers CPAP therapy in which the first gas and second gases are delivered to the patient interface at a pressure greater than 2 cmH₂O, such as between about 2 and about 40 cmH₂O, and suitably between about 4 and about 30 cmH₂O.
99. The breathing circuit according to any one of the preceding statements, wherein the breathing circuit comprises a flow assembly that causes the first gas to be conveyed in the first passageway during patient inhalation, and cause the first gas and the second gas to be conveyed in the second passageway during patient exhalation.
100. The breathing circuit according to any one of the preceding statements, wherein the flow assembly that is connectable to a first passageway to supply a first gas during patient exhalation, and is connectable to a second passageway to supply the first gas and the second gas during patient inhalation.
101. The breathing circuit according to any one of the preceding statements, wherein the first gas is air.
102. A breathing circuit for providing respiratory support to a patient, the breathing circuit comprising:
   first and second passageways that can convey a breathing gas to a patient interface, wherein the first passageway is connectable to a flow assembly to supply a first gas, and the second passageway is connectable to the flow assembly to supply the first gas and a second gas,
   wherein the flow assembly and the first passageway are configured to supply the first gas to the patient interface during patient exhalation, and the flow assembly and the second passageway are configured to supply the first gas and the second gas to the patient interface during patient inhalation.
103. The breathing circuit according to statement 102, wherein the flow assembly causes the first gas to be conveyed in the first passageway during patient inhalation, and cause the first gas and the second gas to be conveyed in the second passageway during patient exhalation.
104. The breathing circuit according to statement 103, wherein the flow assembly that is connectable to a first passageway to supply a first gas during patient exhalation, and is connectable to a second passageway to supply the first gas and the second gas during patient inhalation.
105. The breathing circuit according to statement 104, wherein the first passageway is connectable to a first gas source to supply the first gas, and the second passageway is connectable to the first gas source and to a second gas source.
106. The breathing circuit according to any one of statements 102 to 105, wherein the flow assembly allows supply of the breathing gas to the patient interface to be maintained via one of the first and second passageways at all times, and allows supply of the second gas to the patient interface to be independent of supply of the first gas.
107. The breathing circuit according to any one of statements 102 to 106, wherein the flow assembly comprises the first gas source that supplies the first gas.
108. The breathing circuit according to statement 107, wherein the first gas source comprises a flow generator that generates a flow of the first gas.
109. The breathing circuit according to any one of statements 101 to 108, wherein the first gas is air.
110. The breathing circuit according to any one of statements 101 to 109, wherein the flow assembly comprises the second gas source that supplies the second gas, the second gas source being a pressurized gas source.
111. The breathing circuit according to statement 110, wherein the flow assembly comprises the second gas source that supplies the second gas, the second gas source being a pressurized gas source.
112. The breathing circuit according to statement 111, wherein the pressurized gas includes one or any combination of: oxygen gas, heliox, or an anaesthetic gas.
113. The breathing circuit according to statement 111 or 112, wherein the flow assembly supplies the second gas to the second passageway during patient inhalation.
114. The breathing circuit according to any one of statements 111 to 113, wherein the flow assembly supplies the second gas to the second passageway during patient exhalation.
115. The breathing circuit according to statement 111 or 112, wherein the flow assembly supplies the second gas to the second passageway during patient inhalation and during patient exhalation.
116. The breathing circuit according to any one of statements 101 to 115, wherein the flow assembly is operable to adjust the flow of the first gas in the first and the second passageways based on the breathing cycle of the patient, that is, based on patient inhalation and patient exhalation.
117. The breathing circuit according to any one of statements 101 to 116, wherein the flow assembly is operable to adjust a parameter of the first gas in the first passageway, in which the first gas source is operable to adjust the flow rate of the first gas supplied to either one or both of the first passageway and the second passageway to provide high flow therapy.
118. The breathing circuit according to any one of statements 101 to 116, wherein the flow assembly is operable to adjust a parameter of the first gas in the first passageway, in which the first gas source is operable to adjust the pressure of the first gas supplied to either one of, or both of, the first passageway and the second passageway to provide CPAP therapy or bi-level pressure therapy.
119. The breathing circuit according to any one of statements 101 to 116, wherein the flow assembly is operable to adjust a parameter of the first gas in the first passageway, in which the first gas source is operable to adjust the flow rate of the first gas supplied to either one or both of the first passageway and the second passageway to provide high flow therapy.
120. The breathing circuit according to any one of statements 107 to 119, wherein the first gas source is the flow generator and is connectable to the first and second passageways to divide flow from the flow generator to the first and the second passageways.
121. The breathing circuit according to any one of statements 107 to 120, wherein the flow assembly is operable to alternate flow of the first gas from the flow generator between the first passageway and the second passageway.
122. The breathing circuit according to any one of statements 107 to 121, wherein the flow assembly is operable to alternate flow of the first gas from the flow generator between the first passageway and the second passageway.
123. The breathing circuit according to statement 122, wherein the alternate flow of the first gas comprises there being:
   i) substantially no, or little flow, of the first gas in the first passageway during inhalation, and there being flow of the first gas in the second passageway during inhalation, and
   ii) substantially no or little flow of the first gas in the second passageway during exhalation, and there being flow of the first gas in the first passageway during exhalation.
124. The breathing circuit according to any one of statements 101 to 123, wherein the flow assembly comprises one or more valves to adjust the flow of the first gas in the first passageway and the second passageway.
125. The breathing circuit according to statement 124, wherein the one or more valves alternate the flow of the first gas between the first passageway and the second passageway.
126. The breathing circuit according to statement 124 or 125, wherein the one or more valves comprises two valves in which one valve is located in the first passageway, and a second is located on the second passageway.
127. The breathing circuit according to 124 or 125, wherein the one or more valves comprises a three-way valve having one inlet connectable to the first gas source and two outlets, in which one of the outlets is connectable to the first passageway and the second outlet is connectable to the second passageway.
128. The breathing circuit accord to any one of statements 124 to 127, wherein the one or more valves is an active valve mechanism.
129. The breathing circuit according to any one of statements 102 to 127, wherein the flow assembly comprises an active valve mechanism to adjust the flow of the first gas in the first passageway and the second passageway.
130. The breathing circuit according to any one of statements 102 to 127, wherein the flow assembly comprises an active valve mechanism to adjust the flow of the first gas in the first passageway and the second passageway.
131. The breathing circuit according to any one of statements 128 to 130, wherein the active valve mechanism is operable to alternate flow of the first gas between the first passageway and the second passageway so that the first gas can be delivered to the patient interface via the first or the second passageways at any one time.
132. The breathing circuit according to any one of statements 128 to 131, wherein the active valve mechanism comprises an actively-controlled valve that is operated by an actuator that receives an output signal from a sensor that sensors the breathing cycle of the patient.
133. The breathing circuit according to any one of statements 128 to 132, wherein the active valve mechanism is located in distal portions of the first and second passageways.
134. The breathing circuit according to any one of statements 128 to 132, wherein the active valve mechanism is located in proximal portions of the first and second passageways.
135. The breathing circuit according to any one of statements 128 to 134, wherein the active valve mechanism comprises an actively-controlled valve comprising a three-way valve to alternate flow of the first gas between the first passageway and the second passageway.
136. The breathing circuit according to statement 135, wherein the three-way valve is connected to a proximal portion of the first and second passageways and has a first port that is connectable to the first passageway, a second port that is connectable to the second passageway, and a third port that is connectable to the patient interface.
137. The breathing circuit according to statement 135 or 136, wherein the three-way valve is connected to a proximal portion of the first and second passageways and has a first port that is connectable to the first passageway, a second port that is connectable to the second passageway, and third port that is connectable to a first gas source.
138. The breathing circuit according to 128 to 136, wherein the active valve mechanism comprises first and second control valves in the first and second passageways to control flow of the first gas to the passageways.
139. The breathing circuit according to statement 138, wherein the first and second control valves are opened and closed alternately, that is, the first control valve opened and the second control valve closed during patient exhalation, and vice versa, the first control valve closed and the second control valve opened during patient inhalation.
140. The breathing circuit according to any one of statements 102 to 139, wherein the flow assembly includes a third passageway interconnecting the patient interface and a joiner that merges the first and the second passageways.
141. The breathing circuit according to statement 140, wherein the third passageway interconnects the patient interface and the joiner.
142. The breathing circuit according to any one of statements 108 to 116, wherein the flow generator is a single blower.
143. The breathing circuit according to any one of preceding statements when appended to statement 108, wherein the flow generator has a single flow output thereof is split between the first and the second passageways.
144. The breathing circuit according to any one of preceding statements when appended to statement 108, wherein the flow generator has an output that is split between the first passageway and the second passageways.
145. The breathing circuit according to any one of preceding statements when appended to statement 108, wherein the flow generator has two flow outputs, and each flow output can be delivered to either one of the respective first and second passageways.
146. The breathing circuit according to any one of preceding statements when appended to statement 108, wherein each flow output is driven by a separate blower.
147. The breathing circuit according to any one of preceding statements when appended to statement 108, wherein the flow generator comprises a first flow generator that conveys the first gas along the first passageway, and a two second flow generator that conveys the first gas along the second passageway.
148. The breathing circuit according to any one of statements 102 to 147 when appended to statement 108, wherein the breathing circuit further comprises a sensor for detecting the breathing cycle of the patient, that is, when patient inhalation and patient exhalation is occurring, and the sensor has an output signal that is used to operate the active valve mechanism.
149. The breathing circuit according to statement 148, wherein the sensor has an output signal that is used to operate the flow generator.
150. The breathing circuit according to statement 149, wherein the output signal can be used to adjust the flow rate of the first gas supplied by the flow generator for high flow therapy.
151. The breathing circuit according to statement 148 or 149, wherein the output signal can be used to adjust the pressure of the first gas supplied by the flow generator for CPAP or bi-level pressure therapy.
152. The breathing circuit according to statement 151, wherein the output signal can be used to adjust the pressure of the first gas supplied by the flow generator between a first pressure being an IPAP (inspiratory positive airway pressure) and a second pressure being an EPAP (expiratory positive airway pressure).
153. The breathing circuit according to any one of statements 148 to 152, wherein the sensor comprises at least one gas sensor that can detect at least one of: i) a gas property of the breathing gas supplied to the patient interface, ii) a property of exhaled gases, and/or iii) a property of gases being vented from the breathing circuit.
154. The breathing circuit according to any one of statements 148 to 153, wherein the sensor comprises multiple sensors located at a selection of the following locations: i) the patient interface; ii) an outlet of the flow generator, iii) in the first passageway, and iv) in the second passageway.
155. The breathing circuit according to any one of statements 153 or 154 when appended to statements 152, wherein the gas property comprises at least one of: gas flow rate, gas pressure, gas temperature, gas humidity or gas concentration, such as oxygen or carbon dioxide concentration.
156. The breathing circuit according to any one of statements 148 to 155, wherein the sensor includes a pressure sensor, located at or near an outlet of the flow generator, or located on the patient interface.
157. The breathing circuit according to any one of statements 148 to 156, wherein the sensor includes a pressure sensor at the second gas inlet, or at a vent in the first passageway, or at an exhalation portion.
158. The breathing circuit according to any one of statements 148 to 157, wherein the sensor includes a flow sensor, located at or near an outlet of the flow generator.
159. The breathing circuit according to any one of statements 148 to 158, wherein the sensor includes a flow sensor located at one or more of the following: i) at or near the patient interface; ii) at the second gas inlet.
160. The breathing circuit according to any one of statements 148 to 159, wherein the sensor includes a flow sensor located in one or more the following: i) in the first passageway, ii) in the second passageway, or iii) an exhalation vent.
161. The breathing circuit according to any one of statements 148 to 160, wherein an output signal of the sensors is used to determine if the patient is inhaling or exhaling and, in turn, control the active valve mechanism so that the first gas can be delivered to the first passageway during exhalation and the first gas delivered to the second passageway during inhalation at a desired flow rate or a desired pressure.
162. The breathing circuit according to any one of statements 148 to 161, wherein the sensor comprises an external sensor that detects a respiratory parameter of the patient.
163. The breathing circuit according to statement any one of statements 148 to 162, wherein the breathing circuit further comprises a controller that receives the output signal of the sensor(s), and the controller has a processor that calculates the period of inhalation and/or exhalation, and in turn, produce a control output that is used to operate the active valve mechanism.
164. The breathing circuit according to statement 163, wherein the controller calculates the period of inhalation, and generates an output signal that used to operate the active valve mechanism that minimizes the delay between the active valve mechanism delivering the first gas to the second passageway and the patient receiving the breathing gas from the second passageway at the start of patient inhalation.
165. The breathing circuit according to statement 162 or 164 when appended to any one of statements 108 or 120 to 121, wherein the flow generator is adjusted to control the flow rate of the first gas supplied to the first and second passageways based on the output signal of the controller for high flow therapy.
166. The breathing circuit according to statement 163 or 164 when appended to any one of statements 108 or 120 to 121, wherein the flow generator is adjusted to control the pressure of the first gas supplied to the first and second passageways based on the output signal of the controller for CPAP or bi-level therapy.
167. The breathing circuit according to any one of statements 102 to 166, wherein the breathing circuit comprises the patient interface which is an unsealed patient interface for high flow therapy.
168. The breathing circuit according to any one of statements 102 to 167, wherein the flow assembly comprises an outlet on the second passageway for venting part or all of a residual breathing gas from the second passageway during patient exhalation.
169. The breathing circuit according to any one of statements 102 to 167, wherein the flow assembly comprises an vent on the second passageway for venting part or all of a residual breathing gas from the second passageway during patient exhalation.
170. The breathing circuit according to any one of statements 102 to 169, wherein the vent is located downstream of the where the second gas enters the second passageway, so that the second gas entering the second passageway during patient exhalation displaces the residual gas though the outlet, in which the breathing gas displaced through the outlet comprises any of the first and/or the second gas in the second passageway not inhaled.
171. The breathing circuit according to statement 102 to 170, wherein the vent comprises any one or a combination of the control valve, PEEP (positive end-expiratory pressure) valve, an aperture of fixed size, or a controlled outlet that maintains a substantially constant venting flow across a range of pressures.
172. The breathing circuit according to any one of statements 102 to 166, wherein the breathing circuit comprises the patient interface which is a sealed patient interface for CPAP therapy or bi-level pressure therapy.
173. The breathing circuit according to statement 172, wherein the breathing circuit comprises an exhalation port for venting exhaled gas.
174. The breathing circuit according to statement 172, wherein the breathing circuit comprises an exhalation port for venting first gas from the first passageway to prevent over-pressurising the patient interface.
175. The breathing circuit according to statement 172, wherein the flow assembly comprises an exhalation port for venting exhaled gas from the breathing circuit.
176. The breathing circuit according to statement 172, wherein the flow assembly comprises an exhalation port for venting first gas from the first passageway to prevent over-pressurising the patient interface.
177. The breathing circuit according to statement 172, wherein the exhalation port is located on the patient interface.
178. The breathing circuit according to any one of statements 172 to 176, wherein the exhalation port is located on a proximal portion of the first passageway, which reduces the likelihood of the second gas being vented through the exhalation port without being inhaled by the patient.
179. The breathing circuit according to any one of statements 172 to 176, wherein the exhalation port is located on a distal portion of the first passageway, which reduces the likelihood of the second gas inadvertently being leaked from the circuit.
180. The breathing circuit according to any one of statements 102 to 179, wherein the second passageway includes a non-return valve to inhibit any residual amount of the breathing gas not inhaled with each breath from flowing upstream, that is, in a direction opposite to the direction of flow of the first gas.
181. The breathing circuit according to any one of statements 102 to 180, wherein the breathing circuit further comprises a humidification device to humidify the first gas in the first passageway.
182. The breathing circuit according to statement 181, wherein the humidification device is located on the first passageway.
183. The breathing circuit according to any one of statements 102 to 182, wherein the breathing circuit further comprises a humidification device to humidify the second gas in the second passageway.
184. The breathing circuit according to statement 183, wherein the humidification device is located on the second passageway.
185. The breathing circuit according to statement 181 or 182, wherein the humidification device humidifies only the first gas in the second passageway, in which the second gas is delivered to the second passageway downstream of the humidification device.
186. The breathing circuit according to statement 184, wherein the humidification device is configured to humidify the first and second gases in the second passageway.
187. The breathing circuit according to any one of statements 184 to 186, wherein the humidification device receives and stores at least part of the second gas during patient exhalation.
188. The breathing circuit according to statement 186, wherein the humidification device has dual chambers, one for each passageway, thereby allowing the first gas and the second gas to be humidified to different extents as required.
189. The breathing circuit according to any one of statements 181 to 188, wherein the flow assembly has a splitter that splits flow of the first gas between the first and the second passageways, and the humidification device may be located upstream of the splitter.
190. The breathing circuit according to any one of statement 102 to 189, wherein the breathing circuit comprises a second gas inlet in the second passageway.
191. The breathing circuit according to statement 190 when appended to any one of statements 128 to 139, wherein the breathing circuit has a second gas inlet in the second passageway upstream of the active valve assembly.
192. The breathing circuit according to statement 190 when appended any one of statements 128 to 139, wherein the breathing circuit has a second gas inlet in the second passageway downstream of the active valve assembly.
193. The breathing circuit according to statement 190 when appended any one of statements 128 to 139, wherein the flow assembly has a second gas inlet in the second passageway downstream of the active valve assembly.
194. The breathing circuit according to statement 190 when appended to any one of statements 128 to 139, wherein the flow assembly has a second gas inlet in the second passageway upstream of the active valve assembly.
195. The breathing circuit according to any one of statements 190, 191 or 194, wherein the second gas inlet is located in a distal portion of second passageway, and the second gas can flow toward the patient interface during patient exhalation and be stored therein.
196. The breathing circuit according to any one of statements 190, 192 or 193, wherein the second gas inlet is located in a proximal portion of the second passageway, and the second gas can be conveyed in the second passageway in a direction away from the patient interface during patient exhalation and be stored in the second passageway.
197. The breathing circuit according to any one of statements 190 to 196, wherein the second gas inlet is located on an element that forms part of the second passageway.
198. The breathing circuit according to any one of statements 190 to 197 when appended to any one of statements 181 to 189, wherein the second gas inlet is on the humidification device that is in the second passageway.
199. The breathing circuit according to any one of statements 190 to 197 when appended to any one of statements 181 to 189, wherein the second gas inlet supplies the second gas into the humidification device that is in the second passageway.
200. The breathing circuit according to any one of statements 190 to 199, wherein the second gas inlet is on a reservoir that is in the second passageway.
201. The breathing circuit according to statement 200, wherein the second gas inlet supplies the second gas into the reservoir that is in the second passageway.
202. The breathing circuit according to any one of statements 102 to 201, wherein the second passageway is configured to store the second gas during patient exhalation whilst the first gas is being supplied to the patient interface via the first passageway, so that the second gas accumulates in the second passageway independently of the supply of the breathing gas to the patient.
203. The breathing circuit according to any one of statements 102 to 202, wherein the second passageway receives and stores a volume of the second gas during patient exhalation.
204. The breathing circuit according to any one of statements 102 to 203, wherein the second gas is stored during the patient exhalation.
205. The breathing circuit according to any one of statements 181 to 189, wherein the second passageway and the humidification device receives and stores a volume of the second gas during patient exhalation.
206. The breathing circuit according to any one of statements 102 to 205, wherein the second passageway has an internal volume that is sized to receive and a store a desired amount of the second gas for the patient during patient exhalation, the desired amount of the second gas being an amount that can be inhaled by the patient in a single breath.
207. The breathing circuit according to statement 206, wherein the internal volume of the gas second passageway for storing the second gas has a length ranging from about 0.5 m to 2.5 m, or about a length ranging from 0.75 to 2.0 m, or a length ranging from about 1.5 to 1.8 m.
208. The breathing circuit according to statement 206 or 207, wherein the internal volume of the second passageway for storing the second gas has a constant diameter, in which the diameter ranges from about 8 to 15 mm, or the diameter is about 10 mm.
209. The breathing circuit according to any one of statements 102 to 208, wherein the flow assembly comprises a flow controller that is operated to control the rate at which the second gas is delivered to the second passageway.
210. The breathing circuit according to any one of statements 102 to 209, wherein the breathing circuit delivers high flow therapy the first and second gases can be delivered to a (adult) patient at a flow rate greater than or equal to about 10 L/min, such as between about 10 L/min and about 100 L/min, or between about 15 L/min and about 95 L/min, or between about 20 L/min and about 90 L/min, or between about 25 L/min and about 85 L/min, or between about 30 L/min and about 80 L/min, or between about 35 L/min and about 75 L/min, or between about 40 L/min and about 70 L/min, or between about 45 L/min and about 65 L/min, or between about 50 L/min and about 60 L/min.
211. The breathing circuit according to any one of statements 102 to 210, wherein the breathing circuit delivers high flow therapy the first and second gases can be delivered to a (neonatal, infant, or child) patient at a flow rate of greater than 1 L/min, such as between about 1 L/min and about 25 L/min, or between about 2 L/min and about 25 L/min, or between about 2 L/min and about 5 L/min, or between about 5 L/min and about 25 L/min, or between about 5 L/min and about 10 L/min, or between about 10 L/min and about 25 L/min, or between about 10 L/min and about 20 L/min, or between about 10 L/min and 15 L/min, or between about 20 L/min and 25 L/min.
212. The breathing circuit according to statement 102 to 209, wherein the breathing circuit delivers CPAP therapy in which the first gas and second gases are delivered to the patient interface at a pressure greater than 2 cmH₂O, such as between about 2 and about 40 cmH₂O, and suitably between about 4 and about 30 cmH₂O.
213. A method of providing respiratory support to a patient, the method comprising:
   supplying a first gas to a first passageway connected to a patient interface during patient exhalation;
   supplying a second gas to a second passageway connected to the patient interface; and
   wherein supplying the first gas to the second passageway displaces at least some of the second gas in the second passageway to the patient interface during patient inhalation.
214. The method according to statement 213, wherein the method further comprising storing the second gas in the second passageway during patient exhalation.
215. The method according to statement 213 or 214, wherein the method comprises maintaining supply of the breathing gas in at least one of the first passageway and the second passageway at all times, so that the supplying the second gas to the patient interface via the second passageway can be controlled independently of supply of the first gas to the patient interface.
216. The method according to any one of statement 213 to 215, wherein the method comprises supplying of the first gas to the patient interface via the first passageway, and supplying the first gas and the second gas to the patient interface via the second passageway based on the breathing cycle of the patient.
217. The method according to any one of statements 213 to 216, wherein the method comprises alternating supply of the first gas between the first passageway and the second passageway during patient exhalation and patent inhalation respectively.
218. The method according to any one of statements 213 to 217, wherein the method comprises operating one or more valves to control the supply of the first gas to the first passageway and to control the supply of the first gas to the second passageway.
219. The method according to any one of statements 213 to 217, wherein the method comprises operating an active valve mechanism to control the supply of the first gas to the first passageway and to control the supply of the first gas to the second passageway.
220. The method according to statement 219, wherein operating the active valve mechanism comprises operating an actively-controlled valve comprising a three-way valve to alternate flow of the first gas between the first passageway and the second passageway respectively.
221. The method according to statement 219, wherein operating the active valve mechanism comprises operating actively-controlled first and second control valves in the first and second passageways to control the supply of the first gas to the first and second passageways respectively.
222. The method according to statement 221, wherein the first and second control valves are opened and closed alternately, that is, the first control valve opened and the second control valve closed during patient exhalation, and vice versa, the first control valve closed and the second control valve opened during patient inhalation
223. The method according to any one of statements 213 to 222, wherein the method comprises sensing a respiration rate (or breathing cycle) of the patient; and operating an active valve mechanism to control flow of the first gas to the first passageway and the second passageway.
224. The method according to any one of statements 213 to 222, wherein the method comprises sensing at least one of a gas property of the breathing gas supplied to the patient interface, a property of exhaled gases, or a property of gases being vented from the breathing circuit.
225. The method according to statement 224, wherein the gas property includes any one or a combination of: gas flow rate, gas pressure, gas temperature, gas humidity or gas concentration, such as oxygen or carbon dioxide concentration.
226. The method according to any one of statements 213 to 225, wherein the method comprises calculating the period of inhalation and/or exhalation, and generating an output signal to operate the active valve mechanism.
227. The method according to statement 226, wherein the output signal generated to operate the active valve mechanism minimizes the delay between the active valve mechanism supplying the first gas to the second passageway and the patient receiving the breathing gas from the second passageway at the start of patient inhalation.
228. The method according to statement 227 when appended to any one of statements 219 to 222, wherein the output signal is used to operate the active valve mechanism comprises supplying the first gas to the second passageway before inhalation by a pre-emptive period.
229. The method according to any one of statements 213 to 228, wherein the method comprises operating a first gas source to control the flow rate and/or pressure of the first gas supplied to the first and the second passageways.
230. The method according to any one of statements 226 to 229, wherein the method comprises using the output signal to operate a first gas source to control the flow rate and/or pressure of the first gas supplied to the first and the second passageways.
231. The method according to statement 229, wherein operating the first gas source comprises operating a single flow generator to deliver the first gas.
232. The method according to statement 229 or 231, wherein operating the first gas source comprises operating first and second flow generators, in which the first flow generator provides a stream of the first gas to the first passageway and the second flow generator provides a stream of the first gas to the second passageway.
233. The method according to any one of statements 223 to 232, wherein the method comprises operating a second gas source to control the flow rate and/or pressure of the second gas into the second passageway.
234. The method according to statement 233, wherein the second gas is supplied to a distal portion of second passageway, and flows toward the patient interface during patient exhalation and is stored therein during patient exhalation.
235. The method according to statement 233, wherein the second gas is supplied to a proximal portion of the second passageway, and flows in a direction away from the patient interface during patient exhalation and is stored therein during patient exhalation.
236. The method according to any one of statements 213 to 235, wherein the method comprises providing high pressure therapy by controlling the flow rate of the first gas supplied to the first and second passageways and using an unsealed patient interface.
237. The method according to statement 236, wherein the method comprises venting part or all of a residual breathing gas from the second passageway during patient exhalation.
238. The method according to statement 237, wherein the method comprises venting the residual breathing gas via a vent that is located downstream of the where the second gas enters the second passageway, so that the second gas entering the second passageway during patient exhalation displaces the residual gas though the vent, in which the breathing gas displaced through the vent includes any of the first and/or the second gas in the second passageway not inhaled or vented during patient inhalation.
239. The method according to any one of statements 213 to 235, wherein the method comprises providing CPAP or bi-level therapy by controlling the pressure of the first gas supplied to the first and second passageways and using a sealed patient interface.
240. The method according to any one of statements 213 to 239, wherein the method comprises venting exhaled gas via an exhalation port, and, if required, venting first gas from the first passageway to prevent over-pressurising the patient interface.
241. The method according to statement 240, wherein the method comprises providing the exhalation port on the sealed patient interface.
242. The method according to statement 240, wherein the method comprises providing the exhalation port on a proximal portion of the first passageway, which reduces the likelihood of the second gas being vented through the exhalation port without being inhaled by the patient.
243. The method according to statement 240, wherein the method comprises providing the exhalation port on a distal portion of the first passageway, which further reduces the likelihood of the second gas inadvertently being leaked form the circuit.
244. The method according to any one of statements 213 to 243, wherein the method further comprises humidifying the first gas in the first passageway.
245. The method according to any one of statements 213 to 243, wherein the method comprises humidifying the first gas in the second passageway without humidifying the second gas in the second passageway.
246. The method according to any one of statements 213 to 244, wherein the method comprises humidifying the first and second gases in the second passageway.
247. The method according to statements any one of statements 213 to 246, wherein the method comprises humidifying both the first and second gases in separate humidification chambers, thereby allowing the first gas and the second gas to be humidified to different extents as required.
248. The method according to any one of statements 213 to 247, wherein the method comprises storing the second gas during patient exhalation whilst the first gas is being supplied to the patient interface via the first passageway, so that the second gas accumulates in the second passageway independently of the supply of the breathing gas to the patient.
249. The method according to any one of clams 213 to 248, wherein the second gas includes one or any combination of: oxygen gas, heliox, or an anaesthetic gas.
250. The method according to any one of statements 213 to 249, wherein the method further comprises controlling the rate at which the second gas is delivered to the second passageway.
251. The method according to any one of statements 213 to 250, wherein the method delivers high flow therapy in which the first and second gases are delivered to a (adult) patient interface at a flow rate greater than or equal to about 10 L/min, such as between about 10 L/min and about 100 L/min, or between about 15 L/min and about 95 L/min, or between about 20 L/min and about 90 L/min, or between about 25 L/min and about 85 L/min, or between about 30 L/min and about 80 L/min, or between about 35 L/min and about 75 L/min, or between about 40 L/min and about 70 L/min, or between about 45 L/min and about 65 L/min, or between about 50 L/min and about 60 L/min.
252. The method according to any one of statements 213 to 251, wherein the method delivers high flow therapy in which the first and second gases are supplied to a (neonatal, infant, or child) patient interface at a flow rate of greater than 1 L/min, such as between about 1 L/min and about 25 L/min, or between about 2 L/min and about 25 L/min, or between about 2 L/min and about 5 L/min, or between about 5 L/min and about 25 L/min, or between about 5 L/min and about 10 L/min, or between about 10 L/min and about 25 L/min, or between about 10 L/min and about 20 L/min, or between about 10 L/min and 15 L/min, or between about 20 L/min and 25 L/min.
253. The method according to any one of statements 213 to 250, wherein the method delivers CPAP therapy in which the first gas and second gases are delivered to the patient interface at a pressure greater than 2 cmH₂O, such as between about 2 and about 40 cmH₂O, and suitably between about 4 and about 30 cmH₂O.
254. The method according to any one of statements 213 to 253, wherein the first gas is air.
255. A breathing circuit for providing respiratory support to a patient, the breathing circuit including:
   first and second passageways that convey a breathing gas to a patient interface, and
   a flow assembly that is connectable to a first passageway to supply a first gas during patient exhalation, and is connectable to a second passageway to supply the first gas and a second gas during patient inhalation.
256. A breathing circuit for providing respiratory support to a patient, the breathing circuit including:
   first and second passageways that convey a breathing gas to a patient interface, wherein the first passageway is connectable to a first gas source to supply a first gas, and the second passageway is connectable to the first gas source and to a second gas source to supply the first gas and a second gas respectively, and
   a flow assembly that is operably connected to the first and second passageways to cause the first and second gases to flow along the second passageway during patient inhalation and to cause the first gas to flow along the first passageway during patient exhalation.
257. A breathing circuit for providing respiratory support to patient, the breathing circuit including: first and second passageways that convey a breathing gas to a patient interface, wherein the first passageway is connectable to a first gas source to supply a first gas, and the second passageway is connectable to the first gas source and to a second gas source to supply the first gas and a second gas respectively,
   wherein the first passageway is configured to convey the first gas during patient exhalation and the second passageway is configured to convey the first gas and the second gas during patient inhalation.
258. A breathing circuit for providing respiratory support to a patient, the breathing circuit including:
   first and second passageways that convey a breathing gas to a patient interface, wherein the first passageway is connectable to a flow assembly to supply a first gas, and the second passageway is connectable to the flow assembly to supply the first gas and a second gas, wherein the flow assembly and the first passageway are configured to supply the first gas to the patient interface during patient exhalation, and the flow assembly and the second passageway are configured to supply the first gas and the second gas to the patient interface during patient inhalation.
259. A method of providing respiratory support to a patient, the method including the steps of:
   providing a breathing circuit for providing respiratory support to a patient, the breathing circuit having first and second passageways that convey a breathing gas to a patient interface, and a flow assembly that is connectable to a first passageway and is connectable to a second passageway; and
   operating the flow assembly so that first passageway supplies the first gas to the patient interface during patient exhalation, and the second passageway supplies the first gas and the second gas to the patient interface during patient inhalation.

Reference Numeral Table

| | | | |
|---|---|---|---|
| breathing circuit | 20 | method of providing respiratory | 60 |
| | | support | |
| first passageway | 21 | providing a breathing circuit | 61 |
| second passageway | 22 | operating the flow assembly | 62 |
| third passageway | 23 | operating an active valve mechanism | 63 |
| flow assembly | 24 | sensing a gas flow parameter | 64 |
| first gas source | 25 | sensing a respiration rate | 65 |
| first gas | 26 | operating a second gas source | 66 |
| second gas source | 27 | operating a first gas source | 67 |
| second gas | 28 | humidifying the breathing gas | 68 |
| distal portion of the second | 29 | actuator | 69 |
| passageway | | | |
| proximal portion of the second | 30 | exhalation port | 70 |
| passageway | | | |
| distal portion of the first passageway | 31 | unsealed interface | 71 |
| proximal portion of the first | 32 | sealed interface | 72 |
| passageway | | | |
| flow generator | 33 | patient | 74 |
| first flow generator | 33A | vent sensor | 75 |
| second flow generator | 33B | control valve | 76 |
| first gas inlet | 34 | | |
| second gas inlet | 35 | | |
| active valve mechanism | 36 | | |
| first non-return valve | 37 | | |
| second non-return valve | 38 | | |
| control valve | 43 | | |
| patient interface | 44 | | |
| venting valve | 45 | | |
| humidification device(s) | 46 | | |
| reservoir | 47 | | |
| sensor(s) | 49 | | |
| first humidification device | 50 | | |
| second humidification device | 51 | | |
| controller | 52 | | |
| first valve | 55 | | |
| second valve | 56 | | |

1. A breathing circuit for providing respiratory support to patient, the breathing circuit comprising:
a first passageway and a second passageways that can convey a breathing gas to a patient interface, wherein the first passageway is connectable to a first gas source to supply a first gas, and the second passageway is connectable to the first gas source and to a second gas source, the second gas source to supply a second gas,
wherein the first passageway can convey the first gas during patient exhalation and the second passageway supply the first gas and the second gas during patient inhalation.

2. The breathing circuit according to claim 1, wherein the first passageway and the second passageway can convey the breathing gas to the patient interface at all times, and can convey the second gas to the patient interface independently of the first gas being conveyed to the patient interface by the first passageway.

3. (canceled)

4. (canceled)

5. The breathing circuit according to claim 1, wherein the breathing circuit comprises the second gas source that supplies the second gas, the second gas source being a pressurized gas source.

6. (canceled)

7. (canceled)

8. The breathing circuit according to claim 5, wherein the second gas source supplies the second gas to the second passageway during patient exhalation.

9. (canceled)

10. The breathing circuit according to claim 1, wherein the breathing circuit is operable to adjust a flow of the first gas in the first passageway and the second passageway based on a breathing cycle of the patient, that is, based on patient inhalation and patient exhalation.

11. The breathing circuit according to claim 1, wherein the breathing circuit is operable to alternate flow of the first gas between the first passageway and the second passageway.

12. (canceled)

13. (canceled)

14. (canceled)

15. (canceled)

16. The breathing circuit according to claim 11, wherein an alternate flow of the first gas comprises there being:
substantially no, or little flow, of the first gas in the first passageway during inhalation, and there being flow of the first gas in the second passageway during inhalation, and
substantially no or little flow of the first gas in the second passageway during exhalation, and there being flow of the first gas in the first passageway during exhalation.

17. (canceled)

18. (canceled)

19. (canceled)

20. (canceled)

21. (canceled)

22. The breathing circuit according to claim 1, wherein the breathing circuit comprises an active valve mechanism to adjust a flow of the first gas in the first passageway and the second passageway.

23. (canceled)

24. (canceled)

25. (canceled)

26. (canceled)

27. (canceled)

28. (canceled)

29. (canceled)

30. The breathing circuit according to claim 22, wherein the breathing circuit further comprises a sensor for detecting a breathing cycle of the patient, that is, when patient inhalation and patient exhalation is occurring, and the sensor has an output signal that is used to operate the active valve mechanism.

31. (canceled)

32. (canceled)

33. (canceled)

34. (canceled)

35. (canceled)

36. (canceled)

37. (canceled)

38. (canceled)

39. (canceled)

40. (canceled)

41. The breathing circuit according to claim 1, wherein the breathing circuit comprises the patient interface which is a sealed patient interface for CPAP therapy or bi-level pressure therapy.

42. The breathing circuit according to claim 1, wherein the breathing circuit comprises a vent on the second passageway for venting part or all of a residual breathing gas from the second passageway during patient exhalation.

43. The breathing circuit according to claim 42, wherein the vent is located downstream of where the second gas enters the second passageway, so that the second gas entering the second passageway during patient exhalation displaces residual gas though the vent, in which the breathing gas displaced through the vent comprises any of the first gas and/or the second gas in the second passageway not inhaled.

44. (canceled)

45. The breathing circuit according to claim 41, wherein the breathing circuit comprises an exhalation port for venting exhaled gas from the breathing circuit.

46. (canceled)

47. The breathing circuit according to claim 45, wherein the exhalation port is located on the patient interface.

48. The breathing circuit according to claim 45, wherein the exhalation port is located on a proximal portion of the first passageway, which reduces a likelihood of the second gas being vented through the exhalation port without being inhaled by the patient.

49. The breathing circuit according to claim 45, wherein the exhalation port is located on a distal portion of the first passageway, which reduces a likelihood of the second gas inadvertently being leaked from the breathing circuit.

50. The breathing circuit according to claim 45, wherein the second passageway includes a non-return valve to inhibit any residual amount of the breathing gas not inhaled with each breath from flowing upstream, that is, in a direction opposite to the direction of flow of the first gas.

51. (canceled)

52. (canceled)

53. (canceled)

54. (canceled)

55. The breathing circuit according to claim 1, wherein the breathing circuit has a second gas inlet in the second passageway.

56. (canceled)

57. (canceled)

58. The breathing circuit according to claim 55, wherein the second gas inlet is located in a distal portion of second passageway, and the second gas can flow toward the patient interface during patient exhalation and be stored therein.

59. The breathing circuit according to claim 55, wherein the second gas inlet is located in a proximal portion of the second passageway, and the second gas can be conveyed in the second passageway in a direction away from the patient interface during patient exhalation and be stored in the second passageway.

60-90. (canceled)
